(19) United States
(12) Patent Application Publication
(10) Pub. No.: US 2007/0142638 A1
(43) Pub. Date: Jun. 21, 2007

(54) Ornithine derivatives as prostaglandin e2 agonists or antagonists

(75) Inventors: Kouji Hattori, Tokyo (JP); Naoaki Fujii, Germantown, TN (US); Akira Tanaka, Tokyo (JP); Kenichi Washizuka, Tokyo (JP); Minoru Sakurai, Tokyo (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: Astellas Pharma Inc., Chuo-ku (JP)

(21) Appl. No.: 10/584,146

(22) PCT Filed: Dec. 17, 2004
(86) PCT No.: PCT/JP04/19454

§ 371 Date: Dec. 28, 2006

(30) Foreign Application Priority Data
Dec. 20, 2003 (AU) 2003907110

(51) Int. Cl. C07D 215/38; C07D 209/42

(52) U.S. Cl. 546/169; 548/492; 549/468

(57) ABSTRACT

Ornithine derivatives of the formula (I): wherein X is —CO— or —(CH₂)ₖ— (wherein k is 1, 2 or 3); Y is Z-(CH₂)ₙ—, and the like; {wherein Z is R¹—CO—NR⁴—, and the like, (wherein R¹ is aryl, and the like; and R⁴ is hydrogen, or lower alkyl); and n is 1, 2, 3, 4, 5 or 6}; R² is aryl-(lower alkyl), and the like; R³ is -Q-R⁷, [wherein Q is —CO— or —SO₂—, R is heterocyclyl], and the like; and R⁵ and R⁶ are independently hydrogen or lower alkyl; or a pharmaceutically acceptable salt thereof, which are useful as medicament.

### TECHNICAL FIELD

This invention relates to new ornithine derivatives and pharmaceutically acceptable salts thereof which are useful as prostaglandin E₂ (hereinafter described as PGE₂) agonist or antagonist.

### BACKGROUND ART

PGE₂ is known as one of the metabolites in an arachidonate cascade. It is also known that PGE₂ has various activities such as pain inducing activity, pro- or anti-inflammatory activity, uterine contractile activity, a promoting effect on digestive peristalsis, an awaking activity, a suppressive effect on gastric acid secretion, hypotensive activity, platelet inhibition activity, bone-resorbing activity, angiogenic activity, or the like.

PGE₂-sensitive receptors have been sub-divided into four subtypes, EP1, EP2, EP3 and EP4, and these receptors have a wide distribution in various tissues. The effects associated with EP1 receptor activator are believed to be mediated by mobilization of Ca²⁺ from intracellular stores. The EP3 receptor is an example of promiscuous receptor that may couple to different second-messenger systems. Further, the effects associated with EP2 and EP4 receptors activator may be considered as inhibitory, and are believed to be associated with a stimulation of adenylate cyclase and an increase in levels of intracellular cyclic AMP. Especially, EP4 receptor may be considered to be associated with smooth muscle relaxation, anti-inflammatory or pro-inflammatory activities, lymphocyte differentiation, antiallergic activities, kidney dysfunction, mesangial cell relaxation or proliferation, gastric or enteric mucus secretion, or the like.

PGE₂ receptor blockers, in other words “PGE₂ antagonists”, possess binding activities to PGE₂-sensitive receptors. Accordingly, they possess a PGE₂-antagonizing or PGE₂-inhibiting activity. Therefore, they are expected as a medicament to treat and prevent PGE₂ mediated diseases. Similarly, PGE₂ agonists can be medicaments for PGE₂ mediated diseases. These PGE₂ agonists or antagonists are expected as a medicament to treat and prevent EP4 receptors-mediated diseases, such as kidney dysfunction, inflammatory conditions, various pains, or the like in human beings or animals.

Such PGE₂ antagonist is known. For example, in WO 00/16760 and WO 00/18744, oxazole compounds are disclosed.

### DISCLOSURE OF INVENTION

Under the above situation, the inventors of the present invention found that the compounds having an ornithine skeleton or ornithine derivative skeleton bind preferentially to PGE₂ receptor, therefore they can be good PGE₂ agonists or antagonists, particularly EP4 receptor blockers. As the result, the inventors completed this invention.

Accordingly, the present invention relates to novel ornithine derivatives which are useful for treating or preventing PGE₂ mediated diseases. One object of this invention is to provide new compound and pharmaceutically acceptable salt thereof as prostaglandin E₂ agonists or antagonists.

Another object of this invention is to provide a medicament and pharmaceutical composition containing the compound as an active ingredient.

A further object of this invention is to provide an agonist or antagonist of PGE₂ consisting of the ornithine derivative and a method for treatment and/or prevention of PGE₂ mediated diseases which comprises administering an effective amount of the ornithine derivative.

A further object of the present invention is to provide a use of the ornithine derivative.

A further object of the present invention is to provide the compound and pharmaceutically acceptable salt thereof which are useful for the manufacture of medicaments for treating or preventing conditions mediated by PGE₂, more particularly useful for treating or preventing kidney dysfunction, inflammatory conditions, various pains, collagen diseases, autoimmune diseases, various immunity diseases, analgesic, thrombosis, allergic disease, cancer and neurodegenerative diseases.

A further object of this invention is to provide the commercial package comprising the pharmaceutical composition containing the ornithine derivative.

The ornithine derivative of this invention can be represented by the following formula (I): 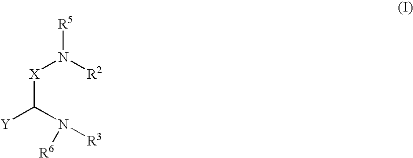
wherein

X is —CO— or —(CH₂)ₖ— (wherein k is 1, 2 or 3);

Y is
(1) lower alkyl, or
(2) Z-(CH₂ )ₙ—,
   {wherein
      Z is
         (1) aryl, or
         (2) R¹—CO—NR⁴—
         (wherein
         R¹ is (1) aryl, heterocyclyl, aryl-(lower alkyl), aryl-(lower alkoxy), or heterocyclyl-(lower alkoxy), each of which may be substituted with one or more substituent (s) selected from the group consisting of
         (a) lower alkyl,
         (b) halogen and
         (c) hydroxy; or
         (2) lower alkoxy; and
         R⁴ is hydrogen, or lower alkyl); and
      n is 1, 2, 3, 4, 5 or 6};
R² is (1) lower alkyl, aryl-(lower alkyl) or (lower alkyl)thio-(lower alkyl), each of which may be substituted with one or more substituent(s) selected from the group consisting of
   (a) heterocyclyl,
   (b) carboxy,
   (c) carboxy-(lower alkyl),
   (d) amidated carboxy,
   (e) (lower alkoxy)carbonyl which may be substituted with cycloalkyl, heterocyclyl or (lower alkanoyl)oxy; and
   (f) cyano; or
(2) aryl which may be substituted with lower alkyl, lower alkenyl, aryl, lower alkoxy, (lower alkyl)amino, (lower alkyl)thio, carboxy, (lower alkoxy)carbonyl, (lower alkoxy)-(lower alkyl), (lower alkyl)amino-(lower alkyl), or (lower alkyl)thio-(lower alkyl), each of which may be further substituted with one or more substituent (s) selected from the group consisting of
   (a) heterocyclyl,
   (b) (lower alkoxy)carbonyl,
   (c) carboxy and
   (d) amidated carboxy;
R³ is (1) -Q-R⁷,
   [wherein
   Q is —CO— or —SO₂—,
   R⁷ is (a) lower alkyl which may be substituted with

one or more substituent (s)selected from the group consisting of
cycloalkyl, aryl which may be further substituted with aryl(s), and heterocyclyl,
(b) lower alkenyl which may be substituted with one or more substituent(s) selected from the group consisting of aryl and heterocyclyl,
(c) cycloalkyl,
(d) aryl which may be substituted with one or more substituent (s) selected from the group consisting of
   lower alkyl, aryl which may be further substituted with hydroxy(s), lower alkoxy, aryloxy, hydroxy, and halogen,
(e) heterocyclyl which may be substituted with one or more substituent (s) selected from the group consisting of
   lower alkyl, aryl which may be further substituted with halogen(s), and halogen,
(f) aryloxy, or
(g) amino which may be substituted with aryl(s) which may be further substituted with one or more substituent(s) selected from the group consisting of aryl and heterocyclyl]; or
(2) lower alkyl which may be substituted with aryl(s) or heterocyclyl(s), each of which may be further substituted with aryl (s); and

R⁵ and R⁶ are independently hydrogen or lower alkyl; or

R⁶ and Y may be linked together to form —(CH₂)ₘ— (wherein m is 2, 3, 4 or 5);

or a pharmaceutically acceptable salt thereof.

In the above and subsequent description of the present specification, suitable examples of the various definitions to be included within the scope of the invention are explained in detail in the following.

The term “lower” is intended to mean a group having 1 to 6 carbon atom(s), unless otherwise provided.

Therefore, the “lower alkyl” means a straight or branched chain aliphatic hydrocarbon, such as methyl, ethyl, propyl, isopropyl, butyl, isobutyl, tert-butyl, pentyl, hexyl, and the like. It is preferably (C1-C4)alkyl, more preferably (C1-C2)alkyl, most preferably methyl.

The “lower alkenyl” means a straight or branched chain aliphatic hydrocarbon having more than one double bond between two carbon atoms, such as ethenyl, 1-methylethenyl, 1-propenyl, 2-propenyl, 1-methyl-l-propenyl, 2-butenyl, 3-butenyl, 3-methyl-2-butenyl, pentenyl, hexenyl, and the like, and it is preferably (C2-C5)alkenyl, more preferably (C2-C3)alkenyl, most preferably ethenyl.

The “cycloalkyl” means C3-C10 cycloalkyl group, such as cyclopropyl, cyclobutyl, cyclopentyl, cyclohexyl, cycloheptyl, norbornyl, adamantyl, and the like, and it is preferably (C5-C6)cycloalkyl.

The “aryl” means an aromatic hydrocarbon group, such as phenyl, naphthyl, indenyl, and the like, and it is preferably (C6-C10)aryl, more preferably naphthyl or phenyl, most preferably phenyl.

The “heterocyclyl” may include saturated or unsaturated, monocyclic or polycyclic heterocyclic group containing at least one hetero atom selected from nitrogen, sulfur and oxygen atom. The group preferably includes, for example:

saturated monocyclic heterocyclic group having 3 to 8-membere containing 1 to 4 nitrogen atom(s), such as pyrrolidinyl, imidazolidinyl, piperidinyl, piperazinyl, azacycloheptyl, azacyclooctyl, perhydroazepinyl, and the like;

monocyclic heteroaryl group containing 1 to 4 nitrogen atom(s), such as pyrrolyl, pyrrolinyl, imidazolyl, pyrazolyl, pyridyl and its N-oxide, pyrimidinyl, pyrazinyl, dihydropyridazinyl, tetrahydropyridazinyl, triazolyl (e.g., 1H-1,2,4-triazolyl, 1H-1,2,3-triazolyl, 2H-1,2,3-triazolyl, and the like), tetrazolyl (e.g., 1H-tetrazolyl, 2H-tetrazolyl, and the like), dihydrotriazinyl (e.g., 4,5-dihydro-1,2,4-triazinyl, 2,5-dihydro-1,2,4-triazinyl, and the like);

condensated heteroaryl group containing 1 to 5 nitrogen atom(s), such as indolyl, 2,3-dihydroindolyl, isoindolyl, indolyl, 1-methylindolyl, indazolyl, isoindolyl, indolizinyl, benzimidazolyl, quinolinyl, 1,2,3,4-tetrahydroquinolyl, isoquinolyl, benzotriazolyl, tetrazolopyridyl, imidazopyridinyl, methylimidazopyridinyl, tetrazolo-pyridazinyl (e.g., tetrazolo[1,5-b]pyridazinyl, and the like), dihydrotriazolopyridazinyl, quinoxalinyl;

monocyclic heteroaryl group having 3 to 8-membere containing 1 to 4 oxygen atom(s), such as furyl, pyranyl, and the like;

condensated heteroaryl group containing 1 to 4 oxygen atom(s), such as benzofuranyl, chromenyl, and the like;

monocyclic heteroaryl group having 3 to 8-membere containing 1 to 2 sulfur atom(s), such as thienyl, thiepinyl, and the like;

condensated heteroaryl group containing 1 to 5 sulfur atom(s), such as benzothienyl, naphto[2,3-b]thienyl, thianthrenyl, benzothienyl, benzothieteyl;

saturated monocyclic heterocyclic group having 3 to 8-membere containing 1 to 3 nitrogen atom(s) and 1 to 2 oxygen atom(s), such asmorpholino, and the like;

monocyclic heteroaryl group having 3 to 8-membere containing 1 to 3 nitrogen atom(s) and 1 to 2 oxygen atom(s), such as oxazolyl, isoxazolyl, dihydroisoxazolyl, oxadiazolyl (e.g., 1,2,4-oxadiazolyl, 1,3,4-oxadiazolyl, 2,5-oxadiazolyl, and the like);

condensated heteroaryl group containing 1 to 3 nitrogen atom(s) and 1 to 2 oxygen atom(s), such as berizoxazolyl, benzoxadiazolyl, and the like;

saturated monocyclic heterocyclic group having 3 to 8-membere containing 1 to 3 nitrogen atom(s) and 1 to 2 sulfur atom(s), such as thiazolidinyl;

monocyclic heteroaryl group having 3 to 8-membere containing 1 to 3 nitrogen atom(s) and 1 to 2 sulfur atom(s), such as thiazolyl, isothiazolyl, thiazolinyl, thiadiazolyl (e.g., 1,2,4-thiadiazolyl, 1,3,4-thiadiazolyl, 1,2,5-thiadiazolyl, 1,2,3-thiadiazolyl);

condensated monocyclic heteroaryl group containing 1 to 3 nitrogen atom(s) and 1 to 2 sulfur atom(s), such as benzothiazolyl, benzothiadiazolyl, and the like.

The “(lower)alkoxy” means a straight or branched chain aliphatic hydrocarbon oxy group, such as methoxy, ethoxy, propoxy, isopropoxy, butoxy, isobutoxy, tert-butoxy, pentoxy, hexoxy, and the like. It is preferably (C1-C4)alkoxy, more preferably (C1-C2)alkoxy.

The “(lower alkyl)amino” means a amino group substituted by the above lower alkyl group, such as methylamino, ethylamino, propylamino, isopropylamino, butylamino, isobutylamino, tert-butylamino, pentylamino, hexylamino, and the like. It is preferably [(C1-C4)alkyl]amino, more preferably [(C1-C2)alkyl]amino.

The “(lower alkyl)thio” means a sulfur atom (II) substituted by the above lower alkyl group, such as methylthio, ethylthio, propylthio, isopropylthio, butylthio, isobutylthio, tert-butylthio, pentylthio, hexylthio, and the like. It is preferably [(C1-C4)alkyl]thio, more preferably [(C1-C2)alkyl]thio.

The “aryloxy” means oxy group substituted with the above aryl, and includes phenyloxy, naphthyloxy, indenyloxy, and the like, and it is preferably phenyloxy.

The “halogen” may include a fluorine atom, a chlorine atom, a bromine atom and an iodine atom, more preferably a fluorine atom or a chlorine atom, most preferably a chlorine atom.

The “amidated carboxy” may include carbamoyl which may be substituted with aryl-(lower alkyl), e.g., benzyl, phenylethyl, phenylpropyl, or the like.

The “(lower alkoxy)carbonyl” means a carbonyl group substituted with lower alkoxy group mentioned above, such as methoxycarbonyl, ethoxycarbonyl, isopropoxycarbonyl, tert-butoxycarbonyl, and the like, and it is preferably [(C1-C4) alkoxy]carbonyl.

The “(lower alkanoyl)oxy” means a formyloxy and a (lower alkyl) carbonyloxy group such as acetyloxy, propionyloxy, butyryloxy, tert-butyryloxy, isobutyryloxy, valeryloxy, isovaleryloxy, pivaloyloxy, hexanoyloxy, and the like. It is preferably [(C1-C4)alkanoyl]oxy (including formyloxy).

The “aryl-(lower alkyl)”, “(lower alkoxy)-(lower alkyl)”, “(lower alkyl)amino-(lower alkyl)”, “(lower alkyl)thio-(lower alkyl)” and “carboxy-(lower alkyl)” mean the above lower alkyl group substituted with the above aryl, lower alkoxy, (lower alkyl)amino, (lower alkyl)thio and carboxy, respectively.

The “aryl-(lower alkoxy)” and “heterocyclyl-(lower alkoxy)” mean the above lower alkyl group substituted with the above aryl and heterocyclyl, respectively. For example, “aryl-(lower alkoxy)” may include benzyloxy, 1-phenylethoxy, 2-phenylethoxy, 3-phenylpropoxy, 4-phenylbutoxy, naphthylmethoxy, 2-naphthylethoxy, and the like. It is preferably phenyl-(lower alkoxy), more preferably phenyl[(C1-C4)alkoxy], more preferably phenyl[(C1-C2 )alkoxy], most preferably benzyloxy.

In case where the above groups are substituted, the number of substituent maybe two or more if feasible. When the number of substituent is plural, they may be identical or different to each other. In addition, the substituted position is not also limited. For example, when “aryl-(lower alkyl)” is substituted, the substituted position may be aryl moiety or lower alkyl moiety.

The Compound (I) contains one or more asymmetric centers and thus they can exist as enantiomers or diastereoisomers. The present invention includes both mixtures and separate individual isomers. However, at the carbon bonded by X, Y and N in Compound (I), (S) isomer is more preferable.

The compounds of the formula (I) may also exist in tautomeric forms and this invention includes both mixtures and separate individual tautomers.

The Compound (I) and their salt may be in a form of a solvate such as hydrate. Such a solvate is included within the scope of the present invention.

Also radiolabelled derivatives of Compound (I) which is suitable for biological studies are included in the scope of the present invention.

In the scope of the present invention, the prodrug of the Compound (I) is included, such a prodrug is capable of undergoing metabolic conversion to Compound (I) following administration in body. Further, in the scope of the present invention, metabolites of Compound (I) is included, which metabolites are therapeutically active in the treatment of the targeted medical condition.

The compound of the present invention can be converted to salt according to a conventional method. Suitable salt of the compounds (I) is pharmaceutically acceptable conventional non-toxic salts and include anorganic acid salt (e.g., acetate, maleate, tartrate, methanesulfonate, benzenesulfonate, formate, toluenesulfonate, trifluoroacetate, or the like), an inorganic acid salt (e.g., hydrochloride, hydrobromide, sulfate, phosphate, or the like), a salt with an amino acid (e.g., aspartate, glutamate, or the like), or the like.

Preferred embodiments of the Compound (I) is Compound (Ia) as follows: 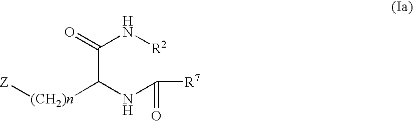
wherein R², R⁷, n and Z are as defined above.

More preferred embodiments of the Compound (I) is Compound (Ib) as follows: 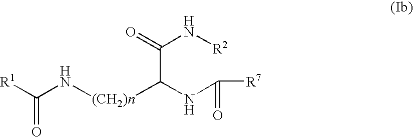
wherein R¹, R², R⁷ and n are as defined above.

As Compound (Ib), the compound having the following definition is more preferable:
R¹ is aryl-(lower alkoxy);
R² is lower alky, or aryl which may be substituted with carboxy-(lower alkyl);
R⁷ is heterocyclyl which may be substituted with substituted with lower alkyl; and
n is 1, 2, 3, 4 or 5.

In the each definition of the Compound (I), preferably,
(1) x is —CO—;
(2) X is or —(CH₂)ₖ— (wherein k is 1 , 2 or 3);
(3) Y is lower alkyl;
(4) Y is Z-(CH₂)ₙ—, wherein Z is aryl, n is 1, 2, 3, 4, 5 or 6;
(5) Y is Z-(CH₂)ₙ—, wherein Z is R¹—CO—NR⁴—; wherein R¹ is aryl or heterocyclyl, each of which may be substituted with one or more substituent(s) selected from the group consisting of lower alkyl, halogen and hydroxy; R⁴ is hydrogen; and n is 1, 2, 3, 4, 5 or 6;
(6) Y is Z-(CH₂)ₙ—, wherein Z is R¹—CO—NR⁴—; wherein R¹ is aryl-(lower alkyl) which may be substituted with one or more substituent (s) selected from the group consisting of lower alkyl, halogen and hydroxy; R⁴ is hydrogen; and n is 1, 2, 3, 4, 5 or 6;
(7) Y is Z-(CH₂)ₙ—, wherein Z is R¹—CO—NR⁴—; wherein R¹ is aryl-(lower alkoxy) or heterocyclyl-(lower alkoxy), each of which may be substituted with one or more substituent(s) selected from the group consisting of lower alkyl, halogen and hydroxy; R⁴ is hydrogen; and n is 1, 2, 3, 4, 5 or 6;
(8) Y is Z-(CH₂)ₙ—, wherein Z is R¹—CO—NR⁴—; wherein R¹ is aryl-(lower alkoxy) which may be substituted with one or more substituent (s) selected from the group consisting of lower alkyl, halogen and hydroxy; R⁴ is hydrogen; and n is 1, 2, 3, 4, 5 or 6;
(9) Y is Z-(CH₂)ₙ—, wherein Z is R¹—CO—NR⁴—; wherein R¹is phenyl-(lower alkoxy) which maybe substituted with one or more substituent(s) selected from the group consisting of lower alkyl, halogen and hydroxy; R⁴ is hydrogen; and n is 4, 5 or 6;
(10 ) Y is Z-(CH₂)ₙ—, wherein Z is R¹—CO—NR⁴—; wherein R¹ is benzyl which may be substituted with one or more substituent(s) selected from the group consisting of lower alkyl, halogen and hydroxy; R⁴ is hydrogen; and n is 4, 5 or 6;
(11) R²is aryl-(lower alkyl) which may be substituted with one or more substituent (s) selected from the group consisting of heterocyclyl, carboxy, carboxy-(lower alkyl), amidated carboxy, (lower alkoxy) carbonyl which may be substituted with cycloalkyl, heterocyclyl or (lower alkanoyl)oxy; and cyano;
(12) R² is aryl which may be substituted with lower alkyl, lower alkenyl, aryl, lower alkoxy, (lower alkyl)amino, (lower alkyl)thio, carboxy, (lower alkoxy)carbonyl, (lower alkoxy)-(lower alkyl) (lower alkyl)amino-(lower alkyl) or (lower alkyl)thio-(lower alkyl), each of which may be further substituted with one or more substituent (s) selected from the group consisting of heterocyclyl, (lower alkoxy)carbonyl, carboxy and amidated carboxy;
(13 ) R² is aryl which may be substituted with lower alkyl, lower alkenyl, lower alkoxy, (lower alkyl)amino, (lower alkyl)thio, carboxy, (lower alkoxy)carbonyl, (lower alkoxy)-(lower alkyl), (lower alkyl)amino-(lower alkyl) or (lower alkyl)thio-(lower alkyl), each of which may be further substituted with one or more substituent (s) selected from the group consisting of (lower alkoxy)carbonyl, carboxy and carbamoyl;
(14 ) R² is phenyl which may be substituted with (C1-C4)alkyl, (C2-C4)alkenyl, (C1-C4)alkoxy or (C1-C4)amino, each of which may be further substituted with one or more substituent(s) selected from the group consisting of (lower alkoxy)carbonyl, carboxy and carbamoyl;
(15) R² is phenyl which may be substituted with (C1-C4)alkyl, (C2-C4)alkenyl or (C1-C4)alkoxy, each of which may be further substituted with carboxy;
(16) R³ is -Q-R⁷, wherein Q is —CO—, R⁷ is (a) lower alkyl which may be substituted with one or more substituent(s) selected from the group consisting of cycloalkyl, aryl which may be further substituted with aryl(s), and heterocyclyl, (b) lower alkenyl which may be substituted with one or more substituent(s) selected from the group consisting of aryl and heterocyclyl, (c) cycloalkyl, (d) aryl which may be substituted with one or more substituent(s) selected from the group consisting of lower alkyl, aryl which may be further substituted with hydroxy (s), lower alkoxy, aryloxy, hydroxy, and halogen, (e) heterocyclyl which may be substituted with one or more substituent(s) selected from the group consisting of lower alkyl, aryl which may be further substituted with halogen(s), and halogen, (f) aryloxy, or (g) amino which may be substituted with aryl (s) which may be substituted with one or more substituent(s) selected from the group consisting of aryl and heterocyclyl;
(17 ) R³ is -Q-R⁷, wherein Q is —CO—, R⁷ is (d) aryl which may be substituted with one or more substituent(s) selected from the group consisting of lower alkyl, aryl which may be further substituted with hydroxy(s), lower alkoxy, aryloxy, hydroxy, and halogen, (e) heteroaryl which may be substituted with one or more substituent(s) selected from the group consisting of lower alkyl, aryl which may be further substituted with halogen(s), and halogen;
(18) R³ is -Q-R^{7,} wherein Q is —CO—, R⁷ is heteroaryl which may be substituted with one or more substituent(s) selected from the group consisting of lower alkyl, aryl which may be further substituted with halogen(s), and halogen;
(19) R³ is -Q-R⁷, wherein Q is —CO—, R⁷ is nitrogen atom containing condensated heteroaryl or nitrogen atom containing monocyclic heteroaryl which may be substituted with one or more substituent(s) selected from the group consisting of lower alkyl and halogen;
(20) R³ is -Q-R⁷, wherein Q is —CO—, R⁷ is nitrogen atom containing condensated heteroaryl which may be substituted with one or more substituent(s) selected from the group consisting of (C1-C4) alkyl;.
(21) R³ is -Q-R⁷, wherein Q is —CO—, R⁷ is oxygen atom containing condensated heteroaryl or oxygen atom containing monocyclic heteroaryl which may be substituted with one or more substituent(s) selected from the group consisting of lower alkyl and halogen;
(22) R³ is -Q-R⁷, wherein Q is —CO—, R⁷ is oxygen atom containing condensated heteroaryl which may be substituted with one or more substituent(s) selected from the group consisting of (C1-C4) alkyl;
(23) R⁵ is hydrogen or (C1-C4)alkyl;
(24) R⁵ is hydrogen;
(25) R⁶ is hydrogen or (C1-C4)alkyl;
(26) R⁶ is hydrogen.

The Compound (I) is preferably selected from:
sodium 6-{(2S)-2-[(1-benzofuran-2-yl-carbonyl)-amino]-5-[benzyloxycarbonylamino]pentanoylamino}-hexanoate,
(2E)-3-{2-[2S)-2-[(1H-indol-2-ylcarbonyl)amino]-5-[benzyloxycarbonylamino]pentanoylamino]phenyl}-acrylic acid,
(2E)-3-{2-[(2S)-2-[(1-methyl-1H-indol-2-yl-carbonyl)amino]-5-[benzyloxycarbonylamino]-pentanoylamino]phenyl}acrylic acid,
3-{2-[(2S)-2-[(1-methyl-1H-indol-2-ylcarbonyl)-amino]-5-[benzyloxycarbonylamino]pentanoylamino]-phenyl}propanoic acid,
sodium 3-{2-[(2S)-2-[(2-quinolinylcarbonyl)amino]-5-[benzyloxycarbonylamino]pentanoylamino]phenyl}-propanoate,
6-[((2S)-2-[(1-benzofuran-2-ylcarbonyl)amino]-5-{[(benzyloxy)carbonyl]amino}pentanoyl)amino]-2-naphthoic acid,
3-{2-[((2S)-5-{[(benzyloxy)carbonyl]amino}-2-{[(8-methylimidazo[1,2-a]pyridin-2-yl)carbonyl]amino}-pentanoyl)amino]phenyl}propanoic acid,
3-[2-({(2S)-5-{[(benzyloxy)carbonyl]amino}-2-[(2-quinolinylmethyl)amino]pentanoyl}amino)-phenyl]propanoic acid, and
3-[2-({(2S)-5-{[(benzyloxy)carbonyl]amino}-2-[(1H-indol-2-ylcarbonyl)amino]pentanoyl}amino)phenyl]-propanoic acid.

The processes for preparing Compound (I) of the present invention, especially the typical compounds (Ia) and (Ib), are explained in the following processes 1-1 to 2. 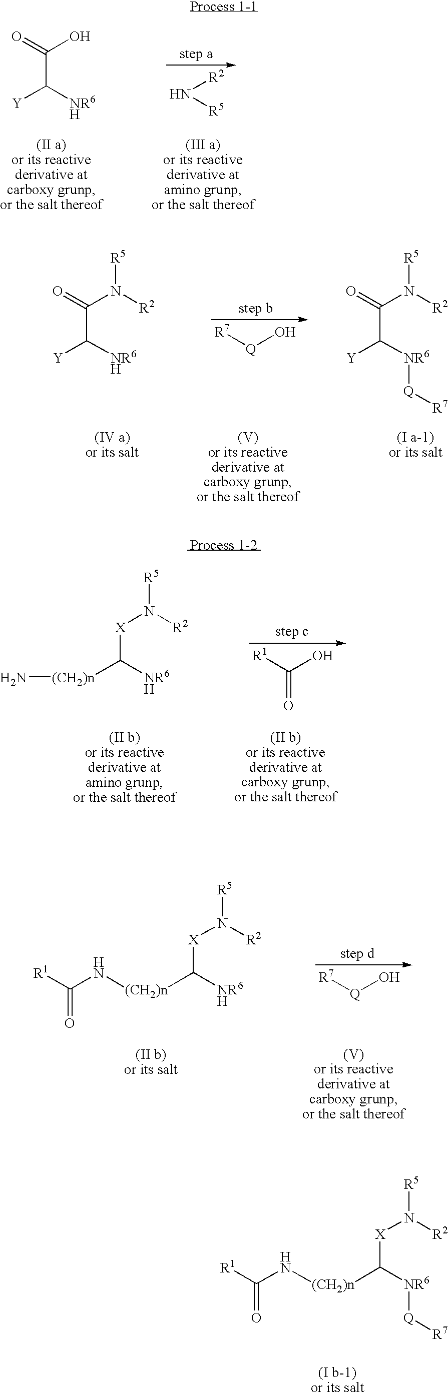 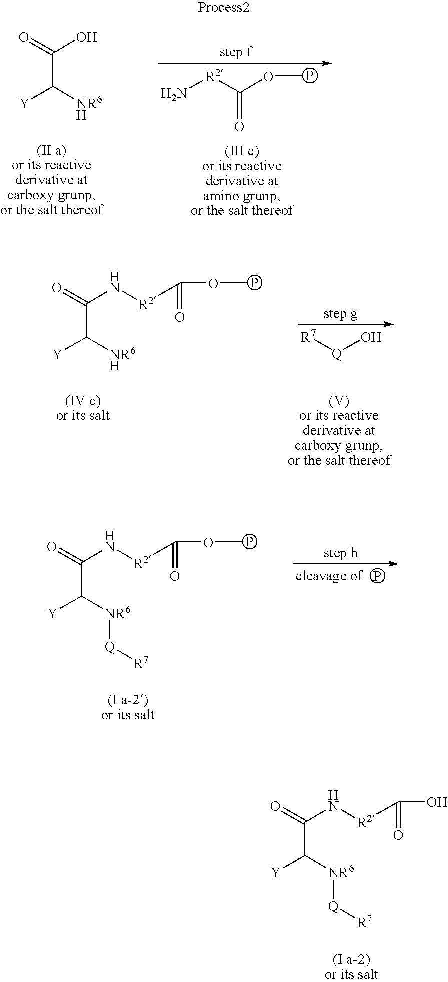
[wherein R¹, R² R³, R⁴, R⁵, R⁶, R⁷, Q, X, Y, Z and n are each as defined above; and
R^{2′} is (1) lower alkyl, (lower alkyl) thio-(lower alkyl) or aryl-(lower alkyl); or
   (2) aryl which may be substituted with lower alkyl, lower alkenyl, aryl, lower alkoxy, (lower alkyl) amino, (lower alkyl)thio, (lower alkoxy)-(lower alkyl), (lower alkyl)amino-(lower alkyl), or (lower alkyl)thio]-(lower alkyl).]
      Process 1-1

The compound (Ia-1) or its salt can be prepared by the following steps:
[step a] reacting the compound (IIa) or its reactive derivative at the carboxy group, or the salt thereof, with the compound (IIIa) or its reactive derivative at the amino group, or the salt thereof to give the compound (IVa) or its salt; and
[step b] reacting the obtained compound (IVa) or its salt, with the compound (V) or its reactive derivative at the carboxy group (in case of Q is —CO—)/the sulfo group (in case of Q is —SO₂—), or the salt thereof.
   [Step a] in Process 1-1

In this process, the amine compound (IIIa) can be used on sale or can be synthesized according to general methods obvious to the person skilled in the organic chemistry from commercial compounds.

Suitable reactive derivative of the amine compound (IIIa) may include Schiff's base type imino or its tautomeric enamine type isomer formed by the reaction of the compound (IIIa) with a carbonyl compound such as aldehyde, ketone or the like; a silyl derivative formed by the reaction of the compound (IIIa) with a silylating reagent such as N,O-bis(trimethylsilyl)acetamide, N-trimethyl-silylacetamide, or the like.

Suitable reactive derivative of the carboxylic acid compound (IIa) may include an acyl halide (carbonyl chloride, carbonyl bromide, and the like.) an acid anhydride, an acid activated amide, an activated ester, or the like.

Suitable acid anhydride may be a symmetric anhydride or a mixed acid anhydride with an acid such as substituted phosphoric acid (e.g., dialkylphosphoric acid, phenylphosphoric acid, diphenylphosphoric acid, dibenzylphosphoric acid, halogenated phosphoric acid), dialkylphosphorous acid, sulfuric acid, thiosulfuric acid, alkanesulfonic acid (e.g., methanesulfonic acid, ethanesulfonic acid), alkylcarbonic acid, aliphatic carboxylic acid (e.g., pivalic acid, pentanoic acid, isopentanoic acid); aromatic carboxylic acid (e.g., benzoic acid, chlorobenzoic acid, fluorobenzoic acid, nitrobenzoic acid), or the like.

Suitable activated amide may be imidazolylamide, 4-substituted imidazolylamide, dimethylpyrazolyl-amide, triazolylamide, tetrazolylamide, or the like.

Suitable activated ester may be dimethyliminomethyl [(CH₃)₂N⁺═CH—] ester, vinyl ester, propargyl ester, 4-nitrophenyl ester, 2,4-dinitrophenyl ester, trichlorophenyl ester, pentachlorophenyl ester, pentafluorophenyl ester, methanesulfonylphenyl ester, phenyl thioester, p-nitrophenyl thioester, carboxymethyl thioester, pyranyl ester, pyridyl ester, 8-quinolyl thioester, an activated ester with a N-hydroxy compound (e.g., N,N-dimethylhydroxylamine, 1-hydroxy-2H-pyridone, N-hydroxysuccinimide, N-hydroxybenzotrioxazole, N-hydroxyphthalimide,), or the like.

When the carboxylic acid compound (IIa) is used in free acid form or its salt form in the reaction, the reaction is preferably carried out in the presence of condensing agent.

Suitable condensing agent may include a carbodiimide [e.g., N,N′-diisopropylcarbodiimide (DIPCI), N,N′-dicyclohexylcarbodiimide (DCC) N-cyclohexyl-N′-(4-diethylaminocyclohexyl)-carbodiimide, N-ethyl-N′-(3-dimethylaminopropyl)-carbodiimide or its hydrochloride], diphenylphosphinic azido, diphenylphosphinic chloride, diethylphosphoryl cyanide, bis (2-oxo-3-oxazolidinyl)phosphinic chloride, N,N′-carbonyldiimidoxazole, 2-ethoxy-1-ethoxycarbonyl-1,2-dihydroquinoline, cyanuric chloride, or the like.

The reaction maybe also carried out in the presence of organic or inorganic base such as alkali metal carbonate, tri(lower)alkylamine, pyridine, N-(lower)alkylmorphorine, or the like.

The reaction is usually carried out in a conventional solvent such as water, acetone, alcohol [e.g., methanol, ethanol, isopropyl alcohol, or the like], THF, dioxane, toluene, methylene chloride, chloroform, DMF or any other organic solvents which do not adversely affect the reaction, or the mixture thereof.

The reaction temperature is not limited and the reaction is usually carried out under cooling to warming.

For example, this reaction can be referred to that of Example 27-1 described later.

[Step b] in Process 1-1

(i) in case where Q is —CO—

Suitable reactive derivative of the carboxy compound (V), the condensing agent, base, solvent employable in this process and the reaction temperature are the same as explained above.

This reaction can be referred to that of Example 27-3.

(ii) in case where Q is —SO₂—

Suitable reagent to be used in the sulfonylation is, for example, sulfonyl chloride, sulfonic anhydride (e.g., trifluoromethanesulfonic anhydride) or the like. This reaction is preferably carried out in the presence of base.

Suitable base may include the inorganic base such as alkali metal hydroxide (e.g., sodium hydroxide, potassium hydroxide), alkaline earth metal hydroxide (e.g., magnesium hydroxide, calcium hydroxide), alkali metal carbonate (e.g., sodium carbonate, potassium carbonate), alkaline earth metal carbonate (e.g., magnesium carbonate calcium carbonate) or the like; and the organic base such as tri (lower) alkylamine {e.g., trimethylamine, diisopropylethylamine (DIPEA)}, pyridine, or the like.

This reaction is usually carried out in a conventional solvent such as toluene, acetonitrile, benzene, DMF, THF, methylene chloride, ethylene chloride, chloroform, or any other organic solvent which does not adversely affect the reaction.

The reaction temperature is not limited and the reaction is usually carried out under cooling to warming.

Process 1-2

The compound (Ib-1) or its salt can be prepared by the following steps:
(i) reacting the compound (IIb) or its reactive derivative at the amino group, or the salt thereof, with the compound (IIIb) or its reactive derivative at the carboxy group, or the salt thereof to give the compound (IVb) or its salt [step c]; and
(ii) reacting the compound (IVb) or its salt, with the compound (V) or its reactive derivative at the carboxy group (in case of Q is —CO—)/the sulfo group (in case of Q is —SO₂—), or the salt thereof [step d].
   [Step c] in Process 1-2

In this process, the compound (IIb) can be obtained in a similar mariner to that of [step b] in Process 1-1.

This reaction can be referred to that of Example 36-2 described later.

[Step d] in Process 1-2

In this process, the compound (Ib-1) can be obtained in a similar manner to that of [step b] in Process 1-1.

This reaction can be referred to that of Example 27-3 described later.

Process 2

In addition, the compound (I) may be obtained on a solid phase support linkage illustrated above.

For example, the compound (Ia-2) or its salt can be prepared by the following steps:
(i) preparing the resin-bound amine compound (IIIc) [step e];
(ii) reacting the carboxylic acid compound (IIa) or 30 its reactive derivative at the carboxy group, or the salt thereof, with the above resin-bound amine compound (IIIc) or its reactive derivative at the amino group, or the salt thereof to give the amine compound (IVc) or its salt [step f];
(iii) reacting the amine compound (IVc) or its salt, with the compound (V) or its reactive derivative at the carboxy group (in case of Q is —CO—)/the sulfo group (in case of Q is —SO₂—), or the salt thereof [step g]; and
(iv) a cleavage reaction of the resin [step h].
   [Step e] in Process 2

The resin-bound amine compound (IIIc) is coupled to a solid support such as trytyl-resin by treatment with an activating agent, conveniently 4-nitrophenyl chloroformate in the presence of base such as DIPEA in a solvent such as THF, DMF, dichloromethane, or their mixture.

This reaction can be referred to that of Example 1 described later.

[Step f] and [Step g] in Process 2

In these processes, the compounds (IVc) and (Ia-2′) can be obtained in a similar manner to that of [step b] in Process 1-1.

This reaction can be referred to that of Examples 1 and 27-3.

[Step h] in Process 2

Cleavage from the resin is effected, in the case of trytyl resin, by treatment with acid such as trifluoroacetic acid (TFA) as mixture with dichloromethane, or the like.

This reaction can be referred to that of Example 1.

Above processes, all starting materials and product compounds maybe salts. The compounds of above processes can be converted to salt according to a conventional method.

In the above compounds, which have reactive group, may be protected at the group on cue and be deprotected on cue. In these reactions (protecting or deprotecting steps), concerning the kind of protective group and the condition of the reaction, ┌PROTECTIVE GROUPS IN ORGANIC SYNTHESIS Second Edition┘ T. W. Green and P. G. M. Wuts, John Wiley & Sons, INC. (the contents of which are hereby incorporated by reference) may be referred.

The patents, patent applications and publications cited herein are incorporated by reference.

For therapeutic purpose, Compound (I) and a pharmaceutically acceptable salt thereof of the present invention can be used in a form of pharmaceutical preparation containing at least one of said compound as an active ingredient, in admixture with a pharmaceutically acceptable carrier.

The pharmaceutically acceptable carrier can be exemplified by excipient (e.g., sucrose, starch, mannit, sorbit, lactose, glucose, cellulose, talc, calcium phosphate, calcium carbonate), binding agent (e.g., cellulose, methyl cellulose, hydroxypropylcellulose, polypropylpyrrolidone, gelatin, gum arabic, polyethyleneglycol, sucrose, starch), disintegrator (e.g., starch, carboxymethyl cellulose, calcium salt of carboxymethyl cellulose, hydroxypropylstarch, sodium glycol-starch, sodium bicarbonate, calcium phosphate, calcium citrate), lubricant (e.g., magnesium stearate, talc, sodium laurylsulfate), flavoring agent (e.g., citric acid, mentol, glycine, orange powders), preservative (e.g., sodium benzoate, sodium bisulfite, methylparaben, propylparaben), stabilizer (e.g., citric acid, sodium citrate, acetic acid), suspending agent (e.g., methyl cellulose, polyvinylpyrrolidone, aluminum stearate, etc.), dispersing agent, aqueousdilutingagent (e.g., water), base wax (e.g., cacao butter, polyethylene-glycol, white petrolatum).

Such a pharmaceutical composition of the present invention can be used in the form of a pharmaceutical preparation, for example, in solid, semisolid or liquid form (e.g., tablet, pellet, troche, capsule, suppository, cream, ointment, aerosol, powder, solution, emulsion, suspension, or the like), which contains Compound (I) orapharmaceutic allyacceptable salt thereof as an active ingredient, suitable for rectal, pulmonary (nasal or buccal inhalation), nasal, ocular, external (topical), oral or parenteral (including subcutaneous, intravenous and intramuscular) administrations or insufflation.

The pharmaceutical preparations of the present invention may be capsules, tablets, dragees, granules, inhalant, suppositories, solution, lotion, suspension, emulsion, ointment, gel, cream, or the like. If desired, there may be included in these preparations, auxiliary substances, stabilizing agents, wetting or emulsifying agents, buffers and other commonly used additives.

While the dosage of therapeutically effective amount of the Compound (I) depend upon the age and condition of each individual patient, an average single dose of about 0.01 mg, 0.1 mg, 1 mg, 10 mg, 50 mg, 100 mg, 250 mg, 500 mg and 1000 mg of the Compound (I) may be effective for treating the above-mentioned diseases. In general, amounts between 0.01 mg/kg and about 50 mg/kg, 1 to 4 times per day may be administered.

This application is based on Australian Patent Application No. 2003907110 filed on Dec. 22, 2003, the contents of which are hereby incorporated by references.

Although the present invention has been fully described by way of example, it is to be understood that various changes, and modifications will be apparent to those skilled in the art. Therefore, unless otherwise such changes and modifications depart from the scope of the present invention hereinafter defined, they should be construed as being included therein.

### THE BEST MODE FOR CARRYING OUT THE INVENTION

The following Examples are given only for the purpose of illustrating the present invention in more detail.

Although the present invention has been fully described by way of example, it is to be understood that various changes and modifications will be apparent to those skilled in the art. Therefore, unless such changes and modifications depart from the objective of the present invention, they should be construed as being included therein.

Abbreviations used in this application are as follows:
EtOAc: ethyl acetate
DMF: N,N-dimethylformamide
Boc: tert-butoxycarbonyl
Fmoc: 9-fluorenylmethoxycarbonyl
WSCD: 1-(3-dimethylaminopropyl)-3-ethyl-carbodiimide hydrochloride
DIPCI: 1,3-diisopropylcarbodiimide
TBTU: O-benzotriazole-N,N,N,N′-tetramethyl-uronium-hexafluorophosphate
HOBT: 1-hydroxybenzotriazole
THF: tetrahydrofuran
DIPEA: N,N-diisopropylethylamirie
EtOH: ethanol
5 MeOH: methanol
NMP: 1-methyl-2-pyrrolidinone
BSA: N,O-bis(trimethylsilyl)acetamide
PyBOP: benzotriazole-1-yl-oxy-tris-pyrrolidino-phosphonium hexafluorophosphate
DIEA: N,N-diisopropylethylamine
DMSO: dimethyl sulfoxide
DEAD: diethyl azodicarboxylate
DCM: dichloromethane
Et₂O: diethyl ether
PyBroP: bromo-tris-pyrrolidino-phosphonium hexafluorophosphate
TFA: trifluoroacetic acid
MSNT: 1-(mesitylene-3-sulfonyl)-3-nitro-1H-1,2,4-triazole
Et₂O: diethyl ether
Ac₂O: acetic anhydride
HATU: O-(7-azabenzotriazol-1-yl)-1,1,3,3-tetramethyluronium hexafluorophosphate
TISH: triisopropylsilane
Fmoc: 9-fluorenylmethoxycarbonyl
Mtt: (4-methyl)trityl
HPLC: high performance liquid chromatography

### EXAMPLE 1

### 6-{(2S) -2-[(1-Benzofuran-2-ylcarbonyl)amino]-5-[benzyloxycarbonylamino]pentanoylamino}hexanoic acid

A solution of 6-[9-(flouorenylmethoxycarbonyl)-amino]hexanoic acid (180 mg) and DIPEA (0.12 mL) in dichloromethane (3 mL) was added to a reaction vessel containing Cl-trytyl resin (200 mg, 1.3 mmol/g, loading). After the vessel was shaken for 12 hours at room temperature, the resin was washed successively with dichloromethane, THF, DMF and dichloromethane.

After cleavage of Fmoc by using 20% piperazine in DMF (5 mL), 2-Fmoc-5-[benzyloxycarbonylamino]-pentanoic acid (254 mg), TBTU (170 mg), HOBT (70 mg) and DIPEA (0.18 mL) were added to a solution of the obtained resin in DMF (3 mL). After the vessel was shaken for 12 hours at room temperature, the resin was washed successively with dichloromethane, THF, DMF and dichloromethane.

After cleavage 9-(flouorenylmethoxy carbonyl)amide by using 20% piperazine in DMF (5 mL), benzofuran-2-carboxylic acid (210 mg), DIPCI (0.21 mL) and DIPEA (0.23 mL) were added successively to a solution of the obtained resin in dichloromethane (3 mL) After the vessel was shaken for 12 hours at room temperature, the resin was washed successively with dichloromethane, THF, DMF, and dichloromethane.

Cleavage from the resin was performed with 1% trifluoromethanesulfonic acid in dichloromethane (5 mL) for 10 minutes at room temperature. After the filtrated solvent was evaporated under pressure, the residue was washed with ether to give the target compound (100 mg, 72%).

MS: 524 (M+1).

### EXAMPLE 2

### {(2S)-2-[(1-Benzofuran-2-ylcarbonyl)amino]-5-[benzyloxycarbonylamino]pentanoylamino}acetic acid

The target compound was obtained in a similar manner to that of Example 1.

MS: 468 (M+1).

### EXAMPLE 3

### 4-{(2S)-2-[(1-Benzofuran-2-ylcarbonyl)amino]-5-[benzyloxycarbonylamino]pentanoylamino}butanoic acid

The target compound was obtained in a similar manner to that of Example 1.

MS: 496 (M+1).

### EXAMPLE 4

### 5-{(2S)-2-[(1-Benzofuran-2-ylcarbonyl)amino]-5-[benzyloxycarbonylamino]pentanoylamino}pentanoic acid

The target compound was obtained in a similar manner to that of Example 1.

MS: 510 (M+1).

### EXAMPLE 5

### 7-{(2S)-2-[(1-Benzofuran-2-ylcarbonyl)amino]-5-[benzyloxycarbonylamino]pentanoylamino}heptanoic acid

The target compound was obtained in a similar manner to that of Example 1.

MS: 538 (M+1).

### EXAMPLE 6

### 6-{(2S)-2-[(1-Benzofuran-2-ylcarbonyl)amino]-3-[benzyloxycarbonylamino]propanoylamino}hexanoic acid

The target compound was obtained in a similar manner to that of Example 1.

MS: 496 (M+1).

### EXAMPLE 7

### 6-{(2S)-2-[(1-Benzofuran-2-ylcarbonyl)amino]-4-[benzyloxycarbonylamino]butanoylamino}hexanoic acid

The target compound was obtained in a similar manner to that of Example 1.

MS: 510 (M+1).

### EXAMPLE 8

### 6-{(2S)-2-[(1-Benzofuran-2-ylcarbonyl)amino]-6-[benzyloxycarbonylamino]hexanoylamino}hexanoic acid

The target compound was obtained in a similar manner to that of Example 1.

MS: 538 (M+1).

### EXAMPLE 9

### 6-{(2R)-2-[(1-Benzofuran-2-ylcarbonyl)amino]-6-[benzyloxycarbonylamino]hexanoylamino}hexanoic acid

The target compound was obtained in a similar manner to that of Example 1.

MS: 538 (M+1).

### EXAMPLE 10

### 6-{(2S)-2-[(1-Benzofuran-2-ylcarbonyl)amino]-3-phenylpropanoylamino}hexanoic acid

The target compound was obtained in a similar manner to that of Example 1.

MS: 423 (M+1).

### EXAMPLE 11

### 6-{(2S)-2-[(1-Benzofuran-2-ylcarbonyl)amino]-3-methylbutanoylamino}hexanoic acid

The target compound was obtained in a similar manner to that of Example 1.

MS: 375 (M+1).

### EXAMPLE 12

### 6-[(2S)-1-(1-Benzofuran-2-ylcarbonyl)-2-(pyrrolidinyl)carbonylamino]hexanoic acid

The target compound was obtained in a similar manner to that of Example 1.

MS: 373 (M+1).

### EXAMPLE 13

### 6-{(2S)-2-[(1-Benzofuran-2-ylcarbonyl)amino]-5-[ethoxycarbonylamino]pentanoylamino}hexanoic acid

The target compound was obtained in a similar manner to that of Example 1.

MS: 476 (M+1).

### EXAMPLE 14

### 6-{(2S)-2-[(1-Benzofuran-2-ylcarbonyl)amino]-5-[benzoylamino]pentanoylamino}hexanoic acid

The target compound was obtained in a similar manner to that of Example 1.

MS: 494 (M+1).

### EXAMPLE 15

### 6-{(2S)-2,5-Bis[(1-benzofuran-2-ylcarbonyl)amino]-pentanoylamino}hexanoic acid

The target compound was obtained in a similar manner to that of Example 1.

MS: 534 (M+1).

### EXAMPLE 16

### 6-{(2S)-2-[(1-Benzothien-2-ylcarbonyl)amino]-5-[benzyloxycarbonylamino]pentanoylamino}hexanoic acid

The target compound was obtained in a similar manner to that of Example 1.

MS: 540 (M+1).

### EXAMPLE 17

### 6-{(2S)-2-[(2E)-(3-Phenyl-2-propenoyl)amino]-5-[benzyloxycarbonylamino]pentanoylamino}hexanoic acid

The target compound was obtained in a similar manner to that of Example 1.

MS: 510 (M+1).

### EXAMPLE 18

### 6-{(2S)-2-[(4-Biphenylylcarbonyl)amino]-5-[benzyl-oxycarbonylamino]pentanoylamino}hexanoic acid

The target compound was obtained in a similar manner to that of Example 1.

MS: 560 (M+1).

### EXAMPLE 19

### 6-{(2S)-2-[(2-Naphthoyl)amino]-5-[benzyloxy-carbonylamino]pentanoylamino}hexanoic acid

The target compound was obtained in a similar manner to that of Example 1.

MS: 534 (M+1).

### EXAMPLE 20

### 6-{(2S)-2-[(1H-Indol-2-ylcarbonyl)amino]-5-[benzyloxycarbonylamino]pentanoylamino}hexanoic acid

The target compound was obtained in a similar manner to that of Example 1.

MS: 523 (M+1).

### EXAMPLE 21

### 6-{(2S)-2-[(1H-Indol-3-ylcarbonyl)-amino]-5-[benzyloxycarbonylamino]pentanoylamino}-hexanoic acid

The target compound was obtained in a similar manner to that of Example 1.

MS: 523 (M+1).

### EXAMPLE 22

### 6-{(2S)-2-[(1H-Indol-6-ylcarbonyl)amino]-5-[benzyloxycarbonylamino]pentanoylamino}hexanoic acid

The target compound was obtained in a similar manner to that of Example 1.

MS: 523 (M+1).

### EXAMPLE 23

### Sodium 6-{(2S)-2-[(1-benzofuran-2-yl-carbonyl)-amino]-5-[benzyloxycarbonylamino]pentanoylamino}-hexanoate

To a solution of 6-{2S)-2-[(1-benzofuran-2-yl-carbonyl)amino]-5-[benzyloxycarbonylamino]-pentanoylamino}hexanoic acid (50 mg) obtained in Example 1 in MeOH, was added 1N NaOH (0.1 mL) at room temperature. After the solvent was evaporated under pressure, the residue was washed with ether to give the target compound (50 mg).

MS: 524 (M+1). ¹H-NMR (200 MHz, DMSO-d₆): δ 1.2-1.8 (10H, m), 1.95 (2H, t, J=7.0 Hz), 3.03 (4H, t, J=6.2 Hz), 4.43 (1H, m), 4.99 (2H s), 7.2-7.6 (8H, m), 7.6-7.9 (3H, m), 8.31 (1H, t, J=5.4 Hz), 8.87 (1H d, J=8.2 Hz).

### EXAMPLE 24

### Benzyl N-{(4S)-4-(1-benzofuran-2-yl-carbonyl)-amino]-5-oxo-5-[(6-oxo-6-benzylaminohexyl)amino]-pentyl}carbamate

To a solution of 6-{(2S)-2-(1-benzofuran-2-ylcarbonyl)amino]-5-[benzyloxycarbonylamino]-pentanoylamino}hexanoic acid (50 mg) obtained in Example 1 in DMF (1 mL), were added successively TBTU (84 mg), HOBT (18 mg), DIPEA (0.023 mL) and benzylamine (0.014 mL) at room temperature. After stirring for 4 hours, the mixture was diluted with EtOAc. The solution was washed successively with water, 1N HCl, 1N NaOH and brine, and dried over MgSO₄. After the filtrated solvent was evaporated under pressure, the residue was washed with ether to give the target compound (40 mg).

MS: 613 (M+1).

### EXAMPLE 25

### Benzyl N-{(4S)-4-[(1-benzofuran-2-ylcarbonyl)-amino]-5-oxo-5-[6-oxo-6-[(2-phenylethylamino-hexyl)amino]pentyl]carbamate

The target compound was obtained in a similar manner to that of Example 24.

MS: 627 (M+1).

### EXAMPLE 26

### Benzyl N-{(4S)-4-[(1-benzofuran-2-ylcarbonyl)-amino]-5-oxo-5-[6-oxo-6-[(3-phenylpropylamino-hexyl)amino]pentyl]carbamate

The target compound was obtained in a similar manner to that of Example 24.

MS: 641 (M+1).

### EXAMPLE 27-1

### Methyl(2E)-3-{2-[(2)-5-[benzyloxycarbonylamino]-2-[tert-butoxycarbonylamino]pentanoylamino]-phenyl}acrylate

To a solution of (2S)-2-(tert-butoxycarbonylamino)-5-(benzyloxycarbonylamino)-pentanoic acid (6.00 g) and methyl (2E)-3-(2-aminophenyl)acrylate (3.77 g) in DMF (60 mL), were added successively HOBT (3.32 g), WSCD (6.28 g) and 4-(dimethylamino)pyridine (400 mg). The mixture was stirred at 50° C. for 15 hours.

After cooling to room temperature, the mixture was quenched by the addition of water (120 mL) and extracted with EtOAc (120 mL). The extract was washed successively with water (120 mL), saturated aqueous sodium hydrogencarbonate (120 mL), 1N HCl (120 mL), water (120 mL) and brine (120 mL), and dried over MgSO₄. Filtration followed by evaporation gave a crude product which was chromatographed on silica gel (eluent: hexane/EtOAc=1/1) to give the target compound (2.58 g) as a yellow crystalline solid.

MS ((+)ESI) m/z: 548 (M+Na)⁺.

### EXAMPLE 27-2

### Methyl(2E)-3-{2-(2S)-2-amino-5-[benzyloxycarbonylamino]pentanoylamino]phenyl}acrylate hydrochloride

To a suspension of methyl (2E)-3-{2-[(2S)-2-[tert-butoxycarbonylamino]-5-[benzyloxycarbonylamino]pentanoylamino]phenyl}-acrylate (2.58 g) obtained in Example 27-1 in EtOAc (20 mL), was added 4N hydrogen chloride in EtOAc (20 mL). The mixture was stirred at room temperature for 1 hour. The solvent was removed by evaporation to give the target compound (2.40 g) as a yellow solid.

MS ((+)ESI) m/z: 426 (M+H)⁺, 448 (M+Na)⁺.

### EXAMPLE 27-3

### Methyl(2E)-3-{2-[(2S)-2-[(1H-indol-2-ylcarbonyl)-amino]-5-[benzyloxycarbonylamino]pentanoylamino]-phenyl}acrylate

To a solution o f methyl (2E)-3-{2-[(2S)-2-amino-5-[benzyloxycarbonyl-amino]pentanoylamino]phenyl}acrylate hydrochloride (400 mg) obtained in Example 27-2 in DMF (4.0 mL), were added successively indole-2-carboxylic acid (154 mg), HOBT (176 mg) and WSCD (0.32 mL). The mixture was stirred at room temperature for 16 hours. The mixture was diluted with EtOAc (10 mL) and washed with water (10 mL×2). The organic layer was stirred vigorously at room temperature for 1 hour. The precipitates were collected by filtration, washed with EtOAc (1 mL×2), and dried under reduced pressure to give the target compound (115 mg) as a white solid.

MS ((+)ESI) m/z: 591 (M+Na)⁺.

### EXAMPLE 28

### (2E)-3-{2-(2S)-2-[(1H-indol-2-yl carbonyl)amino]-5-[benzyloxycarbonylamino]pentanoylamino]phenyl}-acrylic acid

To a suspension of methyl (2E)-3-{2-[(2S)-2-[(1H-indol-2-yl carbonyl)amino]-5-[benzyloxycarbonylamino]pentanoylamino]phenyl}-acrylate (109 mg) obtained in Example 27-3 in MeOH (2.0 mL) and THF (2.0 mL), was added 1N NaOH (0.38 mL) The mixture was refluxed for 2 hours. After cooling to room temperature, the mixture was quenched by the addition of 1N HCl (20 mL) and extracted with EtOAc (20 mL). The extract was washed with water (20 mL) and brine (20 mL), and dried over MgSO₄. Filtration followed by evaporation gave the target compound (102 mg) as a pale yellow solid.

MS ((−)ESI) m/z: 553 (M−H)⁻.

¹H-NMR (200 MHz, DMSO-d₆):δ 1.61-1.99 (4H, m), 3.05-3.11 (2H, m), 4.63-4.79 (1H, m), 5.01 (2H, s), 6.49 (1H, d, J=15.9 Hz), 7.00-7.83 (16H, m), 8.61 (1H, d, J=7.7 Hz), 10.0 (1H, br-s), 11.6 (1H, br-s),12.9 (1H, br-s)

### EXAMPLE 29

### Methyl(2E)-3-{2-[(1-methyl-1H-indol-2-ylcarbonyl)amino]-5-[benzyloxycarbonylamino]-pentanoylamino]phenyl}acrylate

The target compound was obtained in a similar manner to that of Example 27-3.

MS ((+) ESI) m/z: 605 (M+Na)⁺.

### EXAMPLE 30

### 5(2E)-3-{2-[(2S)-2-[(1-Methyl-1H-indol-2-yl-carbonyl)amino]-5-[benzyloxycarbonylamino]-pentanoylamino]phenyl}acrylic acid

The target compound was obtained in a similar manner to that of Example 28.

MS ((−)ESI) m/z: 567 (M−H)⁻.

¹H-NMR (200 MHz, DMSO-d₆): δ 1.61-1.99 (4H, m), 3.09-3.11 (2H, m), 3.99 (3H, s), 4.60-4.71 (1H, m), 5.01 (2H, s), 6.49 (1H, d, J=15.9 Hz),7.07-7.84 (16H, m), 8.62 (1H, d, J=7.7 Hz), 9.97 (1H, br-s), 12.4 (1H, br-s).

### EXAMPLE 31

### Methyl(2E)-3-{2-(2S)-2-(4-biphenylylcarbonyl)-amino]-5-[benzyloxycarbonylamino]pentanoylamino]-phenyl}acrylate

The target compound was obtained in a similar manner to that of Example 27-3.

MS ((+)ESI) m/z: 628 (M+Na)⁺.

### EXAMPLE 32

### (2E)-3-{2-[(2S)-2-(4-Biphenylylcarbonyl)amino]-5-[benzyloxycarbonylamino]pentanoylamino]phenyl}-acrylic acid

The target compound was obtained in a similar manner to that of Example 28.

MS ((−)ESI) m/z: 590 (M−H)⁻.

¹H-NMR (200 MHz, DMSO-d₆): δ 1.60-1.99 (4H, m), 3.08-3.11 (2H, m), 4.64-4.79 (1H, m), 5.01 (2H, s), 6.48 (1H, d, J=15.9 Hz), 7.19-7.54 (12H, m), 7.73-7.83 (6H, m), 8.04 (2H, d, J=8.4 Hz), 8.66 (1H, d, J=7.5 Hz), 9.97 (1H, br-s), 12.4 (1H, br-s).

### EXAMPLE 33

### Methyl(2E)-3-{2-[(2S)-2-[(1-benzofuran-2-yl-carbonyl)amino]-5-[benzyloxycarbonylamino]-pentanoylamino]phenyl}acrylate

The target compound was obtained in a similar manner to that of Example 27-3.

MS ((+)ESI) m/z: 592 (M+Na)⁺.

### EXAMPLE 34-1

### Methyl 3-{2-[(2S)-2-[(1-benzofuran-2-ylcarbonyl)-amino]-5-[aminopentanoylamino]phenyl}propanoate

To a solution of methyl (2E)-3-{2-[(2S)-2-[(1-benzofuran-2-ylcarbonyl)amino]-5-[benzyloxy-carbonylamino]pentanoylamino]phenyl}acrylate (1.30 g) obtained in Example 33 in MeOH (26 mL) and THF (26 mL), was added 10% palladium on activated carbon (50% wet, 130 mg). The mixture was hydrogenated (1 atm) at room temperature for 90 minutes. The catalyst was removed by filtration through a Celite cake and washed with MeOH. The filtrate was concentrated in vacuo to give the target compound (1.19 g) as a white solid.

### EXAMPLE 34-2

### Methyl 3-{2-[(2S)-2-[(1-benzofuran-2-ylcarbonyl)-amino]-5-[benzyloxycarbonylamino]pentanoylamino]-phenyl}propanoate

To a solution of methyl 3-{2-[(2 )-2-[(1-benzofuran-2-ylcarbonyl) amino]-5-aminopentanoylamino]phenyl]propanoate (1.05 g) obtained in Example 34-1 in THF (10 mL) and water (10 mL) was added benzyl chloroformate (0.38 mL) at 5° C. while the pH was adjusted to 8.0-9.0 by the addition of 10% aqueous NaOH.

After stirring at the same temperature for 30 minutes, the mixture was extracted with EtOAc (20 mL) The extract was washed with water (20 mL) and brine (20 mL), and dried over MgSO₄. Filtration followed by evaporation gave a crude solid which was purified by silica gel chromatography (eluent: hexane/EtOAc=1/1) and recycling preparative HPLC equipped with a gel permeation chromatography column (eluent: chloroform) to give the target compound (572 mg) as a white crystalline solid.

MS ((+)ESI) m/z: 594 (M+Na)⁺.

### EXAMPLE 35

### 3-{2-[(2S)-2-[(1-Benzofuran-2-ylcarbonyl)amino]-5-[benzyloxycarbonylamino]pentanoylamino]phenyl}-propanoic acid

The target compound was obtained in a similar manner to that of Example 28.

MS ((−)ESI) m/z: 556 (M−H)⁻.

¹H-NMR (200 MHz, DMSO-d₆): δ 1.57-1.99 (4H, m), 2.45-2.51 (2H, m), 2.78-2.85 (2H, m), 3.06-3.09 (2H, m), 4.65-4.68 (1H, m), 5.00 (2H, s), 7.11-7.52 (12H, m), 7.66-7.81 (3H, m), 8.75 (1H, d, J=7.7 Hz), 9.62 (1H, br-s) 12.2 (1H, br-s).

### EXAMPLE 36-1

### Methyl(2E)-3-{2-[(2)-2-[tert-butoxycarbonylamino]-5-amino-pentanoylamino]phenyl}propanoate

The target compound was obtained in a similar manner to that of Example 34-1.

MS ((+)ESI) m/z: 394 (M+H)⁺.

### EXAMPLE 36-2

### Methyl 3-{2-[(2S)-2-[tert-butoxycarbonylamino]-5-[(2-chlorobenzyloxycarbonyl)amino]pentanoylamino]-phenyl}propanoate

To a solution of methyl (2E)-3-{2-[(2S)-2-[tert-butoxycarbonylamino]-5-aminopentanoylamino]phenyl}propanoate (4_{—}34 g) obtained in Example 36-1 in dichloromethane (80 mL), was added triethylamine (2.31 mL). The solution was cooled to 5° C. To the solution was added 2-chlorobenzyl chloroformate (1.86 mL) at 5° C., and the mixture was stirred at the same temperature for 1 hour.

The solvent was removed by evaporation, and the residue was partitioned between 1N HCl (80 mL) and EtOAc (80 mL). The organic layer was separated, washed successively with water (80 mL), saturated aqueous sodium hydrogencarbonate (80 mL) and brine (80 mL), and dried over MgSO₄. Filtration followed by evaporation gave a yellow solid which was chromatographed on silica gel (eluent:hexane/EtOAc=2/1 to 3/2) to give the target compound (3.62 g) as a white solid.

MS ((+)ESI) m/z: 584 (M+Na)⁺.

### EXAMPLE 36-3

### Methyl 3-{2-[(2S)-2-amino-5-[(2-chlorobenzyloxy-carbonyl)amino]pentanoylamino]phenyl}propanoate hydrochloride

To a suspension of methyl 3-{2-[(2S)-2-[tert-butoxycarbonylamino]-5-[(2-chlorobenzyloxy-carbonyl)amino]pentanoylamino]phenyl}propanoate (3.45 g) obtained in Example 36-2 in EtOAc (15 mL), was added 4N hydrogen chloride in EtOAc (45 mL). The mixture was stirred at room temperature for 1 hour. The mixture was concentrated in vacuo to give the target compound (3.11 g) as a pale yellow viscous oil.

MS ((+)ESI) m/z: 462 (M+H)⁺.

### EXAMPLE 36-4

### Methyl 3-{2-[(2S)-2-[(1-benzofuran-2-yl-carbonyl)-amino]-5-[(2-chlorobenzyloxycarbonyl)amino]-pentanoylamino}phenyl]propanoate

The target compound was obtained in a similar manner to that of Example 27-3.

### EXAMPLE 37

### 3-{2-[(2S)-2-[(1-Benzofuran-2-yl-carbonyl) amino]-5-[(2-chlorobenzyloxycarbonyl)amino]pentanoyl-amino]phenyl]propanoic acid

The target compound was obtained in a similar manner to that of Example 28.

MS ((−)ESI) m/z: 590 (M−H)⁻.

¹H-NMR (200 MHz, DMSO-d₆): δ 1.59-1.99 (4H, m) 2.45-2.50 (2H, m), 2.78-2.85 (2H, m), 3.07-3.10 (2H, m) 4.66-4.69 (1H, m), 5.09 (2H, s) 7.11-7.52 (11H, m), 7.66-7.81 (3H, m), 8.74 (1H, d, J=7.6 Hz), 9.61 (1H, br-s), 12.1 (1H, br-s).

### EXAMPLE 38-1

### Methyl 3-{2-[(2S)-2-[tert-butoxycarbonylamino]-5-[(benzyloxycarbonyl)amino]pentanoylamino]phenyl}-propanoate

The target compound was obtained in a similar manner to that of Example 36-2.

MS ((+)ESI) m/z: 550 (M+Na)⁺.

### EXAMPLE 38-2

### Methyl 3-{2-[(2S)-2-amino-5-[benzyloxycarbonyl-amino]pentanoylamino]phenyl}propanoate hydrochloride

The target compound was obtained in a similar manner to that of Example 36-3.

MS ((+)ESI) m/z: 428 (M+H)⁺.

### EXAMPLE 38-3

### Methyl 3-{2-[(2S)-2-[(1-methyl-1H-indol-2-yl-carbonyl)amino]-5-[benzyloxycarbonylamino]-pentanoylamino]phenyl}propanoate

The target compound was obtained in a similar manner to that of Example 27-3.

MS ((+)ESI) m/z: 607 (M+Na)⁺.

### EXAMPLE 39

### 3-{2-[(2S)-2-[(1-Methyl-1H-indol-2-ylcarbonyl)-amino]-5-[benzyloxycarbonylamino]pentanoylamino]-phenyl}propanoic acid

The target compound was obtained in a similar manner to that of Example 28.

MS ((−)ESI) m/z: 569 (M−H)⁻.

¹H-NMR (200 MHz, DMSO-d₆): δ 1.59-1.91 (4H, m), 2.48-2.54 (2H, m), 2.79-2.87 (2H, m), 3.05-3.10 (2H, m), 3.98 (3H, s), 4.55-4.66 (1H, m), 5.01 (2H, s), 7.07-7.35 (13H, m), 7.53 (1H, d, J=8.3 Hz), 7.65 (1H, d, J=7.9 Hz), 8.62 (1H, d, J=7.6 Hz), 9.56 (1H, br-s), 12.1 (1H, br-s).

### EXAMPLE 40

### Methyl 3-{2-[(2S)-2-[(2-quinolinylcarbonyl)-amino]-5-[benzyloxycarbonylamino]pentanoylamino]-phenyl}propanoate

The target compound was obtained in a similar manner to that of Example 27-3.

MS ((+)ESI) m/z: 605 (M+Na)⁺.

### EXAMPLE 41

### Sodium 3-{2-[(2S)-2-[(2-quinolinylcarbonyl)amino]-5-[benzyloxycarbonylamino]pentanoylamino]phenyl}-propanoate

To a suspension of methyl 3-{2-[(2S)-2-[(2-quinolinylcarbonyl)amino]-5-[benzyloxycarbonylamino]pentanoylamino]-phenyl}-propanoate (100 mg) obtained in Example 40 in EtOH (2.0 mL), was added 1N NaOH (0.343 mL). The mixture was refluxed for 10 minutes. The resulting solution was allowed to cool to room temperature, stirred for 16 hours, and concentrated in vacuo. The residual solid was dissolved in EtOH (2.0 mL) and the solution was stirred at room temperature for 2 hours. The resulting precipitates were collected by filtration, washed with EtOH, and dried under reduced pressure at 60° C. to give the target compound (79.3 mg) as a white solid.

MS ((−)ESI) m/z: 567 (M−Na)⁻.

¹H-NMR (200 MHz, DMSO-d₆): δ 1.55-1.58 (2H, m), 1.95-2.06 (2H, m), 2.27-2.30 (2H, m), 2.73-2.74 (2H, m), 3.12-3.14 (2H, m), 4.86-4.88 (1H, m), 4.98 (2H, s), 7.00-7.32 (8H, m), 7.70-7.90 (4H, m), 8.11 (1H, d, J=8.1 Hz), 8.21 (2H, d, J=8.5 Hz), 8.61 (1H, d, J=8.5 Hz), 9.01 (1H, d, J=8.4 Hz), 13.1 (1H, br-s).

### EXAMPLE 42-1

### Methyl 4-[2-({(2S)-5-{[benzyloxy)carbonyl]amino}-2-[(tert-butoxycarbonyl)amino]pentanoyl}amino)-ethyl]benzoate

To a suspension of (2S)-5-[[(benzyloxy)-carbonyl]amino]-2-[(tert-butoxycarbonyl)amino]-pentanoic acid (1.00 g) and methyl 4-(2-aminoethyl)benzoate hydrochloride (647 mg) in N, N-dimethylformamide (20 mL), were added HOBT (3.32 g), and WSCD (553 mg) at room temperature. The mixture was stirred for 2 hours.

The mixture was quenched by the addition of water (40 mL) and extracted with ethylacetate (40 mL×1). The extract was washed with water (40 mL×2), saturated aqueous sodium hydrogencarbonate (40 mL×1) and brine (40 mL×1), and then dried over magnesium sulfate.

Filtration followed by evaporation gave the target compound (1.45 g) as a pale yellow solid.

MS ((+) ESI) m/z: 550 (M+Na)⁺.

### EXAMPLE 42-2

### Methyl 4-{2-[((2S)-2-amino-5-{[(benzyloxy)-carbonyl]amino}pentanoyl)amino]ethyl}benzoate hydrochloride

Methyl 4-[2-[[(2S)-5-[[(benzyloxy)carbonyl]-amino]-2-[(tert-butoxycarbonyl)amino]pentanoyl]-amino]ethyl]benzoate (1.43 g) obtained in Example42-1 was suspended in 2.5N hydrogen chloride in methanol (14 mL). The mixture was stirred at room temperature for 16 hours. The solvent was removed by evaporation to give the target compound (1.27 g) as a yellow solid.

MS ((+)ESI) m/z: 450 (M+Na)⁺.

### EXAMPLE 42-3

### Methyl 4-{2-[( (2S)-2-[(1-benzofuran-2-ylcarbonyl)-amino]-5-{[(benzyloxy)carbonyl]amino}pentanoyl)-amino]ethyl}benzoate

The target compound was obtained in a similar manner to that of Example 27-3.

MS ((+)ESI) m/z: 594 (M+Na)⁺.

### EXAMPLE 43

### 4-{2-[((2S)-2-[(1-Benzofuran-2-ylcarbonyl)amino]-5-{[(benzyloxy)carbonyl]amino}pentanoyl)amino]-ethyl}benzoic acid

The target compound was obtained in a similar manner to that of Example 28.

MS ((−)ESI) m/z: 556 (M−H)⁻.

### EXAMPLE 44-1

### Methyl 6-({(2S)-5-{[(benzyloxy)carbonyl]amino}-2-[(tert-butoxycarbonyl)amino]pentanoyl}amino)-2-naphthoate

The target compound was obtained in a similar manner to that of Example 42-1.

MS ((+)ESI) m/z: 572 (M+Na)⁺.

### EXAMPLE 44-2

### Methyl 6-[((2S)-2-amino-5-{[(benzyloxy)carbonyl]-amino}pentanoyl)amino]-2-naphthoate hydrochloride

The target compound was obtained in a similar manner to that of Example 27-2.

MS ((+)ESI) m/z: 450 (M+H)⁺.

### EXAMPLE 44-3

### Methyl 6-[((2S)-2-[(1-benzofuran-2-ylcarbonyl)-amino]-5-{[(benzyloxy)carbonyl]amino}pentanoyl)-amino]-2-naphthoate

The target compound was obtained in a similar manner to that of Example 27-3.

MS ((+)ESI) m/z: 616 (M+Na)⁺.

### EXAMPLE 45

### 6-[((2S)-2-[(1-Benzofuran-2-ylcarbonyl)amino]-5-{[(benzyloxy)carbonyl]amino}pentanoyl)amino]-2-naphthoic acid

The target compound was obtained in a similar manner to that of Example 28.

MS ((−)ESI) m/z: 578 (M−H)⁻.

¹H-NMR (200 MHz, DMSO-d₆): δ 1.40-2.06 (4H, m), 2.96-3.48 (4H, m), 4.62-4.73 (1H, m), 5.01 (2H, s), 7.32-7.98 (14H, m), 8.09 (1H, d, J=8.5 Hz), 8.41 (1H, s), 8.54 (1H, s), 8.88 (1H, d, J=7.5 Hz), 10.5 (1H, br-s), 13.0 (1H, br-s).

### EXAMPLE 46-1

### Methyl 3′-({(2S)-5-{[(benzyloxy)carbonyl]amino}-2-[(tert-butoxycarbonyl)amino]pentanoyl}amino)-3-biphenylylcarboxylate

The target compound was obtained in a similar manner to that of Example 42-1.

MS ((+)ESI) m/z: 598 (M+Na)⁺.

### EXAMPLE 46-2

### Methyl 3′-[((2S)-2-amino-5-{[(benzyloxy)carbonyl]-amino}pentanoyl)amino]-3-biphenylylcarboxylate hydrochloride

The target compound was obtained in a similar manner to that of Example 27-2.

MS ((+) ESI) m/z: 476 (M+H)⁺.

### EXAMPLE 46-3

### Methyl 3′-[((2S)-2-[(1-benzofuran-2-ylcarbonyl)-amino]-5-{[(benzyloxy)carbonyl]amino}pentanoyl)-amino]-3-biphenylylcarboxylate

The target compound was obtained in a similar manner to that of Example 27-3.

MS ((+)ESI) m/z: 642 (M+Na)⁺.

### EXAMPLE 47

### 3′-[((2S)-2-[(1-Benzofuran-2-ylcarbonyl)amino]-5-{[(benzyloxy)carbonyl]amino}pentanoyl)amino]-3-biphenylylhenylcarboxylic acid

The target compound was obtained in a similar manner to that of Example 28.

MS ((−)ESI) m/z: 604 (M−H)⁻.

¹H-NMR (200 MHz, DMSO-d₆): δ 1.48-1.66 (2H, m), 1.83-1.96 (2H, m), 3.07-3.09 (2H, m), 4.58-4.69 (1H, m), 5.00 (2H, s), 7.26-8.01 (18H, m), 8.19 (1H, s), 8.82 (1H, d, J=7.5 Hz), 10.3 (1H, s), 13.1 (1H, br).

### EXAMPLE 48-1

### Methyl 3′-({(2S)-5-{[(benzyloxy)carbonyl]amino}-2-[(tert-butoxycarbonyl)amino]pentanoyl}amino)-4-biphenylylcarboxylate

The target compound was obtained in a similar manner to that of Example 42-1.

MS ((+)ESI) m/z: 598 (M+Na)⁺.

### EXAMPLE 48-2

### Methyl 3′-[((2S)-2-amino-5-{[(benzyloxy)carbonyl]-amino}pentanoyl)amino]-4-biphenylylcarboxylate hydrochloride

The target compound was obtained in a similar manner to that of Example 27-2.

MS ((+)ESI) m/z: 476 (M+H)⁺.

### EXAMPLE 48-3

### Methyl 3′-[((2S)-2-[(1-benzofuran-2-ylcarbonyl)-amino]-5-{[(benzyloxy)carbonyl]amino}pentanoyl)-amino]-4-biphenylylcarboxylate

The target compound was obtained in a similar manner to that of Example 27-3.

MS ((+)ESI) m/z: 642 (M+Na)⁺.

### EXAMPLE 49

### 3′-[((2S)-2-[(1-Benzofuran-2-ylcarbonyl)amino]-5-{[(benzyloxy)carbonyl]amino}pentanoyl)amino]-4-biphenylylcarboxylic acid

The target compound was obtained in a similar manner to that of Example 28.

MS ((−)ESI) m/z: 604 (M−H)⁻.

¹H-NMR (200 MHz, DMSO-d₆): δ 1.41-1.69 (2H, m), 1.80-1.97 (2H, m), 3.03-3.09 (2H, m), 4.58-4.69 (1H, m), 5.01 (2H, s), 7.29-7.53 (10H, m), 7.65-7.82 (6H, m), 8.02-8.06 (3H, m), 8.82 (1H, d, J=7.5 Hz),10.3 (1H, br-s), 13.0 (1H, br).

### EXAMPLE 50-1

### t-Butyl {2-[((2S)-2-(tert-butoxycarbonyl)amino-5-{[(benzyloxy)carbonyl]amino}pentanoyl)amino]-phenoxy}acetate

The target compound was obtained in a similar manner to that of Example 42-1.

MS ((+)ESI) m/z: 594 (M+Na)⁺.

### EXAMPLE 50-2

### Methyl {2-[((2S)-2-amino-5-{[(benzyloxy)carbonyl]-amino}pentanoyl)amino]phenoxy}acetate hydrochloride

The target compound was obtained in a similar manner to that of Example 42-2.

MS ((+)ESI) m/z: 430 (M+H)⁺.

### EXAMPLE 50-3

### Methyl {2-[((2S)-2-[(1-benzofuran-2-ylcarbonyl)-amino]-5-{[(benzyloxy)carbonyl]amino}pentanoyl)-amino]phenoxy}acetate

The target compound was obtained in a similar manner to that of Example 27-3.

MS ((+)ESI) m/z: 596 (M+Na)⁺.

### EXAMPLE 51

### Sodium {2-[((2S)-2-[(1-benzofuran-2-ylcarbonyl)-amino]-5-{[(benzyloxy)carbonyl]amino}pentanoyl)-amino]phenoxy}acetate

To a solution of methyl [2-[[(2S)-2-[(1-benzofuran-2-ylcarbonyl)amino]-5-[[(benzyloxy)carbonyl]amino]pentanoyl]amino]-phenoxy]acetate (197 mg) obtained in Example 50-3 in methanol (2.0 mL) and tetrahydrofuran (2.0 mL), was added 1N sodium hydroxide solution (0.343 mL). The mixture was stirred at room temperature for 20 hours. The solvent was removed by evaporation to give the target compound (220 mg) as a white solid.

MS ((−)ESI) m/z: 558 (M−Na)⁻.

¹H-NMR (200 MHz, DMSO-d₆): δ 1.56-1.97 (2H, m), 3.07-3.10 (2H, m), 4.20 (2H, s), 4.68-4.79 (1H, m), 5.00 (2H, s), 6.96-7.02 (3H, m), 7.33-7.80 (11H, m), 8.09-8.13 (1H, m), 8.89 (1H, d, J=8.5 Hz), 12.3 (1H, br-s).

### EXAMPLE 52-1

### tert-Butyl [3-({(2S)-5-{[(benzyloxy)carbonyl]-amino}-2-[(tert-butoxycarbonyl)amino]pentanoyl}-amino)phenoxy]acetate

The target compound was obtained in a similar manner to that of Example 42-1.

MS ((+)ESI) m/z: 594 (M+Na)⁺.

### EXAMPLE 52-2

### Methyl {3-[((2S)-2-amino-5-{[(benzyloxy)carbonyl]-amino}pentanoyl)amino]phenoxy}acetate hydrochloride

The target compound was obtained in a similar manner to that of Example 42-2.

MS ((+)ESI) m/z: 430 (M+H)⁺.

### EXAMPLE 52-3

### Methyl {3-[((2S)-2-[(1-benzofuran-2-ylcarbonyl)-amino]-5-{[(benzyloxy)carbonyl]amino}pentanoyl)-amino]phenoxy}acetate

The target compound was obtained in a similar manner to that of Example 27-3.

MS ((+)ESI) m/z: 594 (M+Na)⁺.

### EXAMPLE 53

### Sodium {3-[((2S)-2-[(1-benzofuran-2-ylcarbonyl)-amino]-5-{[(benzyloxy)carbonyl]amino}pentanoyl)-amino]phenoxy}acetate

The target compound was obtained in a similar manner to that of Example 51.

MS ((−)ESI) m/z: 558 (M−Na)⁺.

¹H-NMR (200 MHz, DMSO-d₆): δ 1.40-2.01 (4H, m), 3.03-3.06 (2H, m), 4.11 (2H, s), 4.57-4.60 (1H, m), 5.00 (2H, s), 6.52 (1H, d, J=8.0 Hz), 7.06-7.51 (11H, m), 7.67-7.80 (3H, m), 9.02 (1H, d, J=7.5 Hz), 10.3 (1H, br-s).

### EXAMPLE 54-1

### Methyl 3-[2-({(2S)-5-{[(benzyloxy)carbonyl]amino}-2-[(tert-butoxycarbonyl)amino]pentanoyl}amino)-ethyl]benzoate

The target compound was obtained in a similar manner to that of Example 42-1.

MS ((+)ESI) m/z: 550 (M+Na)⁺.

### EXAMPLE 54-2

### Methyl 3-{2-[((2S)-2-amino-5-{[(benzyloxy)-carbonyl]amino}pentanoyl)amino]ethyl}benzoate hydrochloride

The target compound was obtained in a similar manner to that of Example 27-2.

MS ((+)ESI) m/z: 428 (M+H)⁺.

### EXAMPLE 54-3

### Methyl 3-{2-[((2S)-2-[(1-benzofuran-2-ylcarbonyl)-amino]-5-{[(benzyloxy)carbonyl]amino}pentanoyl)-amino]ethyl}benzoate

The target compound was obtained in a similar manner to that of Example 27-3.

MS ((+)ESI) m/z: 594 (M+Na)⁺.

### EXAMPLE 55

### 3-{2-[((2S)-2-[(1-Benzofuran-2-ylcarbonyl)amino]-5-{[(benzyloxy)carbonyl]amino}pentanoyl)amino]-ethyl}benzoic acid

The target compound was obtained in a similar manner to that of Example 28.

MS ((−)ESI) m/z: 556 (M−H)⁻.

¹H-NMR (200 MHz, DMSO-d₆): δ 1.30-1.52 (2H, m), 1.60-1.82 (2H, m), 2.76-2.83 (2H, m), 2.95-3.01 (2H, m), 3.21-3.43 (2H, m), 4.08-4.45 (1H, m), 5.00 (2H, s), 7.24-7.80 (15H, m), 8.15 (1H, t, J=5.5 Hz), 8.52 (1H, d, J=8.0 Hz), 12.9 (1H, br).

### EXAMPLE 56-1

### Methyl 4′-({(2S)-5-{[(benzyloxy)carbonyl]amino}-2-[(tert-butoxycarbonyl)amino]pentanoyl}amino)-3-biphenylylcarboxylate

The target compound was obtained in a similar manner to that of Example 42-1.

MS ((+)ESI) m/z: 598 (M+Na)⁺.

### EXAMPLE 56-2

### Methyl 4′-[((2S)-2-amino-1-{[(benzyloxy)carbonyl]-amino}pentanoyl)amino]-3-biphenylylcarboxylate hydrochloride

The target compound was obtained in a similar manner to that of Example 27-2.

MS ((+)ESI) m/z: 498 (M+Na)⁺.

### EXAMPLE 56-3

### Methyl 4′-[((2S)-2-[(1-benzofuran-2-ylcarbonyl)-amino]-5-{[(benzyloxy)carbonyl]amino}pentanoyl)-amino]-3-biphenylylcarboxylate

The target compound was obtained in a similar manner to that of Example 27-3.

MS ((+)ESI) m/z: 642 (M+Na)⁺.

### EXAMPLE 57

### 4′-[((2S)-2-[(1-benzofuran-2-ylcarbonyl)amino]-5-{[(benzyloxy)carbonyl]amino}pentanoyl)amino]-3-biphenylylcarboxylic acid

The target compound was obtained in a similar manner to that of Example 28.

MS ((−)ESI) m/z: 604 (M−H)⁻.

¹H-NMR (200 MHz, DMSO-d₆): δ 1.48-1.69 (2H, m), 1.82-1.94 (2H, m), 3.03-3.13 (2H, m), 4.59-4.70 (1H, m), 5.01 (2H, s), 7.33-7.94 (18H, m), 8.18 (1H, s), 8.82 (1H, d, J=7.5 Hz), 10.3 (1H, br-s), 13.1 (1H, br).

### EXAMPLE 58-1

### Methyl 4-[2-({(2S)-5-amino-2-[(1-benzofuran-2-yl-carbonyl)amino]pentanoyl}amino)ethyl]benzoate

The target compound was obtained in a similar manner to that of Example 34-1.

MS ((+)ESI) m/z: 438 (M+H)⁺.

### EXAMPLE 58-2

### Methyl 4-[2-({(2S)-2-[(1-benzofuran-2-ylcarbonyl)-amino]-5-[(3-phenylpropanoyl)amino]pentanoyl}-amino)ethyl]benzoate

To a solution of methyl 4-[2-[[(2S)-5-amino-2-[(1-benzofuran-2-ylcarbonyl)-amino]pentanoyl]amino]ethyl]benzoate (100 mg) obtained in Example 58-1 and 3-phenylpropanoic acid (37.8 mg) in N,N-dimethylformamide (2.0 mL), were added HOBT (46.3 mg) and WSCD (87.6 mg). The mixture was stirred at room temperature for 16 hours.

The mixture was diluted with ethyl acetate (10 mL) washed successively with water (10 mL×2) and brine (10 mL), and dried over magnesium sulfate. Filtration followed by evaporation gave a crude product which was chromatographed on silica gel (SiO₂, 25 g, eluent: hexane/ethyl acetate=33/66 to 0/100) to give the target compound (78.2 mg) as a white solid.

MS ((+)ESI) m/z: 592 (M+Na)⁺.

### EXAMPLE 59

Sodium 4-[2-({(2S)-2-[(1-benzofuran-2-ylcarbonyl)-amino]-5-[(3-phenylpropanoyl)amino]pentanoyl}-amino)ethyl]benzoate

To a solution of methyl 4-(2-[[(2S)-2-[(1-benzofuran-2-ylcarbonyl)amino]-5-[(3-phenylpropanoyl)amino]pentanoyl]amino]ethyl]-benzoate (71.8 mg) obtained in Example 58-2 in methanol (1.0 mL) and tetrahydrofuran (1.0 mL), was added 1N sodium hydroxide (0.139 mL). The mixture was refluxed for 2 hours, at which time the reaction was incomplete. Additional 1N sodium hydroxide (0.025 mL) was added and the mixture was refluxed for 4 hours, at which time the starting material was still remained. Additional 1N sodium hydroxide (0.006 mL) was added and the mixture was refluxed for 2 hours, at which time the reaction was complete.

After cooling to room temperature, the solvent was removed by evaporation and the residual solid was washed a small amount of methanol, and dried under reduced pressure to give the target compound (23.1 mg) as a pale yellow crystalline solid.

MS ((−)ESI) m/z: 554 (M−Na)⁻.

¹H-NMR (200 MHz, DMSO-d₆): δ 1.25-1.36 (2H, m), 1.54-1.71 (2H, m), 2.32-2.40 (2H, m), 2.67-2.83 (4H, m), 2.93-3.03 (2H, m), 3.18-3.42 (2H, m), 4.35-4.45 (1H, m), 7.05-7.51 (9H, m), 7.64-7.80 (5H, m), 8.06 (1H, t, J=5.5 Hz), 8.18 (1H, t, J=5.5 Hz), 8.70 (1H, d, J=8.0 Hz).

### EXAMPLE 60

### Methyl 4-{2-[((2S)-2-[(1-benzofuran-2-ylcarbonyl)-amino]-5-{[(2R)-2-hydroxy-3-phenylpropanoyl]-amino}pentanoyl)amino]ethyl}benzoate

The target compound was obtained in a similar manner to that of Example 58-2.

MS ((+)ESI) m/z: 608 (M+Na)⁺.

### EXAMPLE 61

### Sodium 4-{2-[((2S)-2-[(1-benzofuran-2-ylcarbonyl)-amino]-5-{[(2R)-2-hydroxy-3-phenylpropanoyl]-amino}pentanoyl)amino]ethyl}benzoate

The target compound was obtained in a similar manner to that of Example 59.

MS ((−)ESI) m/z: 570 (M−Na)⁻.

¹H-NMR (200 MHz, DMSO-d₆): δ 1.19-1.40 (2H, m), 1.54-1.71 (2H, m), 2.66-2.82 (3H, m), 2.91-3.06 (3H, m), 3.17-3.46 (2H, m), 4.00-4.06 (1H, m), 4.35-4.45 (1H, m), 6.38 (1H, br), 7.07-7.51 (9H, m), 7.65-7.88 (6H, m), 8.22 (1H, t, J=5.0 Hz), 8.59 (1H, d, J=8.0 Hz).

### EXAMPLE 62

### Methyl 4-{2-[((2S)-2-[(1-benzofuran-2-ylcarbonyl)-amino]-5-{[(2S)-2-hydroxy-3-phenylpropanoyl3-amino}pentanoyl)amino]ethyl}benzoate

The target compound was obtained in a similar manner to that of Example 58-2.

MS ((+)ESI) m/z: 608 (M+Na)⁺.

### EXAMPLE 63

### Sodium 4-{2-[((2S)-2-[(1-benzofuran-2-ylcarbonyl)-amino]-5-{[(2S)-2-hydroxy-3-phenylpropanoyl]-amino}pentanoyl)amino]ethyl}benzoate

The target compound was obtained in a similar manner to that of Example 59.

MS ((−)ESI) m/z: 570 (M−Na)⁻.

¹H-NMR (200 MHz, DMSO-d₆): δ 1.23-1.42 (2H, m), 1.52-1.74 (2H, m), 2.66-2.81 (3H, m), 2.92-3.07 (2H, m), 3.21-3.43 (2H, m), 4.02-4.08 (1H, m), 4.35-4.46 (1H, m), 6.28 (1H, br), 7.08-7.50 (9H, m), 7.66-7.90 (6H, m), 8.27 (1H, t, J=5.0 Hz), 8.65 (1H, d, J=8.0 Hz).

### EXAMPLE 64

### Methyl 4-(2-{[(2S)-2-[(1-benzofuran-2-ylcarbonyl)-amino]-5-({[(2-chlorobenzyl)oxy]carbonyl}amino)-pentanoyl]amino}ethyl)benzoate

The target compound was obtained in a similar manner to that of Example 36-2.

MS ((+)ESI) m/z: 628 (M+Na)⁺.

### EXAMPLE 65

### 4-(2-{[(2S)-2-[(1-Benzofuran-2-ylcarbonyl)amino]-5-({[(2-chlorobenzyl)oxy]carbonyl}amino)-pentanoyl]amino}ethyl)benzoic acid

The target compound was obtained in a similar manner to that of Example 28.

MS ((−)ESI) m/z: 590 (M−H)⁻.

¹H-NMR (200 MHz, DMSO-d₆): δ 1.32-1.53 (2H, m), 1.60-1.81 (2H, m), 2.71-2.87 (2H, m), 2.93-3.07 (2H, m), 3.21-3.44 (2H, m), 4.34-4.45 (1H, m), 5.08 (2H, s), 7.30-7.86 (14H, m), 8.14 (1H, t, J=5.0 Hz), 8.52 (1H, d, J=8.0 Hz), 12.8 (1H, br).

### EXAMPLE 66

### Methyl 4-[2-({(2S)-2-[(1-benzofuran-2-ylcarbonyl)-amino]-5-[(isobutoxycarbonyl)amino]pentanoyl}-amino)ethyl]benzoate

The target compound was obtained in a similar manner to that of Example 36-2.

MS ((+) ESI) m/z: 560 (M+Na)⁺.

### EXAMPLE 67

### 4-[2-({(2S)-2-[(1-Benzofuran-2-ylcarbonyl)amino]-5-[(isobutoxycarbonyl)amino]pentanoyl}amino)-ethyl]benzoic acid

The target compound was obtained in a similar manner to that of Example 28.

MS ((−)ESI) m/z: 522 (M−H)⁻.

¹H-NMR (200 MHz, DMSO-d₆): δ 0.88 (6H, d, J=7.0 Hz), 1.28-1.87 (5H, m), 2.76-2.83 (2H, m), 2.92-3.01 (2H, m), 3.21-3.43 (2H, m), 3.70 (2H, d, J=7.0 Hz), 4.34-4.45 (1H, m), 7.08 (1H, t, J=5.5 Hz), 7.31-7.52 (4H, m), 7.62-7.86 (5H, m), 8.14 (1H, t, J=5.5 Hz), 8.52 (1H, d, J=8.0 Hz), 12.8 (1H, br).

### EXAMPLE 68-1

### Methyl 3-[2-({(2S)-2-[(tert-butoxycarbonyl)amino]-5-[(1H-imidazol-1-ylcarbonyl)amino]pentanoyl}-amino)phenyl]propanoate

To a solution of methyl 3-[2-[[(2S)-5-amino-2-[(tert-butoxycarbonyl)amino]pentanoyl]amino]phenyl]propanoate (6.36 g) in tetrahydrofuran (60 mL), was added 1,1′-carbonyldiimidazole (2.88 g). The mixture was stirred at room temperature for 3 hours.

The solvent was removed by evaporation and the residue was dissolved in ethyl acetate (60 mL). The solution was washed with brine (60 mL×1) and dried over magnesium sulfate. Filtration followed by evaporation gave a crude solid (8.33 g) which was chromatographed on silica gel (silica gel 500 g, eluent: chloroform/methanol=100/0 to 95/5) to give the target compound (7.88 g) as a pale yellow solid.

### EXAMPLE 68-2

### Methyl 3-{2-[((2S)-2-[(tert-butoxycarbonyl)amino]-5-{[(2-pyridinylmethoxy)carbonyl]amino}pentanoyl)-amino]phenyl}propanoate

To a solution of methyl 3-[2-[[(2S)-2-[(tert-butoxycarbonyl)amino]-5-[(1H-imidazol-1-ylcarbonyl)amino]pentanoyl]amino]phenyl]propanoate (500 mg) obtained in Example 68-1 in acetonitrile (5.0 mL), was added 2-pyridinemethanol (0.198 mL). The mixture was refluxed for 17 hours. After cooling to room temperature, the solvent was removed by evaporation and the residue was chromatographed on silica gel (eluent: chloroform/methanol=100/0 to 95/5) to give the target compound (226 mg) as a light brown solid.

### EXAMPLE 68-3

### Methyl 3-{2-[((2S)-2-[(1-benzothien-2-ylcarbonyl)-amino]-5-{[(2-pyridinylmethoxy)carbonyl]amino}-pentanoyl)amino]phenyl}propanoate

To a solution of methyl 3-[2-[[(2S)-2-[(tert-butoxycarbonyl)amino]-5-[[(2-pyridinylmethoxy)carbonyl]amino]pentanoyl]amino]-phenyl]propanoate (226 mg) obtained in Example 68-2 in ethyl acetate (1 mL), were added 4N hydrogen chloride in ethyl acetate (6 mL) and methanol (1 mL). The mixture was stirred at room temperature for 20 minutes. The solvent was removed by evaporation and the residue was dissolved in N,N-dimethylformamide (4 mL). To the solution, were added 1-benzothiophene-2-carboxylic acid (83.8 mg), 1-hydroxybenzotriazole (86.7 mg) and 1-(3-dimethylaminopropyl)-3-ethylcarbodiimide (0.195 mL). The mixture was stirred at room temperature for 3 hours.

The mixture was diluted with ethyl acetate (10 mL) washed with water (10 mL), saturated aqueous sodium hydrogencarbonate (10 mL), water (10 mL), and brine (10 mL), and dried over magnesium sulfate. Filtration followed by evaporation gave a solid which was suspended in chloroform (1 mL) and ethyl acetate (1 mL) After stirring for 1 hour, the precipitates were collected by filtration, washed with ethyl acetate and dried under reduced pressure to give the target compound (123 mg) as a pale orange solid.

MS ((+) ESI) m/z: 611 (M+Na)⁺.

### EXAMPLE 69

### Sodium 3-{2-[((2S)-2-[(1-benzothien-2-ylcarbonyl)-amino]-5-{[(2-pyridinylmethoxy)darbonyl]amino}-pentanoyl)amino]phenyl}propanoate

The target compound was obtained in a similar manner to that of Example 59.

MS ((−)ESI) m/z: 573 (M−Na)⁻.

¹H-NMR (200 MHz, DMSO-d₆): δ 1.48-1.73 (2H, m), 1.82-2.09 (2H, m), 2.21-2.37 (2H, m), 2.63-2.91 (2H, m), 3.03-3.23 (2H, m), 4.60-4.72 (1H, m), 5.06 (2H, s), 6.95-7.49 (8H, m), 7.74-8.04 (5H, m), 8.51 (1H, d, J=4.5 Hz), 8.62 (1H, s), 9.29 (1H, d, J=8.0 Hz), 12.5 (1H, br).

### EXAMPLE 70-1

### Methyl 3-{2-[((2S)-2-[(tert-butoxycarbonyl)amino]-5-{[(3-thienylmethoxy)carbonyl]amino}pentanoyl)-amino]phenyl}propanoate

The target compound was obtained in a similar manner to that of Example 68-2.

### EXAMPLE 70-2

### Methyl 3-{2-((2S)-2-[(1-benzothien-2-ylcarbonyl)-amino]-5-{[(3-thienylmethoxy)carbonyl]amino}-pentanoyl)amino]phenyl}propanoate

The target compound was obtained in a similar manner to that of Example 68-3.

MS ((+)ESI) m/z: 616 (M+Na)⁺.

### EXAMPLE 71

### 3-{2-[((2S)-2-[(1-Benzothien-2-ylcarbonyl)amino]-5-{[(3-thienylmethoxy)carbonyl]amino}pentanoyl)-amino]phenyl}propanoic acid

The target compound was obtained in a similar manner to that of Example 28.

MS ((−)ESI) m/z: 578 (M−H)⁻.

¹H-NMR (200 MHz, DMSO-d₆): δ 1.46-2.06 (4H, m), 2.43-2.57 (2H, m), 2.79-2.86 (2H, m), 3.03-3.13 (2H, m), 4.58-4.69 (1H, m) 44.99 (2H, s), 7.07-7.52 (10H, m), 7.90-8.08 (2H, m), 8.29 (1H, s), 7.73 (1H, d, J=7.5 Hz), 9.60 (1H, s), 12.2 (1H, br).

### EXAMPLE 72-1

### Methyl 3-{2-((2S)-2-(tert-butoxycarbonyl)amino]-5-{[(2-naphthylmethoxy)carbonyl]amino}pentanoyl)-amino]phenyl}propanoate

The target compound was obtained in a similar manner to that of Example 68-2.

### EXAMPLE 72-2

### Methyl 3-{2-((2S)-2-[(1-benzothien-2-ylcarbonyl)-amino]-5-{[(2-naphthylmethoxy)carbonyl]amino}-pentanoyl)amino]phenyl}propanoate

The target compound was obtained in a similar manner to that of Example 68-3.

MS ((+)ESI) m/z: 660 (M+Na)⁺.

### EXAMPLE 73

### 3-{2-[((2S)-2-[(1-Benzothien-2-ylcarbonyl)amino]-5-{[(2-naphthylmethoxy)carbonyl]amino}pentanoyl)-amino]phenyl}propanoic acid

The target compound was obtained in a similar manner to that of Example 28.

MS ((−)ESI) m/z: 622 (M−H)⁻.

¹H-NMR (200 MHz, DMSO-d₆): δ 1.44-2.05 (4H, m), 2.47-2.54 (2H, m), 2.75-2.86 (2H, m), 3.04-3.16 (2H, m), 4.61-4.71 (1H, m), 5.19 (2H, s), 7.00-7.54 (10H, m), 7.82-8.05 (6H, m), 8.30 (1H, s), 95 (1H, d, J=7.5 Hz), 9.61 (1H, s), 12-2 (1H, br).

### EXAMPLE 74-1

### Methyl 3-(2-{(2S)-2-(tert-butoxycarbonyl)amino]-5-({[(2-methylbenzyl)oxy]carbonyl}amino)-pentanoyl]amino}phenyl)propanoate

The target compound was obtained in a similar manner to that of Example 68-2.

### EXAMPLE 74-2

### Methyl 3-(2-{[(2S)-2-[(1-benzothien-2-ylcarbonyl)-amino]-5-({[(2-methylbenzyl)oxy]carbonyl}amino)-pentanoyl]amino}phenyl)propanoate

The target compound was obtained in a similar manner to that of Example 68-3.

MS ((+)ESI) m/z: 624 (M+Na)⁺.

### EXAMPLE 75

### 3-(2-{[(2S)-2-[(1-Benzothien-2-ylcarbonyl)amino]-5-({[(2-methylbenzyl)oxy]carbonyl}amino)-pentanoyl]amino}phenyl)propanoic acid

The target compound was obtained in a similar manner to that of Example 28.

MS ((−)ESI) m/z: 586 (M−H)⁻.

¹H-NMR(200 MHz, DMSO-d₆): δ 1.46-2.02 (4H, m), 2.27 (3H, s), 2.46-2.54 (2H, m), 2.74-2.86 (2H, m), 3.04-3.14 (2H, m), 4.60-4.70 (1H, m), 5.02 (2H, s), 7 10-7.51 (11H, m), 7.94-8.05 (2H, m), 8.30 (1H, s), 8.94 (1H, d, J=7.5 Hz), 9.60 (1H, s), 12.2 (1H, br).

### EXAMPLE 76-1

### Methyl 3-(2-{(2S)-2-[(tert-butoxycarbonyl) amino]-5-({[(3-methylbenzyl)oxy]carbonyl}amino)-pentanoyl]amino}phenyl)propanoate

The target compound was obtained in a similar manner to that of Example 68-2.

### EXAMPLE 76-2

### Methyl 3-(2-{[(2S)-2-[(1-benzothien-2-ylcarbonyl)-amino]-5-({[(3-methylbenzyl)oxy]carbonyl}amino)-pentanoyl]amino}phenyl)propanoate

The target compound was obtained in a similar manner to that of Example 68-3.

MS ((+)ESI) m/z: 624 (M+Na)⁺.

### EXAMPLE 77

### 3-(2-{[(2S)-2-[(1-Benzothien-2-ylcarbonyl)amino]-5-({[(3-methylbenzyl)oxy]carbonyl}amino)-pentanoyl]amino}phenyl)propanoic acid

The target compound was obtained in a similar manner to that of Example 28.

MS ((−)ESI) m/z: 586 (M−H)⁻.

¹H-NMR (200 MHz, DMSO-d₆): δ 1.47-2.00 (4H, m), 2.28 (3H, s), 2.46-2.54 (2H, m), 2.78-2.86 (2H, m), 3.05-3.14 (2H, m), 4.60-4.70 (1H, m), 4.97 (2H, s), 7.14-7.48 (11H, m), 7.94-8.05 (2H, m), 8.30 (1H, s), 8.94 (1H, d, J=7.5 Hz), 9.61 (1H, s), 12.2 (1H, br).

### EXAMPLE 78

### Methyl 3-[2-({(2S)-5-({[(2-chlorobenzyl)oxy]-carbonyl}amino)-2-[(2-quinolinylcarbonyl)amino]-pentanoyl}amino)phenyl]propanoate

The target compound was obtained in a similar manner to that of Example 27-3.

MS ((+)ESI) m/z: 639 (M+Na)⁺.

### EXAMPLE 79

### 3-[2-({(2S)-5-({[(2-Chlorobenzyl)oxy]carbonyl}-amino)-2-[(2-quinolinylcarbonyl)amino]pentanoyl}-amino)phenyl]propanoic acid

The target compound was obtained in a similar manner to that of Example 28.

MS ((−)ESI) m/z: 601 (M−H)⁻.

¹H-NMR (200 MHz, DMSO-d₆): δ 1.52-1.67 (2H, m), 1.86-2.07 (2H, m), 2.4 5-2.54 (2H, m), 2.779-2.87 (2H, m), 3.05-3.14 (2H, m), 4.80-4.90 (1H, m), 5.06 (2H, s), 7.15-7.57 (9H, m), 7.70-7.93 (2H, m), 8.09-8.22 (3H, m), 8.61 (1H, d, J=8.5 Hz), 8.91 (1H, d, J=8.5 Hz), 9.75 (1H, br-s), 12.2 (1H, br-s).

### EXAMPLE 80

### Benzyl{(4S)-4-[(1-benzofuran-2-ylcarbonyl)amino]-5-[(5-cyanopentyl)amino]-5-oxopentyl}carbamate

The target compound was obtained in a similar manner to that of Example 42-1.

### EXAMPLE 81

### Benzyl((4S)-4-[(1-benzofuran-2-ylcarbonyl)amino]-5-oxo-5-{[5-(2H-tetrazol-5-yl)pentyl]amino}-pentyl)carbamate

To a solution of benzyl [(4S)-4-[(1-benzofuran-2-ylcarbonyl)amino]-5-[(5-cyanopentyl)amino]-5-oxopentyl]carbamate (300 mg) obtained in Example 80 in 1-methyl-2-pyrrolidinone (6 mL), were added sodium azide (193 mg) and triethylamine hydrochloride (193 mg). The mixture was stirred at 140° C. for 20 hours.

After cooling to room temperature, the mixture was quenched by the addition of 1N hydrochloric acid (20 mL) and extracted with ethyl acetate (20 mL×1, 10 mL×1). The extracts were combined and washed with water (20 mL×2) and brine (20 mL×1), and dried over magnesium sulfate. Filtration followed by evaporation gave a crude product (280 mg) which was chromatographed on silica gel (eluent: chloroform/methanol=99/1 to95/5) to give the target compound (155 mg) as a yellow solid.

MS ((+)ESI) m/z: 570 (M+Na)⁺.

¹H-NMR (200 MHz, DMSO-d₆): δ 1.23-1.84 (10H, m), 2.83-3.13 (6H, m), 4.38-4.49 (1H, m), 5.01 (2H, s), 7.26-7.52 (8H, m), 7.64-7.81 (3H, m), 8.06 (1H, t, J=5.5 Hz), 8.52 (1H, d, J=8.0 Hz).

### EXAMPLE 82-1

### Ethyl 4-{2-[((2S)-2-amino-5-{[(benzyloxy)-carbonyl]amino}pentanoyl)amino]phenyl}butanoate hydrochloride

To a solution of ethyl 4-[2-(2s)-5-[[(benzyloxy)carbonyl]amino]-2-[(tert-butoxy-carbonyl)amino]pentanoyl]amino]phenyl]butanoate (518 mg) in 1,4-dioxane (1 mL), was added 4N hydrogen chloride in 1,4-dioxane (4 mL). The mixture was stirred at room temperature for 2 hours. The solvent was removed by evaporation to give the target compound (476 mg) as a pale yellow solid.

### EXAMPLE 82-2

### Ethyl 4-{2-[((2S)-5-{[(benzyloxy)carbonyl]amino}-2-{[(1-methyl-1H-indol-2-yl)carbonyl]amino}-pentanoyl)amino]phenyl}butanoate

The target compound was obtained in a similar manner to that of Example 27-3.

MS ((+)ESI) m/z: 635 (M+Na)⁺.

### EXAMPLE 83

### 4-12-[((2S)-5-{(Benzyloxy)carbonyl]amino}-2-{[(1-methyl-1H-indol-2-yl)carbonyl]amino}pentanoyl)-amino]phenyl}butanoic acid

The target compound was obtained in a similar manner to that of Example 28.

MS ((−)ESI) m/z: 583 (M−H)⁻.

¹H-NMR (200 MHz, DMSO-d₆): δ 1.53-1.95 (6H, m), 2.18-2.26 (2H, m), 2.55-2.63 (2H, m), 3.05-3.14 (2H, m), 3.99 (3H, s), 4.57-4.68 (1H, m), 5.02 (2H, s), 7.07-7.40 (13H, m), 7.53 (1H, d, J=8.0 Hz), 7.66 (1H, d, J=8.0 Hz), 8.61 (1H, d, J=7.5 Hz), 9.44 (1H, br-s), 12.1 (1H, br).

### EXAMPLE 84

### Ethyl 4-[2-({(2S)-5-{[(benzyloxy)carbonyl]amino}-2-[(2-quinolinylcarbonyl)amino]pentanoyl}amino)-phenyl]butanoate

The target compound was obtained in a similar manner to that of Example 27-3.

MS ((+)ESI) m/z: 633 (M+Na)⁺.

### EXAMPLE 85

### 4-[2-({(2S)-5-{[(Benzyloxy)carbonyl]amino}-2-[(2-quinolinylcarbonyl)amino]pentanoyl}amino)phenyl]-butanoic acid

The target compound was obtained in a similar manner to that of Example 28.

MS ((−)ESI) m/z: 581 (M−H)⁻.

¹H-NMR (200 MHz, DMSO-d₆): δ 1.51-1.78 (4H, m), 1.88-2.03 (2H, m), 2.17-2.24 (2H, m), 2.55-2.62 (2H, m), 4.80-4.90 (1H, m), 4.99 (2H, s), 7.15-7.42 (10H, m), 7.70-7.78 (1H, m), 7.85-7.93 (1H, m), 8.09-8.22 (2H, m), 8.61 (1H, d, J=8.0 Hz), 8.92 (1H, d, J=8.0 Hz), 9.65 (1H, br-s), 12.1 (1H, br).

### EXAMPLE 86-1

### Methyl 3-(2-{[(2S)-2-[(tert-butoxycarbonyl)amino]-5-({[(4-methylbenzyl)oxy]carbonyl}amino)-pentanoyl]amino}phenyl)propanoate

In a reaction vessel, was added a solution of methyl 3-[2-[[(2S)-2-[(tert-butoxycarbonyl)amino]-5-[(1H-imidazol-1-ylcarbonyl)amino]pentanoyl]amino]-phenyl]propanoate (500 mg) and (4-methylphenyl)-methanol (251 mg) in acetonitrile (5 mL). The vessel was placed in a microwave. The irradiation was adjusted to keep the temperature 140° C. and the reaction was performed for 2 hours. After cooling to room temperature, the solvent was removed by evaporation, and the residue was chromatographed on silica gel (eluent: hexane/ethyl acetate=2/1 to 1/1) to give the target compound (376 mg) as a white solid.

MS ((+)ESI) m/z: 564 (M+Na)⁺.

### EXAMPLE 86-2

### Methyl 3-(2-{[(2S)-2-amino-5-({[(4-methylbenzyl)-oxy]carbonyl}amino)pentanoyl]amino}phenyl)-propanoate hydrochloride

The target compound was obtained in a similar manner to that of Example 82-1.

### EXAMPLE 86-3

### Methyl 3-{2-[((2S)-5-({[(4-methylbenzyl)oxy]-carbonyl}amino)-2-{[(1-methyl-1H-indol-2-yl)-carbonyl]amino}pentanoyl)amino]phenyl}propanoate

The target compound was obtained in a similar manner to that of Example 27-3.

MS ((+)ESI) m/z: 621 (M+Na)⁺.

### EXAMPLE 87

### 3-{2-[((2S)-5-({[(4-Methylbenzyl)oxy]carbonyl}-amino)-2-{[(1-methyl-1H-indol-2-yl)carbonyl]-amino}pentanoyl)amino]phenyl}propanoic acid

The target compound was obtained in a similar manner to that of Example 28.

MS ((−)ESI) m/z: 583 (M−H)⁻.

¹H-NMR (200 MHz, DMSO-d₆): δ 1.52-1.69 (2H, m), 1.81-1.95 (2H, m), 2.27 (3H, s), 2.47-2.54 (2H, m), 2.79-2.86 (2H, m), 3.03-3.13 (2H, m), 3.98 (3H, s), 4.55-4.66 (1H, m), 4.96 (2H, s), 7.07-7.37 (12H, m), 7.53 (1H, d, J=8.0 Hz), 7.65 (1H, d, J=7.5 Hz), 8.62 (1H, d, J=7.5 Hz), 9.56 (1H, br-s), 12.1 (1H, br).

### EXAMPLE 88

### Methyl 3-[2-({(2S)-5-({[(4-methylbenzyl)oxy]-carbonyl}amino)-2-[(2-quinolinylcarbonyl)amino]-pentanoyl}amino)phenyl]propanoate

The target compound was obtained in a similar manner to that of Example 27-3.

MS ((+)ESI) m/z: 619 (M+Na)⁺.

### EXAMPLE 89

### 3-[2-({(2S)-5-({[(4-Methylbenzyl)oxy]carbonyl}-amino)-2-[(2-quinolinylcarbonyl)amino]pentanoyl}-amino)phenyl]propanoic acid

The target compound was obtained in a similar manner to that of Example 28.

MS ((−)ESI) m/z: 581 (M−H)⁻.

¹H-NMR (200 MHz, DMSO-d₆): δ 1.56-1.66 (2H, m), 1.85-2.06 (2H, m), 2.25 (3H, s), 2.45-2.51 (2H, m), 2.80-2.87 (2H, m), 3.04-3.13 (2H, m), 4.81-4.87 (1H, m), 4.94 (2H, s), 7.10-7.40 (9H, m), 7.71-7.93 (2H, m), 8.09-8.23 (3H, m), 8.61 (1H, d, J=8.5 Hz), 8.92 (1H, d, J=8.5 Hz), 9.76 (1H, br-s), 12.2 (1H, br).

### EXAMPLE 90-1

### Methyl 3-{2-[((2S)-2-[(tert-butoxycarbonyl)amino]-5-{[(3-furylmethoxy)carbonyl]amino}pentanoyl)-amino]phenyl}propanoate

The target compound was obtained in a similar manner to that of Example 86-1.

MS ((+)ESI) m/z: 540 (M+Na)⁺.

### EXAMPLE 90-2

### Methyl 3-{2-[((2S)-2-amino-5-{[(3-furylmethoxy)-carbonyl]amino}pentanoyl)amino]phenyl}propanoate hydrochloride

The target compound was obtained in a similar manner to that of Example 82-1.

### EXAMPLE 90-3

### Methyl 3-{2-[((2S)-5-{[(3-furylmethoxy)carbonyl]-amino}-2-{[(1-methyl-1H-indol-2-yl)carbonyl]-amino}pentanoyl)amino]phenyl}propanoate

The target compound was obtained in a similar manner to that of Example 27-3.

MS ((+)ESI) m/z: 597 (M+Na)⁺.

### EXAMPLE 91

### 3-{2-[((2S)-5-{[(3-Furylmethoxy)carbonyl]amino}-2-{[(1-methyl-1H-indol-2-yl)carbonyl]amino}-pentanoyl)amino]phenyl}propanoic acid

The target compound was obtained in a similar manner to that of Example 28.

MS ((−)ESI) m/z: 559 (M−H)⁻.

¹H-NMR (200 MHz, DMSO-d₆): δ 1.51-1.69 (2H, m), 1.81-1.94 (2H, m), 2.46-2.54 (2H, m), 2.79-2.86 (2H, m), 3.03-3.12 (2H, m), 3.98 (3H, s), 4.55-4.65 (1H, m), 4.86 (2H, s ), 6.48 (1H, d, J=1.5 Hz), 7.07-7.37 (8H, m), 7.51-7.68 (4H, m), 8.62 (1H, d, J=7.5 Hz), 9.55 (1H, br-s), 12.1 (1H, br).

### EXAMPLE 92

### Methyl 3-[2-({(2S)-5-{[(3-furylmethoxy)carbonyl]-amino}-2-[(2-quinolinylcarbonyl) amino]pentanoyl}-amino)phenyl]propanoate

The target compound was obtained in a similar manner to that of Example 27-3.

MS ((+)ESI) m/z: 595 (M+Na)⁺.

### EXAMPLE 93

### 3-[2-({(2S)-5-{(3-Furylmethoxy)carbonyl]amino}-2-[(2-quinolinylcarbonyl)amino]pentanoyl}amino)-phenyl]propanoic acid

The target compound was obtained in a similar manner to that of Example 28.

MS ((−)ESI) m/z: 557 (M−H)⁻.

¹H-NMR (200 MHz, DMSO-d₆): δ 1.51-1.65 (2H, m), 1.85-2.06 (2H, m), 2.45-2.53 (2H, m), 2.79-2.87 (2H, m), 3.03-3.11 (2H, m), 4.79-4.90 (3H, m), 6.46 (1H, s), 7.11-7.39 (5H, m), 7.59-7.90 (4H, m), 8.09-8.22 (3H, m), 8.61 (H, d, J=8.5 Hz), 8.91 (1H, d, J=8.0 Hz), 9.75 (1H, br-s), 12.1 (1H, br).

### EXAMPLE 94-1

### Methyl 3-{2-[((2S)-2-[(tert-butoxycarbonyl)amino]-5-{[(3-pyridinylmethoxy)carbonyl]amino}pentanoyl)-amino]phenyl}propanoate

The target compound was obtained in a similar manner to that of Example 86-1.

MS ((+)ESI) m/z: 5 5 1 (M+Na)⁺.

### EXAMPLE 94-2

### Methyl 3-{2-[((2S)-2-amino-5-{[(3-pyridinyl-methoxy)carbonyl]amino}pentanoyl)amino]phenyl}-propanoate dihydrochloride

The target compound was obtained in a similar manner to that of Example 82-1.

### EXAMPLE 94-3

### Methyl 3-{2-[((2S)-2-{[(1-methyl-1H-indol-2-yl)-carbonyl]amino}-5-{[(3-pyridinylmethoxy)carbonyl]-amino}pentanoyl)amino]phenyl}propanoate

The target compound was obtained in a similar manner to that of Example 27-3.

MS ((+) ESI) m/z: 608 (M+Na)⁺.

### EXAMPLE 95

### Sodium 3-{2-[((2S)-2-{[(1-methyl-1H-indol-2-yl)-carbonyl]amino}-5-{[(3-pyridinylmethoxy)carbonyl]-amino}pentanoyl)amino]phenyl}propanoate

The target compound was obtained in a similar manner to that of Example 59.

MS ((−)ESI) m/z: 570 (M−Na)⁻.

¹H-NMR (200 MHz, DMSO-d₆): δ 1.50-1.68 (2H, m), 1.81-2.04 (2H, m), 2.25-2.30 (2H, m), 2.73-2.78 (2H, m), 3.07-3.16 (2H, m), 3.99 (3H, s), 4.61-4.72 (1H, m), 5.04 (2H, s), 6.97-7.15 (4H, m), 7.23-7.65 (6H, m), 7.75-7.85 (3H, m), 8.50 (1H, dd, J=1.5, 4.5 Hz), 8.57 (1H, d, J=2.0 Hz), 8.74 (1H, d, J=8.5 Hz).

### EXAMPLE 96

### Methyl 3-[2-({(2S)-5-{[(3-pyridinylmethoxy)-carbonyl]amino}-2-[(2-quinolinylcarbonyl)amino]-pentanoyl}amino)phenyl]propanoate

The target compound was obtained in a similar manner to that of Example 27-3.

MS ((+)ESI) m/z: 606 (M+Na)⁺.

### EXAMPLE 97

### Sodium 3-[2-({(2S)-5-{[(3-pyridinylmethoxy)-carbonyl]amino}-2-[(2-quinolinylcarbonyl)amino]-pentanoyl}amino)phenyl]propanoate

The target compound was obtained in a similar manner to that of Example 59.

MS ((−)ESI) m/z: 568 (M−Na)⁻.

¹H-NMR (200 MHz, DMSO-d₆): δ 1.46-1.68 (2H, m), 1.85-2.13 (2H, m),2.28-2.31 (2H, m), 2.64-2.86 (2H, m), 3.10-3.18 (2H, m), 4.82-4.92 (1H, m), 5.03 (2H, s), 6.97-7.18 (3H, m), 7.33-7.39 (1H, m), 7.70-7.94 (5H, m), 8.11 (1H, d, J=8.0 Hz), 8.21 (1H, d, J=8.5 Hz), 8.48-8.63 (3H, m) 9.00 (1H, d, J=8.5 Hz), 13.0 (1H, br-s).

### EXAMPLE 98-1

### Methyl 3-{2-[((2S)-2-[(tert-butoxycarbonyl)amino]-5-{[(4-pyridinylmethoxy)carbonyl]amino}pentanoyl)-amino]phenyl}propanoate

The target compound was obtained in a similar manner to that of Example 86-1.

MS ((+)ESI) m/z: 551 (M+Na)⁺.

### EXAMPLE 98-2

### Methyl 3-{2-[((2S)-2-amino-5-{[(4-pyridinyl-methoxy)carbonyl]amino}pentanoyl)amino]phenyl}-propanoate dihydrochloride

The target compound was obtained in a similar manner to that of Example 82-1.

### EXAMPLE 98-3

### Methyl 3-[2-({(2S)-5-{[(4-pyridinylmethoxy)-carbonyl]amino}-2-[(2-quinolinylcarbonyl)amino]-pentanoyl}amino)phenyl]propanoate

The target compound was obtained in a similar manner to that of Example 27-3.

MS ((+)ESI) m/z: 606 (M+Na)⁺.

### EXAMPLE 99

### Sodium 3-[2-({(2S)-5-{[(4-pyridinylmethoxy)-carbonyl]amino}-2-[(2-quinolinylcarbonyl)amino]-pentanoyl}amino)phenyl]propanoate

The target compound was obtained in a similar manner to that of Example 59.

MS ((−)ESI) m/z: 568 (M−Na)⁻.

¹H-NMR (200 MHz, DMSO-d₆): δ 1.45-1.73 (2H, m), 1.86-2.18 (2H, m), 2.26-2.36 (2H, m), 2.70-2.84 (2H, m), 3.12-3.21 (2H, m), 4.84-4.95 (1H, m) 5.04 (2H, s), 7.01-7.18 (3H, m), 7.31 (2H, d, J=5.5 Hz), 7.70-8.13 (5H, m), 8.22 (2H, d, J=8.5 Hz), 8.51 (2H, d, J=6.0 Hz), 8.61 (1H, d, J=8.5 Hz), 9.01 (1H, d, J=8.5 Hz), 13.0 (1H, br-s).

### EXAMPLE 100-1

### Methyl 3-(2-{[(2S)-2-[(tert-butoxycarbonyl)-amino]-5-({[(3-chlorobenzyl)oxy]carbonyl}amino)-pentanoyl]amino}phenyl)propanoate

The target compound was obtained in a similar manner to that of Example 86-1.

MS ((+)ESI) m/z: 584 (M+Na)⁺.

### EXAMPLE 100-2

### Methyl 3-(2-{[(2S)-2-amino-5-({[(3-chlorobenzyl)-oxy]carbonyl}amino)pentanoyl]amino}phenyl)-propanoate hydrochloride

The target compound was obtained in a similar manner to that of Example 82-1.

### EXAMPLE 100-3

### Methyl 3-{2-[((2S)-5-({[(3-chlorobenzyl)oxy]-carbonyl}amino)-2-{[(1-methyl-1H-indol-2-yl)-carbonyl]amino}pentanoyl)amino]phenyl}propanoate

The target compound was obtained in a similar manner to that of Example 27-3.

MS ((+)ESI) m/z: 641 (M+Na)⁺.

### EXAMPLE 101

### 3-{2-[((2S)-5-({[(3-Chlorobenzyl)oxy]carbonyl}-amino)-2-{[(1-methyl-1H-indol-2-yl)carbonyl]-amino}pentanoyl)amino]phenyl}propanoic acid

The target compound was obtained in a similar manner to that of Example 28.

MS ((−)ESI) m/z: 603 (M−H)⁻.

¹H-NMR (200 MHz, DMSO-d₆): δ 1.50-1.70 (2H, m), 1.83-1.96 (2H, m), 2.47-2.55 (2H, m), 2.79-2.87 (2H, m), 3.05-3.14 (2H, m), 4.01 (3H, s), 4.56-4.66 (1H, m), 5.02 (2H, s ), 7.0 7-7.41 (12H, m), 7.53 (1H, d, J=8.0 Hz), 7.65 (1H, d, J=8.0 Hz), 8.63 (1H, d, J=8.0 Hz), 9.55 (1H, br-s), 12.1 (1H, br).

### EXAMPLE 102

### Methyl 3-[2-({(2S)-5-({[(3-chlorobenzyl)oxy]-carbonyl}amino)-2-[(2-quinolinylcarbonyl)amino]-pentanoyl}amino)phenyl]propanoate

The target compound was obtained in a similar manner to that of Example 27-3.

MS ((+)ESI) m/z: 639 (M+Na)⁺.

### EXAMPLE 103

### 3-[2-({(2S)-5-({[(3-Chlorobenzyl)oxy]carbonyl}-amino)-2-[(2-quinolinylcarbonyl)amino]pentanoyl}-amino)phenyl]propanoic acid

The target compound was obtained in a similar manner to that of Example 28.

MS ((−)ESI) m/z: 601 (M−H)⁻.

¹H-NMR (200 MHz, DMSO-d₆): δ 1.53-1.68 (2H, m), 1.87-2.08 (2H, m), 2.46-2.55 (2H, m), 2.81-2.88 (2H, m), 3.05-3.14 (2H, m), 4.82-4.92 (1H, m), 5.00 (2H, s), 7.13-7.38 (9H, m), 7.74 (1H, t, J=7.0 Hz), 7.89 (1H, t, J=7.0 Hz), 8.09-8.22 (3H, m), 8.61 (1H, d, J=8.5 Hz), 8.92 (1H, d, J=8.0 Hz), 9.76 (1H, br-s), 12.2 (1H, br).

### EXAMPLE 104-1

### Methyl 3-(2-{[(2S)-2-[(tert-butoxycarbonyl)-amino]-5-({[(4-chlorobenzyl)oxy]carbonyl}amino)-pentanoyl]amino}phenyl)propanoate

The target compound was obtained in a similar manner to that of Example 86-1.

MS ((+)ESI) m/z: 584 (M+Na)⁺.

### EXAMPLE 104-2

### Methyl 3-(2-{[(2S)-2-amino-5-({[(4-chlorobenzyl)-oxy]carbonyl}amino)pentanoyl]amino}phenyl)-propanoate hydrochloride

The target compound was obtained in a similar manner to that of Example 82-1.

### EXAMPLE 104-3

### Methyl 3-{2-[((2S)-5-({[(4-chlorobenzyl)oxy]-carbonyl}amino)-2-{[(1-methyl-1H-indol-2-yl)-carbonyl]amino}pentanoyl)amino]phenyl}propanoate

The target compound was obtained in a similar manner to that of Example 27-3.

MS ((+)ESI) m/z: 641 (M+Na)⁺.

### EXAMPLE 105

### 3-{2-[((2S)-5-({(4-Chlorobenzyl) oxy]carbonyl}-amino)-2-{(1-methyl-1H-indol-2-yl) carbonyl]-amino}pentanoyl)amino]phenyl}propanoic acid

The target compound was obtained in a similar manner to that of Example 28.

MS ((−)ESI) m/z: 603 (M−H)⁻.

¹H-NMR (200 MHz, DMSO-d₆): δ 1.53-1.70 (2H, m), 1.82-1.96 (2H, m),2.47-2.55 (2H, m), 2.79-2.87 (2H, m), 3.04-3.14 (2H, m), 3.98 (3H, s), 4.56-4.67 (1H, m), 5.01 (2H, s), 7.07-7.44 (12H, m), 7.53 (1H, d, J=8.5 Hz), 7.66 (1H, d, J=7.5 Hz), 8.62 (1H, d, J=7.5 Hz), 9.55 (1H, br-s), 12.1 (1H, br).

### EXAMPLE 106

### Methyl 3-[2-({(2S)-5-({[(4-chlorobenzyl)oxy]-carbonyl}amino)-2-[(2-quinolinylcarbonyl)amino]-pentanoyl}amino)phenyl]propanoate

The target compound was obtained in a similar manner to that of Example 27-3.

MS ((+)ESI) m/z: 639 (M+Na)⁺.

### EXAMPLE 107

### 3-[2-({(2S)-5-({[(4-Chlorobenzyl)oxy]carbonyl}-amino)-2-[(2-quinolinylcarbonyl)amino]pentanoyl}-amino)phenyl]propanoic acid

The target compound was obtained in a similar manner to that of Example 28.

MS ((−)ESI) m/z: 601 (M−H)⁻.

¹H-NMR (200 MHz, DMSO-d₆): δ 1.52-1.67 (2H, m), 1.86-2.07 (2H, m), 2.46-2.54 (2H, m), 2.80-2.88 (2H, m), 3.04-3.14 (2H, m), 4.81-4.91 (1H, m), 4.9 (2H, s), 7.16-7.40 (9H, m), 7.70-7.93 (2H, m), 8.09-8.23 (3H, m), 8.61 (1H, d, J=8.5 Hz) 8.92 (1H, d, J=8.0 Hz), 9.75 (1H, br-s), 12.2 (1H, br).

### EXAMPLE 108-1

### Methyl 3-(2-{[(2S)-2-[(tert-butoxycarbonyl)-amino]-5-({[(2-methylbenzyl)oxy]carbonyl}amino)-pentanoyl]amino}phenyl)propanoate

The target compound was obtained in a similar manner to that of Example 86-1.

### EXAMPLE 108-2

### Methyl 3-(2-{[(2S)-2-amino-5-({[(2-methylbenzyl)-oxy]carbonyl}amino)pentanoyl]amino}phenyl)-propanoate hydrochloride

The target compound was obtained in a similar manner to that of Example 82-1.

### EXAMPLE 108-3

### Methyl 3-{2-[((2S)-5-({[(2-methylbenzyl)oxy]-carbonyl}amino)-2-{[(1-methyl-1H-indol-2-yl)-carbonyl]amino}pentanoyl)amino]phenyl}propanoate

The target compound was obtained in a similar manner to that of Example 27-3.

MS ((+)ESI) m/z: 621 (M+Na)⁺.

### EXAMPLE 109

### 3-12-[((2S)-5-({[(2-Methylbenzyl)oxy]carbonyl}-amino)-2-{[(1-methyl-1H-indol-2-yl)carbonyl]-amino}pentanoyl)amino]phenyl}propanoic acid

The target compound was obtained in a similar manner to that of Example 28.

MS ((−)ESI) m/z: 583 (M−H)⁻.

¹H-NMR (200 MHz, DMSO-d₆): δ 1.52-1.69 (2H, m), 1.81-1.95 (2H, m), 2.27 (3H, s), 2.46-2.54 (2H, m), 2.79-2.86 (2H, m), 3.04-3.13 (2H, m), 3.98 (3H, s), 4.55-4.66 (1H, m), 5.01 (2H, s), 7.07-7.36 (12H, m), 7.53 (1H, d, J=8.0 Hz), 7.65 (1H, d, J=8.0 Hz), 8.62 (1H, d, J=7.5 Hz), 9.56 (1H, br-s), 12.1 (1H, br).

### EXAMPLE 110

### Methyl 3-[2-({(2S)-5-({[(2-methylbenzyl)oxy]-carbonyl}amino)-2-[(2-quinolinylcarbonyl)amino]-pentanoyl}amino)phenyl]propanoate

The target compound was obtained in a similar manner to that of Example 27-3.

MS ((+) ESI) m/z: 619 (M+Na)⁺.

### EXAMPLE 111

### 3-[2-({(2S)-5-({[(2-Methylbenzyl)oxy]carbonyl}-amino)-2-[(2-quinolinylcarbonyl)amino]pentanoyl}-amino)phenyl]propanoic acid

The target compound was obtained in a similar manner to that of Example 28.

MS ((−)ESI) m/z: 581 (M−H)⁻.

¹H-NMR (200 MHz, DMSO-d₆): δ 1.51-1.66 (2H, m), 1.86-2.07 (2H, m), 2.25 (3H, s), 2.45-2.53 (2H, m), 2.80-2.87 (2H , m), 3.04-3.13 (2H, m), 4.80-4.91 (1H, m), 4.99 (2H, s), 7.12-7.40 (9H, m), 7.70-7.93 (2H, m), 8.09-8.22 (3H, m), 8.61 (1H, d, J=8.5 Hz), 8.91 (1H, d, J=8.5 Hz), 9.76 (1H, br-s), 12.2 (1H, br).

### EXAMPLE 112-1

### Methyl 3-(2-{[(2S)-2-[(tert-butoxycarbonyl)-amino]-5-({[(3-methylbenzyl)oxy]carbonyl}amino)-pentanoyl]amino}phenyl)propanoate

The target compound was obtained in a similar manner to that of Example 86-1.

### EXAMPLE 112-2

### Methyl 3-(2-{(2S)-2-amino-5-({[(3-methylbenzyl)-oxy]carbonyl}amino)pentanoyl]amino}phenyl)-propanoate hydrochloride

The target compound was obtained in a similar manner to that of Example 82-1.

### EXAMPLE 112-3

### Methyl 3-{2-[((2S)-5-({[(3-methylbenzyl)oxy]-carbonyl}amino)-2-{[(1-methyl-1H-indol-2-yl)-carbonyl]amino}pentanoyl)amino]phenyl}propanoate

The target compound was obtained in a similar manner to that of Example 27-3.

MS ((+)ESI) m/z: 621 (M+Na)⁺.

### EXAMPLE 113

### 3-{2-[((2S)-5-({[(3-Methylbenzyl)oxy]carbonyl}-amino)-2-{[(1-methyl-1H-indol-2-yl)carbonyl]-amino}pentanoyl)amino]phenyl}propanoic acid

The target compound was obtained in a similar manner to that of Example 28.

MS ((−)ESI) m/z: 583 (M−H)⁻.

¹H-NMR (200 MHz, DMSO-d₆): δ 1.53-1.70 (2H, m), 1.82-1.96 (2H, m), 2.28 (3H, s), 2.47-2.54 (2H, m), 2.79-2.87 (2H, m), 3.05-3.14 (2H, m), 3.98 (3H, s), 4.56-4.66 (1H, m), 4.97 (2H, s), 7.07-7.36 (12H, m), 7.53 (1H, d, J=8.5 Hz), 7.65 (1H, d, J=8.5 Hz), 8.63 (1H, d, J=7.5 Hz), 9.56 (1H, br-s), 12.2 (1H, br).

### EXAMPLE 114

### Methyl 3-[2-({(2S)-5-({[(3-methylbenzyl)oxy]-carbonyl}amino)-2-[(2-quinolinylcarbonyl)amino]-pentanoyl}amino)phenyl]propanoate

The target compound was obtained in a similar manner to that of Example 27-3.

MS ((+)ESI) m/z: 619 (M+Na)⁺.

### EXAMPLE 115

### 3-[2-({(2S)-5-({[(3-Methylbenzyl)oxy]carbonyl}-amino)-2-[(2-quinolinylcarbonyl)amino]pentanoyl}-amino)phenyl]propanoic acid

The target compound was obtained in a similar manner to that of Example 28.

MS ((−)ESI) m/z: 581 (M−H)⁻.

¹H-NMR (200 MHz, DMSO-d₆): δ 1.54-1.67 (2H, m), 1.90-2.04 (2H, m), 2.27 (3H, s), 2.46-2.54 (2H, m), 2.81-2.88 (2H, m), 3.05-3.14 (2H, m), 4.81-4.92 (1H, m), 4.95 (2H, s), 7.12-7.39 (9H, m), 7.71-7.93 (2H, m), 8.09-8.23 (3H, m), 8.61 (1H, d, J=8.5 Hz), 8.92 (1H, d, J=8.0 Hz), 9.76 (1H, br-s), 12.2 (1H, br).

### EXAMPLE 116-1

### Methyl 3-[({(2S)-5-{[(benzyloxy)carbonyl]amino}-2-[(tert-butoxycarbonyl)amino]pentanoyl}amino)-methyl]benzoate

The target compound was obtained in a similar manner to that of Example 42-1.

MS ((+)ESI) m/z: 536 (M+Na)⁺.

### EXAMPLE 116-2

### Methyl 3-{[((2S)-2-amino-5-{(benzyloxy)carbonyl]-amino}pentanoyl)amino]methyl}benzoate hydrochloride

The target compound was obtained in a similar manner to that of Example 82-1.

MS ((+)ESI) m/z: 436 (M+Na)⁺.

### EXAMPLE 116-3

### Methyl 3-{[((2S)-5-{[(benzyloxy)carbonyl]amino}-2-{[(1-methyl-1H-indol-2-yl)carbonyl]amino}-pentanoyl)amino]methyl}benzoate

The target compound was obtained in a similar manner to that of Example 27-3.

MS ((+)ESI) m/z: 593 (M+Na)⁺.

### EXAMPLE 117

### 3-{[((2S)-5-{[(Benzyloxy)carbonyl]amino}-2-{[(1-methyl-1H-indol-2-yl)carbonyl]amino}pentanoyl)-amino]methyl}benzoic acid

The target compound was obtained in a similar manner to that of Example 28.

MS ((−)ESI) m/z: 555 (M−H)⁻.

¹H-NMR (200 MHz, DMSO-d₆): δ 1.44-1.87 (4H, m), 3.00-3.09 (2H, m), 3.97 (3H, s), 4.37 (2H, d, J=6.0 Hz), 4.42-4.50 (1H, m), 5.00 (2H, s), 7.08-7.89 (15H, m), 8.50-8.60 (2H, m), 12.9 (1H, br).

### EXAMPLE 118

### Methyl 3-[({(2S)-5-{[(benzyloxy)carbonyl]amino}-2-[(2-quinolinylcarbonyl)amino]pentanoyl}amino)-methyl]benzoate

The target compound was obtained in a similar manner to that of Example 27-3.

MS ((+)ESI) m/z: 591 (M+Na)⁺.

### EXAMPLE 119

### 3-[({(2S)-5-{[(Benzyloxy)carbonyl]amino}-2-[(2-quinolinylcarbonyl)amino]pentanoyl}amino)methyl]-benzoic acid

The target compound was obtained in a similar manner to that of Example 28.

MS ((−)ESI) m/z: 553 (M−H)⁻.

¹H-NMR (200 MHz, DMSO-d₆): δ 1.45-1.59 (2H, m), 1.80-1.95 (2H, m), 3.01-3.11 (2H, m), 4.42 (2H, d, J=5.5 Hz), 4.62-4.72 (1H, m), 5.00 (2H, s), 7.2 5-7.57 (7H, m), 7.71-7.93 (4H, m), 8.08-8.22 (3H, m), 8.60 (1H, d, J=8.5 Hz), 8.80-8.89 (2H, m), 12.9 (1H, br).

### EXAMPLE 120-1

### Methyl{3-[({(2S)-5-{[(benzyloxy)carbonyl]amino}-2-[(tert-butoxycarbonyl)amino]pentanoyl}amino)-methyl]phenyl}acetate

The target compound was obtained in a similar manner to that of Example 42-1.

MS ((+) ESI) m/z: 550 (M+Na)⁺.

### EXAMPLE 120-2

### Methyl(3-{[((2S)-2-amino-5-{[(benzyloxy)-carbonyl]amino}pentanoyl)amino]methyl}phenyl)-acetate hydrochloride

The target compound was obtained in a similar manner to that of Example 82-1.

MS ((+)ESI) m/z: 428 (M+H)⁺.

### EXAMPLE 120-3

### Methyl(3-{[((2S)-5-{[(benzyloxy)carbonyl]amino}-2-{[(1-methyl-1H-indol-2-yl)carbonyl]amino)-pentanoyl)amino]methyl}phenyl)acetate

The target compound was obtained in a similar manner to that of Example 27-3.

MS ((+)ESI) m/z: 607 (M+Na)⁺.

### EXAMPLE 121

### (3-{[((2S)-5-{(Benzyloxy)carbonyl]amino}-2-{[(1-methyl-1H-indol-2-yl)carbonyl]amino}pentanoyl)-amino]methyl}phenyl)acetic acid

The target compound was obtained in a similar manner to that of Example 28.

MS ((−)ESI) m/z: 569 (M−H)⁻.

¹H-NMR (200 MHz, DMSO-d₆): δ 1.40-1.88 (4H, m), 3.00-3.09 (2H, m), 3.53 (2H, s), 3.97 (3H, s), 4.30 (2H, d, J=6.0 Hz), 4.39-4.50 (1H, m), 5.00 (2H, s), 7.06-7.66 (15H, m), 8.50 (2H, d, J=5.0 Hz), 12.3 (1H, br).

### EXAMPLE 122

### Methyl{3-({(2S)-5-{[(benzyloxy)carbonyl]amino}-2-[(2-quinolinylcarbonyl)amino]pentanoyl}amino)-methyl]phenyl}acetate

The target compound was obtained in a similar manner to that of Example 27-3.

MS ((+)ESI) m/z: 605 (M+Na)⁺.

### EXAMPLE 123

### {3-[({(2S)-5-{(Benzyloxy)carbonyl]amino}-2-[(2-quinolinylcarbonyl)amino]pentanoyl}amino)methyl]-phenyl}acetic acid

The target compound was obtained in a similar manner to that of Example 28.

MS ((−)ESI) m/z: 567 (M−H)⁻.

¹H-NMR (200 MHz, DMSO-d₆): δ 1.43-1.57 (2H, m), 1.77-1.92 (2H, m), 3.00-3.09 (2H, m) 3.54 (2H, s), 4.33 (2H, d, J=5.5 Hz), 4.59-4.70 (1H, m), 4.99 (2H, s), 7.12-7.32 (10H, m), 7.70-7.93 (2H, m), 8.11 (1H, d, J=7.5 Hz), 8.1 9 (2H, d, J=8.5 Hz), 8.60 (1H, d, J=8.5 Hz), 8.72-8.8 6 (2H, m), 12.3 (1H, br).

### EXAMPLE 124-1

### Ethyl{2-[({(2S)-5-{[(benzyloxy)carbonyl]amino}-2-[(tert-butoxycarbonyl)amino]pentanoyl}amino)-methyl]phenyl}acetate

The target compound was obtained in a similar manner to that of Example 42-1.

MS ((+)ESI) m/z: 564 (M+Na)⁺.

### EXAMPLE124-2

### Ethyl(2-{[((2S)-2-amino-5-{(benzyloxy)carbonyl]-amino}pentanoyl)amino]methyl}phenyl)acetate hydrochloride

The target compound was obtained in a similar manner to that of Example 82-1.

MS ((+)ESI) m/z: 442 (M+H)⁺.

### EXAMPLE 124-3

### Ethyl(2-{[((2S)-5-{[(benzyloxy)carbonyl]amino}-2-{[(1-methyl-1H-indol-2-yl)carbonyl]amino)-pentanoyl)amino]methyl}phenyl)acetate

The target compound was obtained in a similar manner to that of Example 27-3.

MS ((+)ESI) m/z: 621 (M+Na)⁺.

### EXAMPLE 125

### (2-{[((2S)-5-{[(Benzyloxy)carbonyl]amino}-2-{[(1-methyl-1H-indol-2-yl)carbonyl]amino}pentanoyl)-amino]methyl}phenyl)acetic acid

The target compound was obtained in a similar manner to that of Example 28.

MS ((−)ESI) m/z: 569 (M−H)⁻.

¹H-NMR (200 MHz, DMSO-d₆): δ 1.43-1.61 (2H, m), 1.71-1.85 (2H, m), 3.00-3.09 (2H, m), 3.66 (2H, s), 3.97 (3H, s), 4.31 (2H, d, J=5.5 Hz), 4.39-4.49 (1H, m), 5.00 (2H, s), 7.07-7.33 (13H, m), 7.53 (1H, d, J=8.5 Hz), 7.64 (1H, d, J=8.0 Hz), 8.40 (1H, t, J=6.0 Hz), 8.50 (1H, d, J=8.0 Hz), 12.4 (1H, br).

### EXAMPLE 126

### Ethyl{2-[({(2S)-5-{(benzyloxy)carbonyl]amino}-2-[(2-quinolinylcarbonyl)amino]pentanoyl}amino)-methyl]phenyl}acetate

The target compound was obtained in a similar manner to that of Example 27-3.

MS ((+)ESI) m/z: 619 (M+Na)⁺.

### EXAMPLE 127

### {2-[({(2S)-5-{[(Benzyloxy)carbonyl]amino}-2-[(2-quinolinylcarbonyl)amino]pentanoyl}amino)methyl]-phenyl}acetic acid

The target compound was obtained in a similar manner to that of Example 28.

MS ((−)ESI) m/z: 567 (M−H)⁻.

¹H-NMR (200 MHz, DMSO-d₆): δ 1.41-1.56 (2H, m), 1.76-1.91 (2H, m), 2.98-3.08 (2H, m), 3.67 (2H, s), 4.34 (2H, d, J=60 Hz), 4.58-4.69 (1H, m), 4.98 (2H, s), 7.20-7.32 (10H, m), 7.70-7.93 (2H, m), 8.10 (1H, d, J=7.5 Hz), 8.18 (2H, d, J=8.5 Hz), 8.58-8.68 (2H, m), 8.83 (1H, d, J=8.5 Hz), 12.4 (1H, br).

### EXAMPLE 128

### (5-Methyl-2-oxc-1,3-dioxol-4-yl)methyl 6-[((2S)-2-(1-benzofuran-2-ylcarbonyl)amino]-5-{(benzyl-oxy)carbonyl]amino}pentanoyl)amino]hexanoate

To a solution of sodium 6-[[(2S)-2-[(1-benzofuran-2-ylcarbonyl) amino]-5-[(benzyloxy)-carbonyl}amino]pentanoyl]amino]hexanoate (150 mg) in N,N-dimethylacetamide (1.5 mL), was added 4-(bromomethyl)-5-methyl-1,3-dioxol-2-one (37.5 μL) The mixture was stirred at room temperature for 20 hours The mixture was diluted with water (10 mL) and extracted with ethyl acetate (10 mL). The organic layer was washed with water (10 mL×2) and brine (10 mL), and dried over magnesium sulfate. Filtration followed by evaporation gave the target compound (93 mg) as a white solid.

MS ((+)ESI) m/z: 658 (M+Na)⁺.

¹H-NMR (200 MHz, DMSO-d₆): δ 1.17-1.82 (10H, m), 2.14 (3H, s), 2.33 (2H, t, J=7.0 Hz), 2.97-3.10 (4H, m), 4.35-4.46 (1H, m), 4.93 (2H, s), 5.00 (2H, s), 7.22-7.52 (8H, m), 7.63 (1H, s), 7.68 (1H, d, J=8.5 Hz), 7.78 (1H, d, J=7.0 Hz), 8.03 (1H, t, J=5.5 Hz), 8.50 (1H, d, J=8.0 Hz).

### EXAMPLE 129

### [(2,2-Dimethylpropanoyl)oxy]methyl 6-[((2S)-2-[(1-benzofuran-2-ylcarbonyl)amino]-5-{[(benzyloxy)-carbonyl]amino}pentanoyl)amino]hexanoate

The target compound was obtained in a similar manner to that of Example 128.

MS ((+) ESI) m/z: 660 (M+Na)⁺.

¹H-NMR (200 MHz, DMSO-d₆): δ 1.13 (9H, s), 1.20-1.80 (10H, m), 2.34 (2H, t, J=7.0 Hz), 2.96-3.10 (4H, m), 4.35-4.46 (1H, m), 5.00 (2H, s), 5.68 (2H, s), 7.24-7.52 (8H, m), 7.62 (1H, s), 7.69 (1H, d, J=8.5 Hz), 7.78 (1H, d, J=7.0 Hz), 8.03 (1H, t, J=5.5 Hz), 8.50 (1H, d, J=8.0 Hz).

### EXAMPLE 130

### 1-{[(Cyclohexyloxy)carbonyl]oxy}ethyl 6-[((2S)-2-[(1-benzofuran-2-ylcarbonyl)amino]-5-{[(benzyloxy)carbonyl]amino}pentanoyl)amino]hexanoate

The target compound was obtained in a similar manner to that of Example 128.

MS ((+)ESI) m/z: 716 (M+Na)⁺.

¹H-NMR (200 MHz, DMSO-d₆): δ 1.16-1.87 (23H, m), 2.31 (2H, t, J=7.0 Hz), 2.96-3.09 (4H, m), 4.33-4.63 (2H, m), 5.00 (2H, s), 6.62 (1H, q, J=5.0 Hz), 7.24-7.52 (8H, m), 7.62 (1H, s), 7.69 (1H, d, J=8.5 Hz), 7.78 (1H, d, J=7.5 Hz), 8.03 (1H, t, J=5.5 Hz), 8.50 (1H, d, J=8.0 Hz).

### EXAMPLE 131

### Methyl 3-{2-[((2S)-5-{[(benzyloxy)carbonyl]amino}-2-{[(1-methyl-1H-indol-3-yl)carbonyl]amino}-pentanoyl)amino]phenyl}propanate

To a solution of methyl 3-[2-[[(2S)-2-amino-5-[[(benzyloxy)carbonyl]amino]pentanoyl]-amino]phenyl]propanoate hydrochloride (208 mg) and 1-hydroxybenzotriazole (160 mg) in N,N-dimethylformamide (5.0 mL), was added 1-(3-dimethylaminopropyl)-3-ethylcarbodiimide (184 mg) at 5° C. under nitrogen. The mixture was stirred at room temperature for 12 hours.

The resulting mixture was poured into water and the aqueous layer was extracted with ethyl acetate. The organic layer was washed successively with saturated aqueous sodium bicarbonate three times and brine, dried over anhydrous magnesium sulfate, and evaporated under reduced pressure. The residue was purified by column chromatography on silica gel (hexane/ethyl acetate=1:1 to 1:2) to give the target compound (357 mg).

MS ((+)ESI) m/z: 607 (M+Na)⁺.

### EXAMPLE 132

### 3-{2-[((2S)-5-{[(Benzyloxy)carbonyl]amino}-2-{[(1-methyl-1H-indol-3-yl)carbonyl]amino}pentanoyl)-amino]phenyl}propanoic acid

To a solution of methyl 3-[2-[[(2S)-5-[[(benzyloxy)carbonyl]amino]-2-[(1-methyl-1H-indol-3-yl) carbonyl]amino]pentanoyl]-amino]phenyl]propanoic acid (355 mg) obtained in Example 131 in 1,4-dioxane (10 mL), was added 1N sodium hydroxide (1.82 mL) at room temperature. The mixture was stirred at 45° C. for 2.5 hours. The resulting mixture was poured into 1N hydrochloric acid and the aqueous layer was extracted with a mixture of chloroform and methanol (5:1). The organic layer was dried over anhydrous magnesium sulfate, evaporated, and dried in vacuo to give the target compound (373 mg).

MS ((−)ESI) m/z: 569 (M−H)⁻.

¹H-NMR (DMSO-d₆): δ 1.5-1.95 (4H, m), 2.4-2.5 (2H, m), 2.75-2.9 (2H, m), 3.0-3.15 (2H, m), 3.84 (3H, s), 4.6-4.8 (1H, m), 5.00 (2H, s), 7.05-7.55 (11H, m), 7.95-8.05 (1H, m), 8.1-8.1 (2H, m).

### EXAMPLE 133

### Methyl 3-{2-[((2S)-5-{[(benzyloxy)carbonyl]amino}-2-{[(1-methyl-1H-indazol-3-yl)carbonyl]amino}-pentanoyl)amino]phenyl}propanoate

The target compound was obtained in a similar manner to that of Example 131.

MS ((+) ESI) m/z: 608 (M+Na)⁺.

### EXAMPLE 134

### 3-{2-[((2S)-5-{[(Benzyloxy)carbonyl]amino}-2-{[(1-methyl-1H-indazol-3-yl)carbonyl]amino}pentanoyl)-amino]phenyl}propanoic acid

The target compound was obtained in a similar manner to that of Example 132.

MS ((−)ESI) m/z: 570 (M−H)⁻.

¹H-NMR (DMSO-d₆): δ 1.45-1.65 (2H, m), 1.85-2.0 (2H, m), 2.4-2.5 (2H, m), 2.75-2.9 (2H, m), 3.0-3.15 (2H, m), 4.15 (3H, s), 4.7-4.95 (1H, m), 4.99 (2H, s), 7.1-7.55 (10H, m), 7.7-7.8 (1H, m), 8.1-8.5 (2H, m).

### EXAMPLE 135

### Methyl 3-{2-[((2S)-5-{[(benzyloxy)carbonyl]amino}-2-{[(8-methylimidazo[1,2-a]pyridin-2-yl)carbonyl]-amino}pentanoyl)amino]phenyl}propanoate

The target compound was obtained in a similar manner to that of Example 131.

MS ((+)ESI) m/z: 608 (M+Na)⁺.

### EXAMPLE 136

### 3-{2-[((2S)-5-{[(Benzyloxy)carbonyl]amino}-2-{[(8-methylimidazo[1,2-a]pyridin-2-yl)carbonyl]amino}-pentanoyl)amino]phenyl}propanoic acid

The target compound was obtained in a similar manner to that of Example 132.

MS ((−)ESI) m/z: 570 (M−H)⁻.

¹H-NMR (DMSO-d₆): δ 1.5-2.0 (4H, m), 2.4-2.5 (2H, m), 2.58 (3H, s), 2.75-2.9 (2H, m), 3.0-3.2 (2H, m), 4.75-4.9 (1H, m), 5.00 (2H, s) 7.1-7.55 (10H, m), 8.6-8.9 (3H, m).

### EXAMPLE 137

### Methyl 3-(2-{[(2S)-5-{[(benzyloxy)carbonyl]amino}-2-(2-naphthoylamino)pentanoyl]amino}phenyl)-propanoate

The target compound was obtained in a similar manner to that of Example 131.

MS ((+)ESI) m/z: 604 (M+Na)⁺.

### EXAMPLE 138

### 3-(2-{[(2S)-5-{[(Benzyloxy)carbonyl]amino}-2-(2-naphthoylamino)pentanoyl]amino}phenyl)propanoic acid

The target compound was obtained in a similar manner to that of Example 132.

MS ((−)ESI) m/z: 566 (M−H)⁻.

¹H-NMR (DMSO-d₆): δ 1.5-1.75 (2H, m), 1.8-2.0 (2H, m), 2.45-2.6 (2H, m), 2.75-2.9 (2H, m), 3.05-3.2 (2H, m), 4.6-4.8 (1H, m), 5.01 (2H, s), 7.1-7.45 (9H, m), 7.55-7.7 (2H, m), 7.9-8.1 (4H, m), 8.56 (1H, s).

### EXAMPLE 139

### Methyl 3-[2-({(2S)-5-{(benzyloxy)carbonyl]amino}-2-[(3-quinolinylcarbonyl) amino]pentanoyl}amino)-phenyl]propanoate

The target compound was obtained in a similar manner to that of Example 131.

MS ((+)ESI) m/z: 605 (M+Na)⁺.

### EXAMPLE 140

### 3-[2-({(2S)-5-{[(Benzyloxy)carbonyl]amino}-2-[(3-quinolinylcarbonyl)amino]pentanoyl}amino)phenyl]-propanoic acid

The target compound was obtained in a similar manner to that of Example 132.

MS ((−)ESI) m/z: 567 (M−H)⁻.

¹H-NMR (DMSO-d₆): δ 1.5-2.1 (4H, m), 2.35-2.6 (2H, m), 2.7-2.9 (2H, m), 2.95-3.2 (2H, m), 4.6-4.8 (1H, m), 5.01 (2H, s), 7.05-7.5 (9H, m), 7.65-7.75 (1H, m), 7.8-7.95 (1H, m), 8.05-8.2 (2H, m), 9.04 (1H, S), 9.35 (1H, m).

### EXAMPLE 141

### Methyl 3-[2-({(2S)-5-{[(benzyloxy)carbonyl]amino}-2-[(3-isoquinolinylcarbonyl)amino]pentanoyl}-amino)phenyl]propanoate

The target compound was obtained in a similar manner to that of Example 131.

MS ((+)ESI) m/z: 605 (M+Na)⁺.

### EXAMPLE 142

### 3-[2-({(2S)-5-{[(Benzyloxy)carbonyl]amino}-2-[(3-isoquinolinylcarbonyl)amino]pentanoyl}amino)-phenyl]propanoic acid

The target compound was obtained in a similar manner to that of Example 132.

MS ((−)ESI) m/z: 567 (M−H)⁻.

¹H-NMR (DMSO-d₆): δ 1.5-1.7 (2H, m), 1.8-2.05 (2H, m), 2.4-2.55 (2H, m), 2.75-2.9 (2H, m), 3.0-3.2 (2H, m), 4.8-4.95 (1H, m), 4.98 (2H, s), 7.1-7.45 (9H, m), 7.75-7.95 (2H, m), 8.15-8.35 (2H, m), 8.61 (1H, s), 9.79 (1H, s).

### EXAMPLE 143

### Methyl 3-[2-({(2S)-5-{[(benzyloxy)carbonyl]amino}-2-[(2-quinoxalinylcarbonyl)amino]pentanoyl}amino)-phenyl]propanoate

The target compound was obtained in a similar manner to that of Example 131.

MS ((+)ESI) m/z: 606 (M+Na)⁺.

### EXAMPLE 144

### 3-2-({(2S)-5-{(Benzyloxy)carbonyl]amino}-2-(2-quinoxalinylcarbonyl)amino]pentanoyl}amino)-phenyl]propanoic acid

The target compound was obtained in a similar manner to that of Example 132.

MS ((−)ESI) m/z: 568 (M−H)⁻.

¹H-NMR (DMSO-d6): δ 1.5-1.7 (2H, m), 1.85-2.1 (2H, m), 2.4-2.5 (2H, m), 2.75-2.9 (2H, m), 3.05-3.2 (2H, m), 4.75-4.9 (1H, m), 4.99 (2H, s), 7.1-7.45 (9H, m), 7.95-8.05 (2H, m), 8.2-8.35 (2H, m), 9.51 (1H, s).

### EXAMPLE 145

### Methyl 3-[2-({(2S)-5-{[(benzyloxy)carbonyl]amino}-2-[(4-quinolinylcarbonyl)amino]pentanoyl}amino)-phenyl]propanoate

The target compound was obtained in a similar manner to that of Example 131.

MS ((+)ESI) m/z: 605 (M+Na)⁺.

### EXAMPLE 146

### 3-[2-({(2S)-5-{[(Benzyloxy)carbonyl]amino}-2-[(4-quinolinylcarbonyl)amino]pentanoyl}amino)phenyl]-propanoic acid

The target compound was obtained in a similar manner to that of Example 132.

MS ((−)ESI) m/z: 567 (M−H)⁻.

¹H-NMR (DMSO-d₆): δ 1.55-2.0 (4H, m), 2.45-2.6 (2H, m), 2.8-2.95 (2H, m), 3.05-3.2 (2H, m), 4.65-4.8 (1H, m), 5.01 (2H, s), 7.1-7.4 (9H, m), 7.55-7.7 (2H, m), 7.75-7.9 (1H, m), 8.05-8.1 (1H, m), 8.2-8.25 (1H, m), 8.95-9.0 (1H, m).

### EXAMPLE 147

### Methyl 3-[2-({(2S)-5-{[(benzyloxy)carbonyl]amino}-2-[(1-isoquinolinylcarbonyl) amino]pentanoyl}-amino)phenyl]propanoate

The target compound was obtained in a similar manner to that of Example 131.

MS ((+)ESI) m/z: 605 (M+Na)⁺.

### EXAMPLE 148

### 3-[2-({(2S)-5-{(Benzyloxy)carbonyl]amino}-2-(1-isoquinolinylcarbonyl)amino]pentanoyl}amino)-phenyl]propanoic acid

The target compound was obtained in a similar manner to that of Example 132.

MS ((−)ESI) m/z: 567 (M−H)⁻.

¹H-NMR (DMSO-d₆): δ 1.5-2.05 (4H, m), 2.4-2.55 (2H, m), 2.75-2.9 (2H, m), 3.05-3.2 (2H, m), 4.7-4.9 (1H, m), 4.99 (2H, s), 7.1-7.45 (9H, m), 7.7-7.9 (2H, m), 8.0-8.1 (2H, m), 8.55-8.6 (1H, m), 8.95-9.05 (1H, m).

### EXAMPLE 149

### Methyl 3-{2-[((2S)-5-{[(benzyloxy)carbonyl]amino}-2-{[5-(4-chlorophenyl)-2-furoyl]amino}pentanoyl)-amino]phenyl}propanoate

The target compound was obtained in a similar manner to that of Example 131.

MS ((+)ESI) m/z: 654, 656 (M+Na)⁺.

### EXAMPLE 150

### 3-{2-[((2S)-5-{[(Benzyloxy)carbonyl]amino}-2-{[5-(4-chlorophenyl)-2-furoyl]amino}pentanoyl) amino]-phenyl}propanoic acid

The target compound was obtained in a similar manner to that of Example 132.

MS ((−)ESI) m/z: 616, 618 (M−H)⁻.

¹H-NMR (DMSO-d₆): δ 1.5-2.0 (4H, m), 2.45-2.55 (2H, m), 2.75-2.9 (2H, m), 3.0-3.2 (2H, m), 4.6-4.75 (1H, m), 7.1-7.4 (11H, m), 7.5-7.6 (2H, m), 7.9-8.0 (2H, m).

### EXAMPLE 151

### Methyl 3-[2-({(2S)-5-{[(benzyloxy)carbonyl]amino}-2-[(2-biphenylylcarbonyl)amino]pentanoyl}amino)-phenyl]propanoate

The target compound was obtained in a similar manner to that of Example 131.

MS ((+)ESI) m/z: 630 (M+Na)⁺.

### EXAMPLE 152

### 3-[2-({(2S)-5-{[(Benzyloxy)carbonyl]amino}-2-[(2-biphenylylcarbonyl)amino]pentanoyl}amino)phenyl]-propanoic acid

The target compound was obtained in a similar manner to that of Example 132.

MS ((−)ESI) m/z: 592 (M−H)⁻.

¹H-NMR (DMSO-d₆): δ 1.2-1.8 (4H, m), 2.4-2.55 (2H, m), 2.7-2.85 (2H, m), 2.9-3.05 (2H, m), 4.35-4.5 (1H, m), 5.02 (2H, s), 7.1-7.6 (18H, m).

### EXAMPLE 153

### Methyl 3-[2-({(2S)-5-{(benzyloxy)carbonyl]amino}-2-[(4-phenoxybenzoyl)amino]pentanoyl}amino)-phenyl]propanoate

The target compound was obtained in a similar manner to that of Example 131.

MS ((+)ESI) m/z: 646 (M+Na)⁺.

### EXAMPLE 154

### 3-[2-({(2S)-5-{[(Benzyloxy)carbonyl]amino}-2-[(4-phenoxybenzoyl)amino]pentanoyl}amino)phenyl]-propanoic acid

The target compound was obtained in a similar manner to that of Example 132.

MS ((−)ESI) m/z: 608 (M−H)⁻.

¹H-NMR (DMSO-d₆): δ 1.45-1.7 (2H, m), 1.75-1.95 (2H, m), 2.4-2.6 (2H, m), 2.75-2.9 (2H, m), 3.0-3.2 (2H, m), 4.5-4.7 (1H, m), 5.00 (2H, s), 7.0-7.5 (16H, m), 7.9-8.0 (2H, m).

### EXAMPLE 155

### Methyl 3-[2-({(2S)-5-{(benzyloxy)carbonyl]amino}-2-[(3,4-dimethoxybenzoyl)amino]pentanoyl}amino)-phenyl]propanoate

The target compound was obtained in a similar manner to that of Example 131.

MS ((+)ESI) m/z: 614 (M+Na)⁺.

### EXAMPLE 156

### 3-[2-({(2S)-5-{[(Benzyloxy)carbonyl]amino}-2-[(3,4-dimethoxybenzoyl)amino]pentanoyl}amino)-phenyl]propanoic acid

The target compound was obtained in a similar manner to that of Example 132.

MS ((−)ESI) m/z: 576 (M−H)⁻.

¹H-NMR (DMSO-d₆): δ 1.45-1.7 (2H, m), 1.75-2.0 (2H, m), 2.4-2.55 (2H, m), 2.75-2.9 (2H, m), 3.0-3.2 (2H, m), 3.81 (6H, s), 4.55-4.75 (1H, m), 5.00 (2H, s), 7.0-7.45 (10H, m), 7.5-7.65 (2H, m).

### EXAMPLE 157

### Methyl 3-{2-[((2S)-5-{[(benzyloxy)carbonyl]-amino}-2-{(6-methyl-2-pyridinyl) carbonyl]amino}-pentanoyl)amino]phenyl}propanoate

The target compound was obtained in a similar manner to that of Example 131.

MS ((+)ESI) m/z: 569 (M+Na)⁺.

### EXAMPLE 158

### 3-{2-[((2S)-5-{[(Benzyloxy)carbonyl]amino}-2-{[(6-methyl-2-pyridinyl)carbonyl]amino}pentanoyl)-amino]phenyl}propanoic acid

The target compound was obtained in a similar manner to that of Example 132.

MS ((−)ESI) m/z: 531 (M−H)⁻.

¹H-NMR (DMSO-d₆): δ 1.4-1.65 (2H, m); 1.7-2.0 (2H, m), 2.4-2.55 (2H, m), 2.57 (3H, s), 2.75-2.9 (2H, m), 3.0-3.15 (2H, m) 4.7-4.9 (1H, m), 4.99 (2H, m), 7.1-7.55 (10H, m), 7.85-7.95 (2H, m).

### EXAMPLE 159

### Methyl 3-[2-({(2S)-5-{[(benzyloxy)carbonyl]amino}-2-(3,4-dimethylbenzoyl)amino]pentanoyl}amino)-phenyl]propanoate

The target compound was obtained in a similar manner to that of Example 131.

MS ((+)ESI) m/z: 582 (M+Na)⁺.

### EXAMPLE 160

### 3-[2-({(2S)-5-{(Benzyloxy)carbonyl]amino}-2-[(3,4-dimethylbenzoyl)amino]pentanoyl}amino)-phenyl]propanoic acid

The target compound was obtained in a similar manner to that of Example 132.

MS ((−)ESI) m/z: 544 (M−H)⁻.

¹H-NMR (DMSO-d₆): δ 1.45-1.7 (2H, m), 1.75-1.95 (2H, m), 2.27 (6H, s), 2.4-2.5 (2H, m), 2.75-2.9 (2H, m), 2.95-3.15 (2H, m), 4.5-4.7 (1H, m) r 5.00 (2H, s), 7.05-7.45 (10H, m), 7.6-7.8 (2H, m).

### EXAMPLE 161

### Methyl 3-[2-({(2S)-5-{[(benzyloxy)carbonyl]amino}-2-[(3,4-dichlorobenzoyl)amino]pentanoyl}amino)-phenyl]propanoate

The target compound was obtained in a similar manner to that of Example 131.

MS ((+)ESI) m/z: 622, 624 (M+Na)⁺.

### EXAMPLE 162

### 3-[2-({(2S)-5-{[(Benzyloxy)carbonyl]amino}-2-[(3,4-dichlorobenzoyl)amino]pentanoyl}amino)-phenyl]propanoic acid

The target compound was obtained in a similar manner to that of Example 132

MS ((−)ESI) m/z: 584, 586 (M−H)⁻.

¹H-NMR (DMSO-d₆): δ 1.45-1.7 (2H, m), 1.75-1.95 (2H, m), 1.4-1.55 (2H, m), 2.75-2.9 (2H, m), 3.0-3.15 (2H, m), 4.5-4.7 (1H, m), 5.01 (2H, s), 7.1-7.4 (9H, m), 7.76 (1H, d, J=8.3 Hz), 7.91 (1H, dd, J=1.9, 8.4 Hz), 8.20 (1H, d, J=1.9 Hz).

### EXAMPLE 163

### Methyl 3-[2-({(2S)-5-({(2-chlorobenzyl)oxy]-carbonyl}amino)-2-[(1H-indol-2-ylcarbonyl)amino]-pentanoyl}amino)phenyl]propanoate

The target compound was obtained in a similar manner to that of Example 131.

MS ((−)ESI) m/z: 603, 605 (M−H)⁻.

### EXAMPLE 164

### 3-[2-({(2S)-5-({[(2-Chloroberizyl)oxy]carbonyl}-amino)-2-[(1H-indol-2-ylcarbonyl)amino]pentanoyl}-amino)phenyl]propanoic acid

The target compound was obtained in a similar manner to that of Example 132.

MS ((−)ESI) m/z: 589, 591 (M−H)⁻.

¹H-NMR (DMSO-d₆): δ 1.5-2.0 (4H, m), 2.4-2.6 (2H, m), 2.75-2.9 (2H, m), 3.0-3.2 (2H, m), 4.6-4.8 (1H, m), 5.09 (2H, s), 7.0-7.55 (12H, m), 7.62 (1H, d, J=7.8 Hz).

### EXAMPLE 165

### Methyl 3-{2-((2S)-5-({(2-chlorobenzyl)oxy]-carbonyl}amino)-2-{[(1-methyl-1H-indol-2-yl)-carbonyl]amino}pentanoyl) amino]phenyl}propanoate

The target compound was obtained in a similar manner to that of Example 131.

MS ((+)ESI) m/z: 641, 643 (M+Na)⁺.

### EXAMPLE 166

### 3-{2-[((2S)-5-({[(2-Chlorobenzyl)oxy]carbonyl}-amino)-2-{[(1-methyl-1H-indol-2-yl)carbonyl]-amino}pentanoyl)amino]phenyl }propanoic acid

The target compound was obtained in a similar manner to that of Example 132.

MS ((−) ESI) m/z: 603,605 (M−H)⁻.

¹H-NMR (DMSO-d6): δ 1.45-2.05 (4H, m), 2.3-2.6 (2H, m), 2.75-2.9 (2H, m), 3.0-3.2 (2H, m), 3.98 (3H, s), 4.5-4.7 (1H, m), 5.09 (2H, s), 7.05-7.7 (13H, m).

### EXAMPLE 167

### Methyl 3-(2-{[(2S)-2-[(4-biphenylylcarbonyl)-amino]-5-({(2-chlorobenzyl)oxy]carbonyl}amino)-pentanoyl]amino}phenyl)propanoate

The target compound was obtained in a similar manner to that of Example 131.

MS ((+)ESI) m/z: 664, 666 (M+Na)⁺.

### EXAMPLE 168

### 3-(2-{[(2S)-2-[(4-Biphenylylcarbonyl)amino]-5-({[(2-chlorobenzyl)oxy]carbonyl}amino)pentanoyl]-amino}phenyl)propanoic acid

The target compound was obtained in a similar manner to that of Example 132.

MS ((−)ESI) m/z: 626, 628 (M−H)⁻.

¹H-NMR (DMSO-d₆): δ 1.5-2.0 (4H, m), 2.4-2.6 (2H, m), 2.75-2.9 (2H, m), 3.05-3.2 (2H, m), 4.55-4.75 (1H, m), 5.09 (2H, s), 7.1-7.6 (11H, m), 7.7-7.85 (4H, m), 8.03 (2H, d, J=8.3 Hz).

### EXAMPLE 169-1

### Ethyl 2′-nitro-3-biphenylylcarboxylate

To a solution of 1-iodo-2-nitrobenzene (2.0 g) and [3-(ethoxycarbonyl)phenyl]boronic acid (2.0 g) in 1,2-dimethoxyethane (20 mL), were added tetrakis(triphenyl)palladium(0) (0.93 g) and 2M sodium carbonate (8.4 mL) at room temperature. The mixture was stirred at 80° C. for 18 hours.

The resulting mixture was poured into water and the aqueous layer was extracted with ethyl acetate. The organic layer was washed successively with saturated aqueous sodium bicarbonate and brine, dried over anhydrous magnesium sulfate, and evaporated under reduced pressure. The residue was purified by column chromatography on silica gel (hexane/ethyl acetate=10:1 to 5:1) to give the target compound (1.2 g)

MS ((+)ESI) m/z: 294 (M+Na)⁺.

### EXAMPLE 169-2

### Ethyl 2′-amino-3-biphenylylcarboxylate

To a solution of ethyl 2′-nitro-3-biphenylylcarboxylate (1.1 g) obtained in Example 169-1 in a mixture of ethanol (15 mL) and water (5 mL), were added iron (679 mg) and ammonium chloride (108 mg) at room temperature under nitrogen. The mixture was refluxed for 1 hour.

The resulting mixture was filtered through celite, and the filtrate was evaporated under reduced pressure. The residue was dissolved into a mixture of saturated aqueous sodium bicarbonate and ethyl acetate. After separation, the organic layer was washed with brine, dried over anhydrous magnesium sulfate, evaporated, and dried in vacuo to give the target compound (1.0 g).

MS ((+)ESI) m/z: 242 (M+H)⁺.

### EXAMPLE 169-3

### Ethyl 2-({(2S)-5-{[(benzyloxy) carbonyl]amino}-2-[(tert-butoxycarbonyl)amino}pentanoyl}amino)-3-biphenylylcarboxylate

To a solution of (2S)-5-[[(benzyloxy)-carbonyl]amino]-2-[(tert-butoxycarbonyl) amino]-pentanoic acid (305 mg) and ethyl 2′-amino-3-biphenylylcarboxylate (254 mg) obtained in Example 169-2 in dichloromethane (7 mL), were added bromotripyrrolidinophosphonium hexafluorophosphate (490 mg) and N,N-diisopropylethylamine (370 mg) at 5° C. under nitrogen. The mixture was stirred at room temperature for 12 hours.

The resulting mixture was poured into 1N hydrochloric acid and the aqueous layer was extracted with ethyl acetate. The organic layer was washed successively with saturated aqueous sodium bicarbonate and brine, dried over anhydrous magnesium sulfate, and evaporated under reduced pressure. The residue was purified by column chromatography on silica gel (hexane/ethyl acetate=2:1 to 4:3) to give the target compound (357 mg).

MS ((+)ESI) m/z: 612 (M+Na)⁺.

### EXAMPLE 169-4

### Ethyl 2′-[((2S)-2-amino-5-{[(benzyloxy)carbonyl]-amino}pentanoyl)amino]-3-biphenylylcarboxylate hydrochloride

To a solution of ethyl 2-(2S)-5-(benzyloxy)-carbonyl]amino]-2-[(tert-butoxycarbonyl)amino]-pentanoyl]amino]-3-biphenylylcarboxylate (292 mg) obtained in Example 169-3 in ethyl acetate (2 mL), was added hydrogen chloride (4N in ethyl acetate, 5 mL) at room temperature under nitrogen. The mixture was stirred at the same temperature for 2 hours. The resulting mixture was evaporated dried invacuo to give the target compound (281 mg).

MS ((+)ESI) m/z: 490 (M−HCl+H)⁺.

### EXAMPLE 169-5

### Ethyl 2′-((2S)-2-[(1-benzofuran-2-ylcarbonyl)-amino]-5-{[(benzyloxy)carbonyl]amino}pentanoyl)-amino]-3-biphenylylcarboxylate

The target compound was obtained in a similar manner to that of Example 131.

MS ((+)ESI) m/z: 656 (M+Na)⁺.

### EXAMPLE 170

### 2′-[((2S)-2-[(1-Benzofuran-2-ylcarbonyl)amino]-5-{[(benzyloxy)carbonyl}amino}pentanoyl)amino]-3-biphenylylcarboxylic acid

The target compound was obtained in a similar manner to that of Example 132.

MS ((−)ESI) m/z: 604 (M−H)⁻.

¹H-NMR (DMSO-d₆): δ 1.3-1.8 (4H, m), 2.9-3.1 (2H, m), 4.4-4.6 (1H, m), 4.9 9 (2H, s), 7.15-7.9 (18H, m).

### EXAMPLE 171-1

### Methyl 2′-nitro-4-biphenylylcarboxylate

The target compound was obtained in a similar manner to that of Example 169-1.

MS ((+)ESI) m/z: 280 (M+Na)⁺.

### EXAMPLE 171-2

### Methyl 2′-amino-4-biphenylylcarboxylate

The target compound was obtained in a similar manner to that of Example 169-2.

MS ((+)ESI) m/z: 228 (M+H)⁺.

### EXAMPLE 171-3

### Methyl 2′-({(2S)-5-{[(benzyloxy)carbonyl]amino}-2-[(tert-butoxycarbonyl)amino]pentanoyl}amino)-4-biphenylylcarboxylate

To a solution of (2S)-5-[[(benzyloxy)-carbonyl]amino]-2-[(tert-butoxycarbonyl)amino]-pentanoic acid (300 mg) and ethyl 2′-amino-4-biphenylylcarboxylate (232 mg) in dichloromethane (10 mL), were added O-(7-azabenzotriazol-1-yl)-N,N,N′,N′-tetramethyluronium hexafluorophosphate (342 mg) and N,N-diisopropylethylamine (317 mg) at 5° C. under nitrogen. The mixture was stirred at room temperature for 12 hours.

The resulting mixture was poured into 1N hydrochloric acid and the aqueous layer was extracted with ethyl acetate. The organic layer was washed successively with water, saturated aqueous sodium bicarbonate and brine, dried over anhydrous magnesium sulfate, and evaporated under reduced pressure. The residue was purified by column chromatography on silica gel (hexane/ethyl acetate=2:1 to 1:1) to give the target compound (433 mg).

MS ((+)ESI) m/z: 598 (M+Na)⁺.

### EXAMPLE 171-4

### Methyl 2′-[((2S)-2-amino-5-{[(benzyloxy)carbonyl]-amino}pentanoyl)amino]-4-biphenylylcarboxylate hydrochloride

The target compound was obtained in a similar manner to that of Example 169-4.

MS ((+)ESI) m/z: 484 (M−HCl+Na)⁺.

### EXAMPLE 171-5

### Methyl 2′-[((2S)-2-[(1-benzofuran-2-ylcarbonyl)-amino]-5-{[(benzyloxy)carbonyl]amino}pentanoyl)-amino]-4-biphenylylcarboxylate

The target compound was obtained in a similar manner to that of Example 131.

MS ((+)ESI) m/z: 642 (M+Na)⁺.

### EXAMPLE 172

### 2′-[((2S)-2-[(1-Benzofuran-2-ylcarbonyl)amino]-5-{[(benzyloxy)carbonyl]amino}pentanoyl)amino]-4-biphenylylcarboxylic acid

The target compound was obtained in a similar manner to that of Example 132.

MS ((−)ESI) m/z: 604 (M−H)⁻.

¹H-NMR (DMSO-d₆): δ 1.3-1.85 (4H, m), 2.9-3.1 (2H, m), 4.4-4.6 (1H, m), 4.99 (2H, s), 7.2-7.75 (15H, m), 7.78 (1H, d, J=7.6 Hz), 7.94 (1H, d, J=8.1 Hz).

### EXAMPLE 173-1

### tert-Butyl[(2-aminophenyl)thio]acetate

To a suspension of sodium hydride (60% in oil, 703 mg) in N,N-dimethylformamide (40 mL), was added 2-aminobenzenethiol (2.0 g) dropwise at 5° C. under nitrogen. The mixture was stirred at the same temperature for 40 minutes. To this one was added tert-butyl bromoacetate (3.4 g), and the mixture was stirred at 5° C. for 30 minutes.

The resulting mixture was poured into water and the aqueous was extracted with ethyl acetate. The organic layer was washed successively with water two times, saturated aqueous sodium bicarbonate and brine, dried over an hydrous magnesium sulfate, and evaporated under reduced pressure. The residue was purified by column chromatography on silica gel (hexane/ethyl acetate=10:1 to 5:1) to give the target compound (3.5 g)

MS ((+)ESI) m/z: 262 (M+Na)⁺.

### EXAMPLE 173-2

### tert-Butyl{[2-({(2S)-5-{[(benzyloxy)carbonyl]-amino}-2-[(tert-butoxycarbonyl)amino]pentanoyl}-amino)phenyl]thio}acetate

The target compound was obtained in a similar manner to that of Example 171-3.

MS ((+)ESI) m/z: 610 (M+Na)⁺.

### EXAMPLE 173-3

### Methyl({2-[((2S)-2-amino-5-{[(benzyloxy)-carbonyl]amino}pentanoyl)amino]phenyl}thio)acetate hydrochloride

To a solution of tert-butyl [[2-[[(2S)-5-[[(benzyloxy)carbonyl]amino]-2-[(tert-butoxy-carbonyl)amino]pentanoyl]amino]phenyl]thio}acetate (1.25 g) obtained in Example 172-2 in dichloromethane (12.5 mL), was added trifluoroacetic acid (2.5 mL) at room temperature under nitrogen. The mixture was stirred at the same temperature for 24 hours.

The resulting mixture was evaporated and dried in vacuo. Thionyl chloride (380 mg) was added to methanol (6.3 mL) dropwise at 5° C. under nitrogen, and to this one was added a solution of the above obtained residue in methanol (3.5 mL). The mixture was stirred at room temperature for 20 hours. The resulting mixture was evaporated under reduced pressure. The residue was washed with diisopropyl ether and dried in vacuo to give the target compound (997 mg).

MS ((+)ESI) m/z: 446 (M−HCl+H)⁺.

### EXAMPLE 173-4

### Methyl({2-[((2S)-5-{[(benzyloxy)carbonyl]-amino}-2-{[(1-methyl-1H-indol-2-yl)carbonyl]-amino}pentanoyl)amino]phenyl}thio)acetate

The target compound was obtained in a similar manner to that of Example 131.

MS ((+)ESI) m/z: 625 (M+Na)⁺.

### EXAMPLE 174

### ({2-[((2S)-5-{[(Benzyloxy)carbonyl]amino}-2-{[(1-methyl-1H-indol-2-yl)carbonyl]amino}pentanoyl)-amino]phenyl}thio)acetic acid

The target compound was obtained in a similar manner to that of Example 132.

MS ((−)ESI) m/z: 587 (M−H)⁻.

¹H-NMR (DMSO-d₆): δ 1.45-2.1 (4H, m), 3.0-3.2 (2H, m), 3.65 (2H, s), 3.9 9 (3H, s), 4.55-4.75 (H, m), 5.01 (1H, s), 7.05-7.4 (2H, m), 7.4-7.6 (10H, m), 7.6 6 (1H, d, J=7.8 Hz), 7.76 (1H, d, J=7.9 Hz).

### EXAMPLE 175

### Methyl{[2-({(2S)-5-{[(benzyloxy)carbonyl]amino)-2-[(2-quinolinylcarbonyl)amino]pentanoyl}amino)-phenyl]thio}acetate

The target compound was obtained in a similar manner to that of Example 131.

MS ((+)ESI) m/z: 623 (M+Na)⁺.

### EXAMPLE 176

### {[2-({(2S)-5-{[(Benzyloxy)carbonyl]amino}-2-[(2-quinolinylcarbonyl)amino]pentanoyl}amino)phenyl]-thio}acetic acid

The target compound was obtained in a similar manner to that of Example 132.

MS ((−)ESI) m/z: 585 (M−H)⁻.

¹H-NMR (DMSO-d₆): δ 1.4-1.7 (2H, m), 1.85-2.2 (2H, m), 3.0-3.2 (2H, m), 3.67 (2H, m), 4.75-4.95 (1H, m), 4.99 (2H, s), 7.15-7.95 (11H, m), 8.1-8.25 (3H, m), 8.61 (1H, d, J=8.5 Hz).

### EXAMPLE 177

### Methyl 6-({(2S)-5-{[(benzyloxy)carbonyl]amino}-2-[(1H-indol-3-ylacetyl)amino]pentanoyl}amino)-hexanoate

The target compound was obtained in a similar manner to that of Example 131.

MS ((+)ESI) m/z: 573 (M+Na)⁺.

### EXAMPLE 178

### 6-({(2S)-5-{[(Benzyloxy)carbonyl]amino}-2-[(1H-indol-3-ylacetyl)amino]pentanoyl}amino)hexanoic acid

The target compound was obtained in a similar manner to that of Example 132.

MS ((+)ESI) m/z: 573 (M+Na)⁺.

¹H-NMR (DMSO-d₆): δ 1.1-1.8 (12H, m), 2.17 (2H, t, J=7.3 Hz), 2.85-3.1 (4H, m), 3.56 (2H, d, J=3.2 Hz), 4.1-4.3 (1H, m), 5.00 (2H, s), 6.8.5-7.1 (2H, m), 7.15-7.45 (8H, m).

### EXAMPLE 179

### Methyl 6-[((2S)-5-{((benzyloxy)carbonyl]amino}-2-{[3-(1H-indol-3-yl)propanoyl]amino}pentanoyl)-amino]hexanoate

The target compound was obtained in a similar manner to that of Example 131.

MS ((+)ESI) m/z: 587 (M+Na)⁺.

### EXAMPLE 180

### 6-[((2S)-5-{[(Benzyloxy)carbonyl]amino}-2-{[3-(1H-indol-3-yl)propanoyl]amino}pentanoyl)amino]-hexanoic acid

The target compound was obtained in a similar manner to that of Example 132.

MS ((−)ESI) m/z: 549 (M−H)⁻.

¹H-NMR (DMSO-d₆): δ 1.15-1.7 (10H, m), 2.18 (2H, t, J=7.2 Hz), 2.35-2.6 (2H, m), 2.8-3.1 (6H, m), 4.1-4.3 (1H, m), 5.00 (2H, s), 6.9-7.15 (3H, m), 7.2-7.45 (6H, m), 7.53 (1H, d, J=7.5 Hz).

### EXAMPLE 181

### Methyl 3-[2-({(2S)-5-{[(benzyloxy)carbonyl]amino}-2-[(2,3-dihydro-1-benzofuran-2-ylcarbonyl)amino]-pentanoyl}amino)phenyl]propanoate

A mixture of methyl (2E)-3-[2-[[(2S)-2-[(1-benzofuran-2-ylcarbonyl)amino)-5-[[(benzyloxy)carbonyl]amino]pentanoyl]amino]-phenyl]acrylate (734 mg) and 10% palladium on activated carbon (50% wet, 1.5 g) in a mixture of methanol (20 mL), N,N-dimethylformamide (10 mL) and acetic acid (10 mL) was stirred at 45° C. in the presence of hydrogen at an atmospheric pressure for 1.5 hours. Palladium on activated carbon was removed by filtration through celite and the filtrate was evaporated under reduced pressure.

To the mixture of the residue in a mixture of tetrahydrofuran (80 mL) and water (20 mL), was added benzyloxycarbonyl chloride (242 mg) below 20° C. with adjusting pH to 8.5 with 1N sodium hydroxide. The mixture was stirred at room temperature for 2 hours. The resulting mixture was diluted with ethyl acetate and separated. The organic layer was washed successively with water three times and brine, dried over anhydrous magnesium sulfate, and evaporated under reduced pressure. The residue was purified by column chromatography on silica gel (hexane/ethyl acetate=1:1 to 1:2) to give the target compound (214 mg). Methyl 3-{2-[((2S)-2-[(1-benzofuran-2-ylcarbonyl)amino]-5-{[(benzyloxy)carbonyl]amino}pentanoyl)amino]phenyl}propanoate was also obtained.

MS ((+)ESI) m/z: 596 (M+Na)⁺.

### EXAMPLE 182

### 3-[2-({(2S)-5-{[(Benzyloxy)carbonyl]amino}-2-[(2,3-dihydro-1-benzofuran-2-ylcarbonyl)amino]-pentanoyl}amino)phenyl]propanoic acid

The target compound was obtained in a similar manner to that of Example 132.

MS ((−)ESI) m/z: 558 (M−H)⁻.

¹H-NMR (DMSO-d₆): δ 1.3-1.9 (4H, m), 2.35-2.55 (2H, m), 2.65-2.8 (2H, m), 2.95-3.5 (5H, m), 4.45-4.6 (1H, m), 5.0-5.05 (2H, m), 5.15-5.3 (1H, m), 6.8-6.9 (2H, m), 7.05-7.4 (11H, m).

### EXAMPLE 183-1

### 3-(Tritylamino)-1-propanol

To a solution of 3-amino-1-propanol (3.0 g) and triethylamine (4.45 g) in dichloromethane (30 mL), was added a solution of trityl chloride (11.7 g) in dichloromethane (90 mL) at 5° C. under nitrogen. The mixture was stirred at room temperature for 22 hours.

The resulting mixture was poured into 1N hydrochloric acid and the aqueous layer was extracted with ethyl acetate. The organic layer was washed successively with water, saturated aqueous sodium bicarbonate and brine, dried over anhydrous magnesium sulfate, and evaporated under reduced pressure. The residue was purified by column chromatography on silica gel (hexane/ethyl acetate=5:1 to 2:1) to give the target compound (1.36 g).

MS ((+)ESI) m/z: 340 (M+Na)⁺.

### EXAMPLE 183-2

### 3-(Tritylamino)propyl methanesulfonate

To a solution of 3-(tritylamino)-1-propanol (500 mg) obtained in Example 183-1 in dichloromethane (10 mL), were added triethylamine (0.40 mL) and methanesulfonylchloride (0.165 mL) at 5° C. under nitrogen. The mixture was stirred at the same temperature for 3 hours. The resulting mixture was poured into 1N hydrochloric acid and the aqueous layer was extracted with ethyl acetate. The organic layer was washed successively with water, saturated aqueous sodium bicarbonate and brine, dried over anhydrous magnesium sulfate, evaporated, and dried in vacuo to give the target compound (574 mg).

MS ((+)ESI) m/z: 418 (M+Na)⁺.

### EXAMPLE 183-3

### Methyl {[3-(tritylamino)propyl]thio}acetate

To a solution of methyl mercaptoacetate (163 mg) in N,N-dimethylformamide (13 mL), was added sodium methoxide (159 mg), followed by 3-(tritylamino)propyl methanesulfonate (553 mg) obtained in Example 183-2 and tetrabutylammmonium iodide (568 mg) at room temperature under nitrogen. The mixture was stirred at 50° C. for 30 minutes.

The resulting mixture was poured into water and the aqueous layer was extracted with ethyl acetate. The organic layer was washed successively with water three times and brine, dried over anhydrous magnesium sulfate, evaporated, and dried in vacuo to give the target compound (466 mg).

### EXAMPLE 183-4

### Methyl[(3-aminopropyl)thio]acetate hydrochloride

To a solution of methyl [[3-(tritylamino)propyl]thio]acetate (463 mg) obtained in Example 183-3 in dichloromethane (5 mL), were added anisole (0.62 mL) and trifluoroacetic acid (0.44 mL) at 5° C. under nitrogen. The mixture was stirred at the same temperature for 2.5 hours. The resulting mixture was evaporated under reduced pressure. The residue was washed with isopropyl ether and dissolved into methanol, followed by addition of hydrogen chloride methanol reagent 10, evaporated, and drying in vacuo to give the target compound (234 mg)

MS ((+)ESI) m/z: 164 (M−HCl+H)⁺.

### EXAMPLE 183-5

### Methyl(8S)-8-[(tert-butoxycarbonyl)amino]-3,9-dioxo-1-phenyl-2-oxa-14-thia-4,10-diazahexadecan-16-oate

The target compound was obtained in a similar manner to that of Example 131.

MS ((+) ESI) m/z: 534 (M+Na)⁺.

### EXAMPLE 183-6

### Methyl (8S)-8-amino-3,9-dioxo-1-phenyl-2-oxa-14-thia-4,10-diazahexadecan-16-oate hydrochloride

The target compound was obtained in a similar manner to that of Example 169-4.

MS ((+)ESI) m/z: 434 (M−HCl+Na)⁺.

### EXAMPLE 183-7

### Methyl(8S)-8-[(1-benzofuran-2-ylcarbonyl)amino]-3,9-dioxo-1-phenyl-2-oxa-14-thia-4,10-diazahexadecan-16-oate

The target compound was obtained in a similar manner to that of Example 131.

MS ((+)ESI) m/z: 578 (M+Na)⁺.

### EXAMPLE 184

### Sodium (8S)-8-[(1-benzofuran-2-ylcarbonyl)-amino]-3,9-dioxo-1-phenyl-2-oxa-14-thia-4,10-diazahexadecan-16-oate

To a solution of methyl (8S)-8-[(1-benzofuran-2-ylcarbonyl)amino]-3,9-dioxo-1-phenyl-2-oxa-14-thia-4,10-diazahexadecan-16-oate (63 mg) in 1,4-dioxane (3 mL), was added 1N sodium hydroxide (0.34 mL) at room temperature. The mixture was stirred at 45° C. for 4.5 hours. The resulting mixture was poured into 1N hydrochloric acid, and the aqueous layer was extracted with a mixture of chloroform and methanol (5:1). The organic layer was dried over anhydrous magnesium sulfate, evaporated, and dried in vacuo to give the acid product. The residue was dissolved into methanol, added 1N sodium hydroxide (0.12 mL), evaporated, and dried in vacuo to give the target compound (64 mg).

MS ((+)ESI) m/z: 586 (M+Na)⁺.

¹H-NMR (DMSO-d₆): δ 1.35-1.9 (6H, m), 2.45-2.6 (2H, m), 2.91 (2H, s), 2.95-3.25 (4H, m), 4.35-4.5 (1H, m), 4.99 (2.H, s), 7.25-7.8 5 (10H m).

### EXAMPLE 185-1

### Ethyl 6-[(tert-butoxycarbonyl)amino]hexanoate

To a suspension of ethyl 6-aminohexanoate hydrochloride (1.5 g) in tetrahydrofuran (20 mL), were added triethylamine (853 mg) and di-tert-butyl dicarbonate (1.84 g) at 5° C. under nitrogen. The mixture was stirred at the same temperature for 40 minutes.

The resulting mixture was poured into 1N hydrochloric acid and the aqueous layer was extracted with ethyl acetate. The organic layer was washed successively with saturated aqueous sodium bicarbonate and brine, dried over anhydrous magnesium sulfate, and evaporated under reduced pressure. The residue was purified by column chromatography on silica gel (hexane/ethyl acetate=5:1 to 3:1) to give the target compound (1.94 g).

MS ((+)ESI) m/z: 282 (M+Na)⁺.

### EXAMPLE 185-2

### Ethyl 6-[(tert-butoxycarbonyl)(methyl)amino]-hexanoate

To a suspension of sodium hydride (60% in oil, 85 mg) in N,N-dimethylformamide (6 mL), was added a solution of ethyl 6-[(tert-butoxycarbonyl)amino]-hexanoate (500 mg) obtained in Example 185-1 in N,N-dimethylformamide (2 mL) at 5° C. under nitrogen. The mixture was stirred at the same temperature for 1 hour and at room temperature for 20 minutes. To this one was added iodomethane (301 mg) at 5° C., and the mixture was stirred at room temperature for 3 days.

The resulting mixture was poured into water, and the aqueous layer was extracted with a mixture of hexane and ethyl acetate (1:1). The organic layer was washed successively with water two times and brine, dried over anhydrous magnesium sulfate, and evaporated under reduced pressure. The residue was purified by column chromatography on silica gel (hexane/ethyl acetate=10:1 to 5:1) to give the target compound (222 mg)

MS ((+)ESI) m/z: 296 (M+Na)⁺.

### EXAMPLE 185-3

### Ethyl 6-(methylamino)hexanoate hydrochloride

The target compound was obtained in a similar manner to that of Example 169-4.

MS ((+)ESI) m/z: 174 (M−HCl+H)⁺.

### EXAMPLE 185-4

### Ethyl 6-[{(2S)-5-{[(benzyloxy)carbonyl]amino}-2-[(tert-butoxycarbonyl)amino}pentanoyl}(methyl)-amino]hexanoate

The target compound was obtained in a similar manner to that of Example 131.

MS ((+)ESI) m/z: 544 (M+Na)⁺.

### EXAMPLE 185-5

### Ethyl 6-[((2S)-2-amino-5-{[(benzyloxy)carbonyl]-amino}pentanoyl)(methyl)amino]hexanoate hydrochloride

The target compound was obtained in a similar manner to that of Example 169-4.

MS ((+)ESI) m/z: 422 (M−HCl+H)⁺.

### EXAMPLE 185-6

### Ethyl 6-[((2S)-2-[(1-benzofuran-2-ylcarbonyl)-amino]-5-{[(benzyloxy)carbonyl]amino}pentanoyl)-(methyl)amino]hexanoate

The target compound was obtained in a similar manner to that of Example 131.

MS ((+)ESI) m/z: 588 (M+Na)⁺.

### EXAMPLE 186

### Sodium 6-[((2S)-2-[(1-benzofuran-2-ylcarbonyl)-amino]-5-{[(benzyloxy)carbonyl]amino}pentanoyl)-(methyl)amino]hexanoate

The target compound was obtained in a similar manner to that of Example 132.

MS ((−) ESI) m/z: 536 (M−Na)⁻.

¹H-NMR (DMSO-d₆): δ 1.1-1.95 (12H, m), 2.8-3.6 (7H, m), 4.8-4.95 (1H, m), 5.00 (2H, s), 7.15-7.85 (10H, m).

### EXAMPLE 187-1

### 9H-Fluoren-9-ylmethyl (4S)-4-(3-{[(benzyloxy)-carbonyl]amino}propyl)-5-oxo-1,3-oxazolidine-3-carboxylate

A mixture of (2S)-5-[[(benzyloxy)carbonyl]-amino]-2-[[(9H-fluoren-9-ylmethoxy)carbonyl]-amino]pentanoic acid (2.0 g), paraformaldehyde (1.23 g) and p-toluenesulfonic acid hydrate (78 mg) in toluene (40 mL) was distilled for 40 minutes to remove water as toluene azeotrope.

The resulting mixture was poured into 5% aqueous sodium bicarbonate and the aqueous layer was extracted with ethyl acetate. The organic layer was washed successively with 5% aqueous sodium bicarbonate and brine, dried over anhydrous magnesium sulfate, and evaporated under reduced pressure. The residue was purified by column chromatography on silica gel (chloroform/methanol=20:1 to 10:1) to give a mixture (1.13 g) of the target compound and (4S)-1-[(benzyloxy)carbonyl]-3-[(9H-fluoren-9-ylmethoxy)carbonyl]hexahydro-1H-1,3-diazepine-4-carboxylic acid.

MS ((+)ESI) m/z: 537 (M+Na)⁺.

### EXAMPLE 187-2

### (2S)-5-{[(Benzyloxy)carbonyl]amino}-2-[[(9H-fluoren-9-ylmethoxy)carbonyl](methyl)amino]-pentanoic acid

To a solution of a mixture (400 mg) of 9H-fluoren-9-ylmethyl (4S)-4-[3-[[(benzyloxy)-carbonyl]amino]propyl]-5-oxo-1,3-oxazolidine-3-carboxylate and (4S)-1-[(benzyloxy)carbonyl]-3-[(9H-fluoren-9-ylmethoxy)carbonyl]hexahydro-1H-1,3-diazepine-4-carboxylic acid obtained in Example 187-1 in chloroform (6 mL), were added trifluoroacetic acid (6 mL) and triethylsilane (279 mg) at room temperature under nitrogen. The mixture was stirred at the same temperature for 22 hours.

The resulting mixture was evaporated under reduced pressure. The residue was purified by column chromatography on silica gel (chloroform/methanol=50:1 to 5:1) to give the target compound (381 mg)

MS ((+)ESI) m/z: 652 (M+Na)⁺.

### EXAMPLE 187-3

### Methyl 6-({(2S)-5-{[(benzyloxy)carbonyl]amino}-2-[[(9H-fluoren-9-ylmethoxy)carbonyl](methyl)amino]-pentanoyl}amino)hexanoate

The target compound was obtained in a similar manner to that of Example 131.

MS ((+)ESI) m/z: 652 (M+Na)⁺.

### EXAMPLE 187-4

### Methyl 6-{[(2S)-5-{(benzyloxy)carbonyl]amino}-2-(methylamino)pentanoyl]amino}hexanoate

Piperidine (20% in N,N-dimethylformamide, 4 mL) was added to methyl 6-[[(2S)-5-[[(benzyloxy)-carbonyl]amino]-2-[[(9H-fluoren-9-ylmethoxy)-carbonyl](methyl)amino]pentanoyl]amino]hexanoate (415 mg) obtained in Example 187-3 at room temperature, and the mixture was stirred at the same temperature for 10 minutes. The resulting mixture was evaporated under reduced pressure. The residue was purified by reverse-phase column chromatography to give the target compound (129 mg).

MS ((+)ESI) m/z: 408 (M+H)⁺.

### EXAMPLE 187-5

### Methyl 6-[((2S)-2-[(1-benzofuran-2-ylcarbonyl)-(methyl)amino]-5-{[(benzyloxy)carbonyl]amino}-pentanoyl)amino]hexanoate

The target compound was obtained in a similar manner to that of Example 131.

MS ((+) ESI) m/z: 574 (M+Na)⁺.

### EXAMPLE 188

### Sodium 6-[((2S)-2-[(1-benzofuran-2-ylcarbonyl)-(methyl)amino]-5-{[(benzyloxy)carbonyl]amino}-pentanoyl)amino]hexanoate

To a solution of methyl 6-[[(2S)-2-[(1-benzofuran-2-ylcarbonyl)(methyl)amino]-5-[[(benzyloxy)carbonyl]amino]pentanoyl]amino]-hexanoate (132 mg) in 1,4-dioxane (10 mL), was added 1N sodium hydroxide (0.36 mL) at room temperature. The mixture was stirred at the same temperature for 16 hours.

The resulting mixture was poured into 1N hydrochloric acid and the aqueous layer was extracted with chloroform. The organic layer was dried over anhydrous magnesium sulfate, evaporated under reduced pressure. The residue was purified by column chromatography on silica gel (chloroform/methanol=20:1 to 15:1), followed by treatment of 1N sodium hydroxide to give the target compound (57 mg).

MS ((−)ESI) m/z: 536 (M−Na)⁻.

¹H-NMR (DMSO-d₆): δ 1.1-1.9 (12H, m), 2.9-3.7 (7H, m), 3.85-4.15 (1H, m), 5.01 (2H, s), 7.2-7.5 (8H, m), 7.55-7.8 (2H, m).

### EXAMPLE 189-1

### Methyl(2S)-5-{[(benzyloxy)carbonyl]amino}-2-(tritylamino)pentanoate

To a suspension of methyl (2S)-2-amino-5-[[(benzyloxy)carbonyl]amino]pentanoate hydrochloride (1.0 g) and triethylamine (767 mg) in dichloromethane (20 mL), was added a solution of trityl chloride (968 mg) in dichloromethane (4 mL) at 5° C. under nitrogen. The mixture was stirred at room temperature for 12 hours.

The resulting mixture was poured into 1N hydrochloric acid and the aqueous layer was extracted with ethyl acetate. The organic layer was washed successively with water three times, saturated aqueous sodium bicarbonate and brine, dried over anhydrous magnesium sulfate, and evaporated under reduced pressure. The residue was purified by column chromatography on silica gel (hexane/ethyl acetate=3:1 to 2:1) to give the target compound (1.54 g)

MS ((+)ESI) m/z: 545 (M+Na)⁺.

### EXAMPLE 189-2

### Methyl (2S)-5-[[(benzyloxy)carbonyl](methyl)-amino]-2-(tritylamino)pentanoate

To a suspension of sodium hydride (60% in oil, 42 mg) in N,N-dimethylformamide (10 mL), was added methyl (2S)-5-[[(benzyloxy)carbonyl]amino]-2-(tritylamino)pentanoate (500 mg) obtained in Example 189-1 at 5° C. under nitrogen. The mixture was stirred at the same temperature for 50 minutes.

To this one was added iodomethane (149 mg) at 5° C., and the mixture was stirred at room temperature for 4 hours. The resulting mixture was poured into water and the aqueous layer was extracted with ethyl acetate. The organic layer was washed successively with water two times and brine, dried over anhydrous magnesium sulfate, and evaporated under reduced pressure. The residue was purified by column chromatography on silica gel (hexane/ethyl acetate=5:1 to 3:1) to give the target compound (385 mg).

MS ((+)ESI) m/z: 559 (M+Na)⁺.

### EXAMPLE 189-3

### Methyl(2S)-2-amino-5-[[(benzyloxy)carbonyl]-(methyl)amino]pentanoate hydrochloride

The target compound was obtained in a similar manner to that of Example 183-4.

MS ((+)ESI) m/z: 295 (M−HCl+H)⁺.

### EXAMPLE 189-4

### Methyl(2S)-2-[(1-benzofuran-2-ylcarbonyl)amino]-5-[[(benzyloxy)carbonyl](methyl)amino]pentanoate

The target compound was obtained in a similar manner to that of Example 131.

MS ((+)ESI) m/z: 461 (M+Na)⁺.

### EXAMPLE 189-5

### (2S)-2-[(1-Benzofuran-2-ylcarbonyl)amino]-5-[[(benzyloxy)carbonyl](methyl)amino]pentanoic acid

To a solution of methyl (2S)-2-[(1-benzofuran-2-ylcarbonyl)amino]-5-[[(benzyloxy)-carbonyl](methyl)amino]pentanoate (267 mg) obtained in Example 189-4 in methanol (5 mL), was added 1N sodium hydroxide (1.22 mL) at room temperature. The mixture was stirred at the same temperature for 80 minutes. To this resulting mixture was added 1N hydrochloric acid (1.22 mL), evaporated, and dried in vacuo to give the target compound (339 mg).

MS ((−)ESI) m/z: 423 (M−H)⁻.

### EXAMPLE 189-6

### Methyl 6-({(2S)-2-[(1-benzofuran-2-ylcarbonyl)-amino]-5-[[(benzyloxy)carbonyl](methyl)amino]-pentanoyl}amino)hexanoate

The target compound was obtained in a similar manner to that of Example 131.

MS ((+)ESI) m/z: 574 (M+Na)⁺.

### EXAMPLE 190

### Sodium 6-({(2S)-2-[(1-benzofuran-2-ylcarbonyl)-amino]-5-[[(benzyloxy)carbonyl]-(methyl)amino]-pentanoyl}amino)hexanoate

To a solution of methyl 6-[[(2S)-2-[(1-benzofuran-2-ylcarbonyl)amino]-5-[[(benzyloxy)-carbonyl](methyl)amino]pentanoyl]amino]hexanoate (291 mg) in methanol (5 mL), was added 1N sodium hydroxide (0.61 mL) at room temperature. The mixture was stirred at 45° C. for 130 minutes. The resulting mixture was evaporated and dried in vacuo to give the target compound (270 mg).

MS ((+)ESI) m/z: 560 (M+H)⁺.

¹H-NMR (DMSO-d₆): δ 1.1-1.95 (12H, m), 2.7-3.6 (7H, m), 4.3-4.5 (1H, m), 5.03 (2H, s), 7.15-7.5 (8H, m), 7.55-7.8 (2H, m)

### EXAMPLE 191-1

### Methyl 6-{[(4-nitrophenyl)sulfonyl]amino}hexanoate

To a suspension of methyl 6-aminohexanoate hydrochloride (500 mg) in dichloromethane (15 mL), were added 4-nitrobenzenesulfonyl chloride (640 mg) and triethylamine (585 mg) at 5° C. under nitrogen. The mixture was stirred at 5° C. for 1 hour.

The resulting mixture was poured into 1N hydrochloric acid and the aqueous layer was extracted with ethyl acetate. The organic layer was washed successively with 1N hydrochloric acid, water and brine, dried over anhydrous magnesium sulfate, evaporated, and dried in vacuo to give the target compound (915 mg)

MS ((+)ESI) m/z: 353 (M+Na)⁺.

### EXAMPLE 191-2

### Benzyl{(4S)-4-[(tert-butoxycarbonyl)amino]-5-hydroxypentyl}carbamate

To a solution of methyl 6-[[(4-nitrophenyl)sulfonyl]amino]hexanoate (3.0 g) in tetrahydrofuran (30 mL), were added N-methylmorpholine (828 mg) and ethyl chloroformate (888 mg) at −5° C. under nitrogen. The mixture was stirred at the same temperature for 20 minutes. To this one was added sodiumborohydride (929 mg) followed by methanol (30 mL) dropwise at −5° C. The mixture was stirred at the same temperature for 2 hours.

1N Hydrochloric acid was added to the resulting mixture below 10° C. to adjust pH to 6.5. After concentration under reduced pressure, the residue was poured into 1N hydrochloric acid, and the aqueous layer was extracted with ethyl acetate. The organic layer was washed successively with water, 5% aqueous sodium bicarbonate and water, dried over anhydrous magnesium sulfate, and evaporated under reduced pressure. The residue was purified by column chromatography on silica gel (hexane/ethyl acetate=1:1 to 1:2) to give the target compound (2.45 g).

MS ((+)ESI) m/z: 375 (M+Na)⁺.

### EXAMPLE 191-3

### Methyl 6-{{(2S)-5-{[(benzyloxy)carbonyl]amino}-2-[(tert-butoxycarbonyl)amino]pentyl}[(4-nitrophenyl)sulfonyl]amino}hexanoate

To a solution of methyl 6-[[(4-nitrophenyl)sulfonyl]amino]hexanoate (281 mg) obtained in Example 191-1 and benzyl [(4S)-4-[(tert-butoxycarbonyl)amino]-5-hydroxypentyl]carbamate (450 mg) obtained in Example 191-2 in dichloromethane (10 mL), were added triphenylphosphine (402 mg) and diethyl azodicarboxylate (0.241 mL) at 5° C. under nitrogen. The mixture was stirred at room temperature for 5 hours.

The resulting mixture was poured into 1N hydrochloric acid and the aqueous layer was extracted with ethyl acetate. The organic layer was washed successively with saturated aqueous sodium bicarbonate, water and brine, dried over anhydrous magnesium sulfate, and evaporated under reduced pressure. The residue was purified by column chromatography on silica gel (hexane/ethyl acetate=2:1 to 4:3) to give the target compound (200 mg)

MS ((+)ESI) m/z: 687 (M+Na)⁺.

### EXAMPLE 191-4

### Methyl 6-{((2S)-2-amino-5-{[(benzyloxy)carbonyl]-amino}pentyl)[(4-nitrophenyl)sulfonyl]amino}-hexanoate hydrochloride

The target compound was obtained in a similar manner to that of Example 169-4.

MS ((+)ESI) m/z: 565 (M−HCl+H)⁺.

### EXAMPLE 191-5

### Methyl 6-{((2S)-2-[(1-benzofuran-2-ylcarbonyl)-amino]-5-{[(benzyloxy)carbonyl]amino}pentyl)-[(4-nitrophenyl)sulfonyl]amino}hexanoate

The target compound was obtained in a similar manner to that of Example 131.

MS ((+)ESI) m/z: 731 (M+Na)⁺.

### EXAMPLE 191-6

### Methyl 6-[((2S)-2-[(1-benzofuran-2-ylcarbonyl)-amino]-5-{[(benzyloxy)carbonyl]amino}pentyl)(tert-butoxycarbonyl)amino]hexanoate

To a solution of methyl 6-[[(2S)-2-[(1-benzofuran-2-ylcarbonyl)amino]-5-[[(benzyloxy)-carbonyl]amino]pentyl][(4-nitrophenyl)sulfonyl]-amino]hexanoate (129 mg) obtained in Example 191-5 in N,N-dimethylformamide (2 mL), were added potassium carbonate (76 mg) and benzenethiol (0.037 mL) at room temperature under nitrogen. The mixture was stirred at the same temperature for 15 hours.

To this one was added a solution of di-tert-butyl dicarbonate (99 mg) in tetrahydrofuran (1 mL) at room temperature, and the mixture was stirred at the same temperature for 2 hours. The resulting mixture was poured into 1N hydrochloric acid, and the aqueous layer was extracted with ethyl acetate. The organic layer was washed successively with saturated aqueous sodium bicarbonate, water two times and brine, dried over anhydrous magnesium sulfate, and evaporated under reduced pressure. The residue was purified by column chromatography on silica gel (hexane/ethyl acetate=2:1 to 1:1) to give the target compound (71 mg)

MS ((+)ESI) m/z: 623 (M+Na)⁺.

### EXAMPLE 191-7

### 6-[((2S)-2-[(1-Benzofuran-2-ylcarbonyl)amino]-5-{[(benzyloxy)carbonyl]amino}pentyl)(tert-butoxycarbonyl)amino]hexanoic acid

The target compound was obtained in a similar manner to that of Example 132.

MS ((−)ESI) m/z: 608 (M−H)⁻.

### EXAMPLE 191-8

### 6-[((2S)-2-[(1-Benzofuran-2-ylcarbonyl)amino]-5-{[(benzyloxy)carbonyl]amino}pentyl)amino]hexanoic acid hydrochloride

The target compound was obtained in a similar manner to that of Example 169-4.

MS ((−)ESI) m/z: 508 (M−HCl−H)⁻.

¹H-NMR (DMSO-d₆): δ 1.05-1.7 (10H, m), 2.21 (2H, t, J=7.1 Hz), 2.8-3.2 (6H, m), 4.15-4.41 (H, m), 4.99 (2H, s), 7.15-7.9 (1OH, m).

### EXAMPLE 192-1

### Methyl (2S)-5-{[(benzyloxy)carbonyl]amino}-2-{-(4-nitrophenyl)sulfonyl]amino}pentanoate

To a suspension of methyl (2S)-2-amino-5-[[(benzyloxy)carbonyl]amino]pentanoate hydrochloride (500 mg) in dichloromethane (15 mL), were added 4-nitrobenzenesulfonyl chloride (367 mg) and triethylamine (335 mg) at 5° C. under nitrogen. The mixture was stirred at room temperature for 12 hours. The resulting mixture was poured into 1N hydrochloric acid, and the aqueous layer was extracted with ethyl acetate. The organic layer was washed successively with 1N hydrochloric acid, water two times and brine, dried over anhydrous magnesium sulfate, evaporated, and dried in vacuo to give the target compound (760 mg)

MS ((+)ESI) m/z: 488 (M+Na)⁺.

### EXAMPLE 192-2

### Methyl(2S)-5-{[(benzyloxy)carbonyl]amino}-2-{(4-biphenylylmethyl)[(4-nitrophenyl)sulfonyl]amino}-pentanoate

To a solution of methyl (2S)-5-[[(benzyloxy)carbonyl]amino]-2-[[(4-nitrophenyl)-sulfonyl]amino]pentanoate (744 mg) obtained in Example 192-1 in N,N-dimethylformamide (10 mL), were added potassium carbonate (331 mg) and 4-(bromomethyl)biphenylyl (435 mg) at room temperature under nitrogen. The mixture was stirred at the same temperature for 2.5 hours.

The resulting mixture was poured into water, and the aqueous layer was extracted with ethyl acetate. The organic layer was washed successively with water two times and brine, dried over anhydrous magnesium sulfate, and evaporated under reduced pressure. The residue was purified by column chromatography on silica gel (hexane/ethyl acetate=2:1 to 4:3) to give the target compound (870 mg).

MS ((+)ESI) m/z: 654 (M+Na)⁺.

### EXAMPLE 192-3

### (2S)-5-{[(Benzyloxy)carbonyl]amino}-2-{(4-biphenylylmethyl)[(4-nitrophenyl)sulfonyl]-amino}pentanoic acid

To a solution of methyl (2S)-5-[[(benzyloxy)carbonyl]amino]-2-[(4-biphenylyl-methyl)[(4-nitrophenyl)sulfonyl]amino]pentanoate (856 mg) obtained in Example 192-2 in 1,4-dioxane (5 mL), was added 1N sodium hydroxide (2.78 mL) at room temperature. The mixture was stirred at the same temperature for 12 hours. The resulting mixture was poured into 1N hydrochloric acid, and the aqueous layer was extracted with ethyl acetate. The organic layer was dried over an hydrous magnesium sulfate, evaporated, and dried in vacuo to give the target compound (861 mg)

MS ((−)ESI) m/z: 616 (M−H)⁻.

### EXAMPLE 192-4

### Methyl 6-[((2S)-5-{[(benzyloxy)carbonyl]amino}-2-{(4-biphenylylmethyl)[(4-nitrophenyl)sulfonyl]-amino}pentanoyl)amino]hexanoate

The target compound was obtained in a similar manner to that of Example 131.

MS ((+) ESI) m/z: 767 (M+Na)⁺.

### EXAMPLE 192-5

### Methyl 6-({(2S)-5-{[(benzyloxy)carbonyl]amino}-2-[(4-biphenylylmethyl)amino]pentanoyl}amino)-hexanoate

To a solution of methyl 6-[(2S)-5-[[(benzyloxy)carbonyl]amino]-2-[(4-biphenylylmethyl)[(4-nitrophenyl)sulfonyl]amino]-pentanoyl]amino]hexanoate (415 mg) obtained in Example 192-4 in N,N-dimethylformamide (5 mL), were added potassium carbonate (231 mg) and benzenethiol (123 mg) at room temperature under nitrogen. The mixture was stirred at the same temperature for 12 hours.

The resulting mixture was poured into water, and the aqueous layer was extracted with ethyl acetate. The organic layer was washed successively with water two times and brine, dried over anhydrous magnesium sulfate, and evaporated under reduced pressure. The residue was purified by column chromatography on silica gel (chloroform/methanol=50:1 to 20:1) to give the target compound (206 mg).

MS ((+)ESI) m/z: 560 (M+H)⁺.

### EXAMPLE 193

### Sodium 6-({(2S)-5-{[(benzyloxy)carbonyl]amino}-2-[(4-biphenylylmethyl)amino]pentanoyl}amino)-hexanoate

To a solution of methyl 6-[[(2S)-5-[[(benzyloxy)carbonyl]amino]-2-[(4-biphenylylmethyl)amino]pentanoyl]amino]hexanbate (202 mg) in 1,4-dioxane (3 mL), was added 1N sodium hydroxide (0.54 mL) at room temperature. The mixture was stirred at 55° C. for 1.5 hours. To this resulting mixture was added 1N hydrochloric acid (0.18 mL), evaporated, and dried in vacuo to give the target compound (210 mg)

MS ((+)ESI) m/z: 568 (M+H)⁺.

¹H-NMR (DMSO-d₆): δ 1.15-1.6 (10H, m), 1.84 (2H, t, J=7.0 Hz), 2.2-2.5 (1H, m), 2.85-3.2 (6H, m), 3.4-3.8 (2H, m), 4.99 (2H, s), 7.3-7.8 (14H, m).

### EXAMPLE 194-1

### Methyl 3-{2-[((2S)-5-{[(benzyloxy)carbonyl]-amino}-2-{[(4-nitrophenyl)sulfonyl]amino}-pentanoyl)amino]phenyl}propanoate

The target compound was obtained in a similar manner to that of Example 192-1.

MS ((+)ESI) m/z: 635 (M+Na)⁺.

### EXAMPLE 194-2

### Methyl 3-12-[((2S)-5-{[(benzyloxy)carbonyl]amino}-2-{(4-biphenylylmethyl)[(4-nitrophenyl)sulfonyl]-amino}pentanoyl)amino]phenyl}propanoate

The target compound was obtained in a similar manner to that of Example 192-2.

MS ((+)ESI) m/z: 801 (M+Na)⁺.

### EXAMPLE 194-3

### Methyl 3-[2-({(2S)-5-{[(benzyloxy)carbonyl]amino}-2-[(4-biphenylylmethyl)amino]pentanoyl}amino)-phenyl]propanoate

The target compound was obtained in a similar manner to that of Example 192-5.

MS ((+) ESI) m/z: 594 (M+H)⁺.

### EXAMPLE 195

### 3-[2-({(2S)-5-{[(Benzyloxy)carbonyl]amino}-2-[(4-biphenylylmethyl)amino]pentanoyl}amino)phenyl]-propanoic acid

To a solution of methyl 3-[2-[(2S)-5-[[(benzyloxy)carbonyl]amino]-2-[(4-biphenylyl-methyl)amino]pentanoyl]amino]phenyl]propanoate (90 mg) in 1,4-dioxane (3 mL), was added 1N sodium hydroxide (0.36 mL) at room temperature. The mixture was stirred at 45° C. for 8.5 hours. To this resulting mixture was added 1N hydrochloric acid (0.36 mL), and the mixture was stirred at room temperature for 3.5 hours. The precipitates were collected, washed with a mixture of 1,4-dioxane and water (3:1), and dried in vacuo to give the target compound (68 mg).

MS ((−)ESI) m/z: 578 (M−H)⁻.

¹H-NMR (DMSO-d₆): δ 1.4-1.75 (4H, m), 2.4-2.6 (2H, m), 2.75-2.9 (2H, m), 2.9-3.3 (3H, m), 3.6-4.9 (2H, m), 5.00 (2H, s), 7.1-7.55 (14H, m), 7.55-7.7 (4H, m).

### EXAMPLE 196-1

### Methyl 3-[2-({(2S)-5-{[(benzyloxy) carbonyl]amino}-2-[[(4-nitrophenyl)sulfonyl](2-quinolinylmethyl)-amino]pentanoyl}amino)phenyl]propanoate

To a solution of methyl 3-[2-[[(2S)-5-[[(benzyloxy)carbonyl]amino]-2-[[(4-nitrophenyl)-sulfonyl]amino]pentanoyl]amino]phenyl]propanoate (320 mg) in N,N-dimethylformamide (7 mL), were added potassium carbonate (173 mg), potassium iodide (95 mg) and 2-(chloromethyl)quinoline hydrochloride (123 mg) at 5° C. under nitrogen. The mixture was stirred at room temperature for 24 hours.

The resulting mixture was poured into water, and the aqueous layer was extracted with ethyl acetate. The organic layer was washed successively with water two times and brine, dried over anhydrous magnesium sulfate, and evaporated under reduced pressure. The residue was purified by column chromatography on silica gel (chloroform/ethyl acetate=3:1 to 2:1) to give the target compound (197 mg).

MS ((+)ESI) m/z: 776 (M+Na)⁺.

### EXAMPLE 196-2

### Methyl 3-[2-({(2S)-5-{[(benzyloxy)carbonyl]amino}-2-[(2-quinolinylmethyl)amino]pentanoyl}amino)-phenyl]propanoate

The target compound was obtained in a similar manner to that of Example 192-5.

MS ((+)ESI) m/z: 569 (M+H)⁺.

### EXAMPLE 197

### 3-[2-({(2S)-5-{[(Benzyloxy)carbonyl]amino}-2-[(2-quinolinylmethyl)amino]pentanoyl}amino)-phenyl]propanoic acid

To a solution of methyl 3-[2-[[(2S)-5-[[(benzyloxy)carbonyl]amino]-2-[(2-quinolinylmethyl)amino]pentanoyl]amino]phenyl]propanoate (97 mg) in 1,4-dioxane (3 mL), was added 1N sodium hydroxide (0.43 mL) at room temperature. The mixture was stirred at 45° C. for 6 hours. To this resulting mixture was added 1N hydrochloric acid (0.43 mL), and the mixture was evaporated under reduced pressure. To the residue was added a mixture of chloroform and methanol (5:1), and the insoluble materials were removed by filtration. The filtrate was evaporated and dried in vacuo to give the target compound (97 mg)

MS ((+)ESI) m/z: 555 (M+H)⁺.

¹H-NMR (DMSO-d₆): δ 1.45-1.8 (4H, m), 2.45-2.6 (2H, m), 2.75-2.9 (2H, m), 2.95-3.3 (3H, m), 3.9-4.2 (2H, m), 5.00 (2H, s), 7.1-7.8 (12H, m), 7.9-8.0 (2H, m), 8.25-8.35 (1H, m).

### EXAMPLE 198

### Methyl 4-[2-({(2S)-2,5-bis[(1-benzofuran-2-yl-carbonyl)amino]pentanoyl}amino)ethyl]benzoate

The target compound was obtained in a similar manner to that of Example 131.

MS ((+)ESI) m/z: 604 (M+Na)⁺.

### EXAMPLE 199

### 4-[2-({(2S)-2,5-Bis[(1-benzofuran-2-ylcarbonyl)-amino]pentanoyl}amino)ethyl]benzoic acid

The target compound was obtained in a similar manner to that of Example 132.

MS ((−)ESI) m/z: 566 (M−H)⁻.

¹H-NMR (DMSO-d₆): δ 1.4-1.85 (4H, m), 2.7-2.9 (2H, m), 3.15-3.5 (4H, m), 4.3-4.6 (1H, m), 7.25-7.9 (11H, m), 8.1-8.2 5 (1H, m), 8.5 6 (1H, d, J=8.1 Hz), 8.65-8.8 (1H, m).

### EXAMPLE 200-1

### Methyl 6-({(2S)-5-{[(benzyloxy)carbonyl]amino}-2-[(tert-butoxycarbonyl)amino]pentanoyl}amino)-hexanoate

To a solution of (2S)-5-{[(benzyloxy)-carbonyl]amino}-2-[(tert-butoxycarbonyl)amino]-pentanoic acid (15 g) in N,N-dimethylformamide (150 mL) were added successively 1-hydroxybenzotriazole (8.18 g), 1-(3-dimethylaminopropyl)-3-ethyl-carbodiimide (8.3 g). The mixture was stirred at room temperature for 2 hours. The mixture was quenched by the addition of water (300 mL), and extracted with ethyl acetate (300 mL) The extract was washed successively with water, saturated aqueous sodium hydrogencarbonate and brine (120 mL), and dried over magnesium sulfate. Filtration followed by evaporation gave the target compound (18.9 g) as a white solid.

MS ((+)ESI) m/z: 516 (M+Na)⁺.

### EXAMPLE 200-2

### Methyl 6-[((2S)-2-amino-5-{[(benzyloxy)carbonyl]-amino}pentanoyl)amino]hexanoate hydrochloride

To a suspension of methyl 6-({(2S)-5-{[(benzyloxy)carbonyl]amino}-2-[(tert-butoxycarbonyl)amino]pentanoyl}amino)hexanoate (15 g) obtained in Example 200-1 in 1,4-dioxane (100 mL) was added 4N hydrogen chloride in 1,4-dioxane (150 mL) The mixture was stirred at room temperature for 3 hours. The solvent was removed by evaporation to give the target compound (13 g) as a white solid.

MS ((+)ESI) m/z: 394 (M−HCl+Na)⁺.

### EXAMPLE 200-3

### Methyl 6-[((2S)-2-(benzoylamino)-5-{[(benzyloxy)-carbonyl]amino}pentanoyl)amino]hexanoate

The target compound was obtained in a similar manner to that of Example 27-3.

MS ((+)ESI) m/z: 520 (M+Na)⁺.

### EXAMPLE 201

### 6-[((2S)-2-(Benzoylamino)-5-{[(benzyloxy)-carbonyl]amino}pentanoyl)amino]hexanoic acid

The target compound was obtained in a similar manner to that of Example 28.

MS ((−)ESI) m/z: 482 (M−H)⁻.

¹H-NMR (200 MHz, DMSO-d₆): δ 1.17-1.51 (8H, m), 1.64-1.70 (2H, m), 2.18 (2H, t, J=7.2 Hz), 4.32-4.43 (1H, m), 4.99 (2H, s), 7.23-7.35 (6H, m), 7.41-7.54 (3H, m) 7.86-7.96 (3H, m), 8.36-8.40 (1H, m).

### EXAMPLE 202

### Methyl 6-({(2S)-5-{[(benzyloxy)carbonyl]amino}-2-[(2,2-dimethylpropanoyl)amino]pentanoyl}amino)-hexanoate

The target compound was obtained in a similar manner to that of Example 27-3.

MS ((+)ESI) m/z: 500 (M+Na)⁺.

### EXAMPLE 203

### Sodium 6-({(2S)-5-{[(benzyloxy)carbonyl]amino}-2-[(2,2-dimethylpropanoyl)amino]pentanoyl}amino)-hexanoate

The target compound was obtained in a similar manner to that of Example 41.

MS ((−)ESI) m/z: 462 (M−Na)⁻.

¹H-NMR (200 MHz, DMSO-d₆): δ 1.10 (9H, s), 1.20-1.66 (10H, m), 1.87-1.95 (2H, m), 2.92-3.04 (4H, m), 4.14-4.25 (1H, m), 4.99 (2H, s), 7.28-7.54 (7H, m), 8.01-8.04 (1H, m).

### EXAMPLE 204

### Methyl 6-({(2S)-5-{[(benzyloxy)carbonyl]amino}-2-[(2-pyridinylcarbonyl)amino]pentanoyl}amino)-hexanoate

The target compound was obtained in a similar manner to that of Example 27-3.

MS ((+) ESI) m/z: 521 (M+Na)⁺.

### EXAMPLE 205

### Sodium 6-({(2S)-5-[(benzyloxy)carbonyl]amino}-2-[(2-pyridinylcarbonyl)amino]pentanoyl}amino)-hexanoate

The target compound was obtained in a similar manner to that of Example 41.

MS ((−)ESI) m/z: 483 (M−Na)⁻.

¹H-NMR (200 MHz, DMSO-d₆): δ 1.22-1.44 (8H, m), 1.57-1.79 (2H, m), 1.95-2.02 (2H, m), 2.98-3.04 (4H, m), 4.44-4.55 (1H, m), 4.99 (1H, s), 7.32-7.66 (7H, m), 7.97-8.0 7 (2H, m), 8.2 4-8.33 (1H, m), 8.61-8.6 8 (2H, m).

### EXAMPLE 206

### Methyl 6-{[(2S)-5-{[(benzyloxy)carbonyl]amino}-2-(2-naphthoylamino)pentanoyl]amino}hexanoate

The target compound was obtained in a similar manner to that of Example 27-3.

MS ((+)ESI) m/z: 570 (M+Na)⁺.

### EXAMPLE 207

### Sodium 6-{[(2S)-5-{[(benzyloxy)carbonyl]amino}-2-(2-naphthoylamino)pentanoyl]amino}hexanoate

The target compound was obtained in a similar manner to that of Example 41.

MS ((−)ESI) m/z: 596 (M−Na)⁻.

¹H-NMR (200 MHz, DMSO-d₆): δ 1.23-1.47 (10H, m), 1.85-1.92 (2H, m), 3.01-3.07 (4H, m), 4.42-4.53 (1H, m), 4.99 (2H, s), 7.27-7.63 (8H, m) 7.94-8.06 (4H, m), 8.42-8.47 (1H, m), 8.65 (1H, s), 9.12-9.16 (1H, s).

### EXAMPLE 208

### Methyl 6-({(2S)-5-{[(benzyloxy)carbonyl]amino}-2-[(4-biphenylylcarbonyl)amino]pentanoyl}amino)-hexanoate

The target compound was obtained in a similar manner to that of Example 27-3.

MS ((+)ESI) m/z: 596 (M+Na)⁺.

### EXAMPLE 209

### 6-({(2S)-5-{[(Benzyloxy)carbonyl]amino}-2-[(4-biphenylylcarbonyl)amino]pentanoyl}amino)hexanoic acid

The target compound was obtained in a similar manner to that of Example 28.

MS ((−)ESI) m/z: 558 (M−H)⁻.

¹H-NMR (200 MHz, DMSO-d₆): δ 1.20-1.56 (8H, m), 1.71-1.73 (2H, m), 2.18 (2H, t, J=7.2 Hz), 3.06 (4H, m), 4.35-4.45 (1H, m), 5.00 (2H, s), 7.28-7.54 (8H, m), 7.72-7.79 (5H, m), 7.92-8.02 (3H, m), 8.43-8.47 (1H, m).

### EXAMPLE 210

### Methyl 6-[((2S)-5-{[(benzyloxy)carbonyl]amino}-2-{[(2E)-3-phenyl-2-propenoyl]amino}pentanoyl)-amino]hexanoate

The target compound was obtained in a similar manner to that of Example 27-3.

MS ((+)ESI) m/z: 546 (M+Na)⁺.

### EXAMPLE 211

### Sodium 6-[((2S)-5-{[(benzyloxy)carbonyl]amino}-2-{[(2E)-3-phenyl-2-propenoyl]amino}pentanoyl)-amino]hexanoate

The target compound was obtained in a similar manner to that of Example 41.

MS ((−)ESI) m/z: 509 (M−Na)⁻.

¹H-NMR (200 MHz, DMSO-d₆): δ 1.22-1.65 (10H, m), 1.84-1.91 (2H, m), 2.97-3.04 (4H, m), 4.30-4.37 (1H, m), 4.99 (2H, s), 6.92 (1H, d, J=15.8 Hz), 7.33-7.59 (15H, m), 8.33-8.36 (1H, m), 8.80-8.84 (1H, m).

### EXAMPLE 212

### Methyl 6-[((2S)-S-{[(benzyloxy)carbonyl]amino}-2-{[(2E)-3-(3-pyridinyl)-2-propenoyl]amino}-pentanoyl)amino]hexanoate

The target compound was obtained in a similar manner to that of Example 27-3.

MS ((+)ESI) m/z: 547 (M+Na)⁺.

### EXAMPLE 213

### Sodium 6-[((2S)-5-{[(benzyloxy)carbonyl]amino}-2-{[(2E)-3-(3-pyridinyl)-2-propenoyl]amino}-pentanoyl)amino]hexanoate

The target compound was obtained in a similar manner to that of Example 41.

MS ((−)ESI) m/z: 509 (M−H)⁻.

¹H-NMR (200 MHz, DMSO-d₆): δ 1.24-1.64 (10H, m), 2.18 (2H, t, J=7.2 Hz), 2.98-3.11 (4H, m), 4.30-4.41 (1H, m), 5.00 (2H, s), 6.91 (1H, d, J=15.9 Hz) 7.26-7.51 (8H, m), 7.98-8.07 (2H, m), 8.28-8.32 (1H, m), 8.55-8.56 (1H, m), 8.76-8.77 (1H, m).

### EXAMPLE 214

### Methyl 6-[((2S)-2-[(1-benzothien-2-ylcarbonyl)-amino]-5-{[(benzyloxy)carbonyl]amino}pentanoyl)-amino]hexanoate

The target compound was obtained in a similar manner to that of Example 27-3.

MS ((+)ESI) m/z: 576 (M+Na)⁺.

### EXAMPLE 215

### 6-[((2S)-2-[(1-Benzothien-2-ylcarbonyl)amino]-5-{[(benzyloxy)carbonyl]amino}pentanoyl)amino]-hexanoic acid

The target compound was obtained in a similar manner to that of Example 28.

MS ((−)ESI) m/z: 538 (M−H)⁻.

¹H-NMR (200 MHz, DMSO-d₆): δ 1.17-1.72 (10H, m), 2.18 (2H, t, J=7.2 Hz), 3.01-3.07 (4H, m), 4.32-4.42 (1H, m), 5.00 (2H, s), 7.28-7.50 (8H, m), 7.92-8.06 (3H, m), 8.26 (1H, s), 8.72-8.76 (1H, m), 11.9 (1H, s).

### EXAMPLE 216

### Methyl 6-[((2S)-2-[(1H-benzimidazol-2-ylcarbonyl)-amino]-5-{[(benzyloxy)carbonyl]amino}pentanoyl)-amino]hexanoate

The target compound was obtained in a similar manner to that of Example 27-3.

MS ((+)ESI) m/z: 560 (M+Na)⁺.

### EXAMPLE 217

### 6-[((2S)-2-(1H-Benzimidazol-2-ylcarbonyl)amino]-5-{[(benzyloxy)carbonyl]amino}pentanoyl)amino]-hexanoic acid

The target compound was obtained in a similar manner to that of Example 28.

MS ((−)ESI) m/z: 522 (M−H)⁻.

¹H-NMR (200 MHz, DMSO-d₆): δ 1.17-1.53 (8H, m), 1.74-1.77 (2H, m), 2.19 (2H, t, J=7.2 Hz), 3.00-3.08 (4H, m), 4.41-4.51 (1H, m), 4.99 (2H, s), 7.30-7.35 (7H, m), 7.64-7.70 (2H, m), 8.09-8.14 (1H, m) 8.56-8.61 (1H, m).

### EXAMPLE 218

### Methyl 6-({(2S)-5-{[(benzyloxy)carbonyl]amino}-2-[(cyclopropylacetyl)amino]pentanoyl}amino)-hexanoate

The target compound was obtained in a similar manner to that of Example 27-3.

MS ((+)ESI) m/z: 498 (M+Na)⁺.

### EXAMPLE 219

### Sodium 6-({(2S)-5-{[(benzyloxy)carbonyl]amino}-2-[(cyclopropylacetyl)amino]pentanoyl}amino)-hexanoate

The target compound was obtained in a similar manner to that of Example 41.

MS ((−)ESI) m/z: 460 (M−Na)⁻.

¹H-NMR (200 MHz, DMSO-d₆): δ 0.08-0.14 (2H, m), 0.35-0.43 (2H, m), 0.93 (1H, m), 1.20-1.55 (10H, m), 1.82-1.89 (2H, m), 2.01-2.04 (2H, m), 2.95-2.98 (4H, m), 4.18-4.21 (1H, m), 4.99 (2H, s), 7.21-7.47 (6H, m), 8.06-8.10 (2H, m).

### EXAMPLE 220

### Methyl 6-({(2S)-5-{[(benzyloxy)carbonyl]amino}-2-[(cyclopentylcarbonyl)amino]pentanoyl}amino)-hexanoate

The target compound was obtained in a similar manner to that of Example 27-3.

MS ((+)ESI) m/z: 512 (M+Na)⁺.

### EXAMPLE 221

### 6-({(2S)-5-{(Benzyloxy)carbonyl]amino}-2-[(cyclopentylcarbonyl)amino]pentanoyl}amino)-hexanoic acid

The target compound was obtained in a similar manner to that of Example 28.

MS ((−)ESI) m/z: 474 (M−H)⁻.

¹H-NMR (200 MHz, DMSO-d₆): δ 1.18-1.72 (18H, m), 2.14-2.21 (2H, m), 2.50-2.51 (1H, m), 2.95-3.03 (4H, m), 4.12-4.19 (1H, m), 5.00 (2H, s), 7.25-8.00 (8H, m), 12.5 (1H, br).

### EXAMPLE 222

### Methyl 6-({(2S)-5-{[(benzyloxy)carbonyl]amino}-2-[(1H-pyrrol-2-ylcarbonyl)amino]pentanoyl}amino)-hexanoate

The target compound was obtained in a similar manner to that of Example 27-3.

MS ((+)ESI) m/z: 509 (M+Na)⁺.

### EXAMPLE 223

### Sodium 6-({(2S)-5-{[(benzyloxy)carbonyl]amino}-2-[(1H-pyrrol-2-ylcarbonyl)amino]pentanoyl}amino)-hexanoate

The target compound was obtained in a similar manner to that of Example 41.

MS ((−)ESI) m/z: 471 (M−Na)⁻.

¹H-NMR (200 MHz, DMSO-d₆): δ 1.13-1.75 (10H, m), 11.98-2.05 (2H, m), 2.97-3.06 (4H, m), 4.31-4.38 (1H, m), 4.99 (2H, s), 6.06 (1H, m), 6.83-6.84 (2H, m), 7.33-7.47 (6H, m), 8.10 (1H, m), 8.44-8.49 (1H, m).

### EXAMPLE 224

### Methyl 6-[((2S)-5-{[(benzyloxy)carbonyl]amino}-2-{[(1-methyl-1H-indol-2-yl)carbonyl]amino}-pentanoyl)amino]hexanoate

The target compound was obtained in a similar manner to that of Example 27-3.

MS ((+)ESI) m/z: 573 (M+Na)⁺.

### EXAMPLE 225

### 6-[((2S)-5-{[(Benzyloxy)carbonyl]amino}-2-{[(1-methyl-1H-indol-2-yl)carbonyl]amino}-pentanoyl)amino]hexanoic acid

The target compound was obtained in a similar manner to that of Example 28.

MS ((−)ESI) m/z: 535 (M−H)⁻.

¹H-NMR (200 MHz, DMSO-d₆): δ 1.26-1.49 (8H, m), 1.72 (2H, m), 2.15-2.22 (2H, m), 3.02-3.05 (4H, m), 3.96 (3H, s), 4.36-4.38 (1H, m), 5.00 (2H, s) 6.93-7.74 (10H, m), 7.95-8.01 (2H, m), 8.38-8.42 (1H, m), 12.6 (1H, br).

### EXAMPLE 226

### Methyl 3-[2-({(2S)-5-{[(benzyloxy) carbonyl]amino}-2-[(1H-indol-2-ylcarbonyl)amino]pentanoyl}amino)-phenyl]propanoate

The target compound was obtained in a similar manner to that of Example 27-3.

MS ((+)ESI) m/z: 593 (M+Na)⁺.

### EXAMPLE 227

### 3-[2-({(2S)-5-{[(Benzyloxy)carbonyl]amino}-2-[(1H-indol-2-ylcarbonyl)amino]pentanoyl}amino)phenyl]-propanoic acid

The target compound was obtained in a similar manner to that of Example 28.

MS ((−)ESI) m/z: 555 (M−H)⁻.

¹H-NMR (200 MHz, DMSO-d₆): δ 1.59-1.62 (2H, m), 1.81-1.91 (2H, m), 2.46-2.53 (2H, m), 2.79-2.86 (2H, m), 3.07-3.10 (2H, m), 4.63-4.74 (1H, m), 5.00 (2H, s), 7.07-7.64 (14H, m), 8.57-8.61 (1H, m), 9.58 (1H, br-s), 11.6 (1H, br-s), 12.1 (1H, br-s).

### EXAMPLE 228

### Methyl 3-[2-({(2S)-5-{[(benzyloxy)carbonyl]amino}-2-[(4-biphenylylcarbonyl)amino]pentanoyl}amino)-phenyl]propanoate

The target compound was obtained in a similar manner to that of Example 27-3.

MS ((+)ESI) m/z: 630 (M+Na)⁺.

### EXAMPLE 229

### 3-[2-({(2S)-5-{(Benzyloxy)carbonyl]amino}-2-(4-biphenylylcarbonyl)amino]pentanoyl}amino)phenyl]-propanoic acid

The target compound was obtained in a similar manner to that of Example 28.

MS ((−)ESI) m/z: 592 (M−H)⁻.

¹H-NMR (200 MHz, DMSO-d₆): δ 1.58-1.61 (2H, m), 1.88 (2H, m), 2.45-2.51 (2H, m), 2.78-2.85 (2H, m), 3.06-3.09 (2H, m), 4.59-4.69 (1H, m), 5.01 (2H, s), 7.15-7.53 (13H, m), 7.72-7.8 0 (4H, m), 8.03 (2H, d, J=8.3 Hz), 8.64-8.68 (1H, m), 9.55 (1H, s), 12.1 (1H, br-s).

### EXAMPLE 230

### Methyl 3-[2-({(2 )-5-[(benzyloxy) carbonyl]amino}-2-[(6-quinolinylcarbonyl)amino]pentanoyl}amino)-phenyl]propanoate

The target compound was obtained in a similar manner to that of Example 27-3.

MS ((+)ESI) m/z: 605 (M+Na)⁺.

### EXAMPLE 231

### 3-[2-({(2S)-5-{[(Benzyloxy) carbonyl]amino}-2-[(6-quinolinylcarbonyl)amino]pentanoyl}amino)phenyl]-propanoic acid

The target compound was obtained in a similar manner to that of Example 28.

MS ((−)ESI) m/z: 567 (M−H)⁻.

¹H-NMR (200 MHz, DMSO-d₆): δ 1.51-1.64 (2H, m), 1.81-1.92 (2H, m),2.48-2.52 (2H, m), 2.79-2.86 (2H, m), 3.08-3.11 (2H, m), 4.64-4.76 (1H, m), 5.00 (2H, s), 7.13-7.35 (10H, m), 7.79-7.86 (1H, m), 8.19-8.35 (2H, m), 8.73-8.80 (2H, m), 8.96-8.99 (1H, m), 9.13-9.16 (1H, m), 9.63 (1H, s).

### EXAMPLE 232

### 3-{2-[((2S)-5-{[(Benzyloxy)carbonyl]amino}-2-{[(2-naphthyloxy)carbonyl]amino}pentanoyl)amino]-phenyl}propanoic acid

To a solution of methyl 3-{2-((2S)-2-amino-5-{[(benzyloxy)carbonyl]amino}pentanoyl)-amino]phenyl}propanoate hydrochloride (100 mg) in tetrahydofuran (1 mL), was added 1N sodium hydoxide (0.65 mL). The solution was stirred at room temperature for 1 hour. To the solution was added 2-naphthyl chloridocarbonate (49 mg) at 4° C. The mixture was stirred at room temperature over night.

To the mixture was added water and the mixture was extracted with ethyl acetate. The extract was washed with brine, filtrated, and dried over magnesium sulfate. After concentration under reduced pressure, the residue was purified by column chromatography on silica gel with chloroform and methanol to give the target compound as a white solid.

MS ((+)ESI) m/z: 606 (M+Na)⁺.

¹H-NMR (200 MHz, DMSO-d₆): δ 1.64-1.84 (6H, m), 2.77-2.84 (2H, m), 3.07-3.09 (2H, m), 4.28 (1H, m), 5.02 (1H, s), 7.17-7.36 (11H, m), 7.47-7.65 (3H, m), 7.88-7.95 (3H, m), 8.17-8.21 (1H, m), 9.59 (1H, br-s), 12.1 (1H, br-s).

### EXAMPLE 233-1

### Methyl (2S)-2-[(1-benzothien-2-ylcarbonyl)amino]-5-{(benzyloxy) carbonyl]amino }pentanoate

The target compound was obtained in a similar manner to that of Example 27-3.

MS ((+)ESI) m/z: 463 (M+Na)⁺.

### EXAMPLE 233-2

### (2S)-2-[(1-Benzothien-2-ylcarbonyl)amino]-5-{[(benzyloxy)carbonyl]amino}pentanoic acid

The target compound was obtained in a similar manner to that of Example 28.

MS ((−)ESI) m/z: 425 (M−H)⁻.

### EXAMPLE 233-3

### Methyl 4-{2-[((2S)-2-[(1-benzothien-2-ylcarbonyl)-amino]-5-{[(benzyloxy)carbonyl]amino}pentanoyl)-amino]ethyl}benzoate

The target compound was obtained in a similar manner to that of Example 27-3.

MS ((+)ESI) m/z: 610 (M+Na)⁺.

### EXAMPLE 234

### 4-{2-[((2S)-2-[(1-Benzothien-2-ylcarbonyl)amino]-5-{[(benzyloxy)carbonyl]amino}pentanoyl)amino]-ethyl}benzoic acid

The target compound was obtained in a similar manner to that of Example 28.

MS ((−)ESI) m/z: 572 (M−H)⁻.

¹H-NMR (200 MHz, DMSO-d₆): δ 1.34-1.79 (4H, m), 2.80 (2H, t, J=6.8 Hz), 3.01 (2H, dd, J=6.3, 12.0 Hz), 4.31-4.42 (1H, m), 7.27-7.35 (8H, m), 7.40-7.50 (2H, m), 7.85 (2H, d, J=8.0 Hz), 7.93-8.05 (3H, m), 8.12 (1H, t, J=5.5 Hz), 8.25 (1H, s), 8.74 (1H, d, J=8.0 Hz), 12.80 (1H, br-s).

### EXAMPLE 235

### Methyl(2E)-3-{2-[((2S)-2-[(1-benzothien-2-yl-carbonyl)amino]-5-{[(benzyloxy)carbonyl]amino}-pentanoyl)amino]phenyl}acrylate

The target compound was obtained in a similar manner to that of Example 27-1.

MS ((+)ESI) m/z: 608 (M+Na)⁺.

### EXAMPLE 236

### (2E)-3-{2-[((2S)-2-[(1-Benzothien-2-ylcarbonyl)-amino]-5-{[(benzyloxy)carbonyl}amino}pentanoyl)-amino]phenyl}acrylic acid

The target compound was obtained in a similar manner to that of Example 28.

MS ((−) ESI) m/z: 570 (M−H)⁻.

¹H-NMR (200 MHz, DMSO-d₆): δ 1.51-1.72 (2H, m), 1.79-2.00 (2H, m), 3.03-3.14 (2H, m), 4.63-4.74 (1H, m), 5.01 (2H, s), 6.48 (1H, d, J=15.6 Hz), 7.21-7.49 (11H, m), 7.73-7.83 (2H, m), 7.94-8.05 (2H, m), 8.30 (1H, s), 8.94 (1H, d, J=7.5 Hz), 10.03 (1H, s), 12.39 (1H, br-s).

### EXAMPLE 237

### Methyl 3-{2-[((2S)-2-[(1-benzothien-2-ylcarbonyl)-amino]-5-{[(benzyloxy) carbonyl]amino}pentanoyl)-amino]phenyl}propanoate

The target compound was obtained in a similar manner to that of Example 34-1.

MS ((+) ESI) m/z: 610 (M+Na)⁺.

### EXAMPLE 238

### 3-{2-[(2S)-2-[(1-Benzothien-2-ylcarbonyl)amino]-5-{[( benzyloxy)carbonyl]amino}pentanoyl)amino]-phenyl}propanoic acid

The target compound was obtained in a similar manner to that of Example 28.

MS ((+)ESI) m/z: 596 (M+Na)+.

¹H-NMR (200 MHz, DMSO-d₆): δ 1.51-1.71 (2H, m), 1.78-1.98 (2H, m), 2.44-2.52 (2H, m), 2.82 (2H, t, J=7.0 Hz), 3.03-3.14 (2H, m), 4.58-4.70 (1H, m), 5.01 (2H, s), 7.10-7.36 (10H, m), 7.40-7.51 (2H, m), 7.94-8.05 (2H, m), 8.29 (1H, s), 8.93 (1H, d, J=8.0 Hz), 9.61 (1H, s), 12.15 (1H, br-s).

### EXAMPLE 239

### Methyl 3-(2-{[(2S)-2-[(1-benzothien-2-ylcarbonyl)-amino]-5-({[(2-chlorobenzyl)oxy]carbonyl}amino)-pentanoyl]amino}phenyl)propanoate

The target compound was obtained in a similar manner to that of Example 27-3.

MS ((+)ESI) m/z: 644 (M+Na)⁺.

### EXAMPLE 240

### 3-(2-{[(2S)-2-(1-Benzothien-2-ylcarbonyl)amino]-5-({[(2-chlorobenzyl)oxy]carbonyl}amino)-pentanoyl]amino}phenyl)propanoic acid

The target compound was obtained in a similar manner to that of Example 28.

MS ((−) ESI) m/z: 606 (M−H)⁻.

¹H-NMR (200 MHz, DMSO-d₆): δ 1.50-1.71 (2H, m), 1.80-1.99 (2H, m), 2.43-2.54 (2H, m), 2.82 (2H, t, J=7.5 Hz), 3.05-3.14 (2H, m), 4.59-4.69 (1H, m), 7.12-7.50 (10H, m), 7.94-8.05 (2H, m), 8.29 (1H, s), 8.93 (1H, d, J=7.5 Hz), 9.59 (1H, s), 12.21 (1H, br-s),

### EXAMPLE 241

### Ethyl 4-{2-[((2S)-2-[(1-benzothien-2-ylcarbonyl)-amino]-5-{[(benzyloxy)carbonyl]amino}pentanoyl)-amino]phenyl}butanoate

The target compound was obtained in a similar manner to that of Example 27-1.

MS ((+)ESI) m/z: 638 (M+Na)⁺.

### EXAMPLE 242

### 4-{2-[((2S)-2-[(1-Benzothien-2-ylcarbonyl)amino]-5-{[(benzyloxy)carbonyl]amino}pentanoyl)amino]-phenyl}butanoic acid

The target compound was obtained in a similar manner to that of Example 28.

MS ((+)ESI) m/z: 610 (M+Na)⁺.

¹H-NMR (200 MHz, DMSO-d₆): δ 0.54-1.95 (6H, m), 2.22 (2H, t, J=7.5 Hz), 2.57 (2H, t, J=8.0 Hz), 3.04-3.14 (2H, m), 4.59-4.70 (1H, m), 5.01 (2H, s), 7.13-7.51 (10H, m), 7.94-8.05 (2H, m), 8.30 (1H, s), 8.93 (1H, d, J=7.5 Hz), 9.50 (1H, s), 12.05 (1H, br-s).

### EXAMPLE 243-1

### (2S)-2-(1-Benzofuran-2-ylcarbonyl)amino-5-[(benzyloxycarbonyl)amino]pentanoic acid

To a solution of (2S)-2-amino-5-[(benzyloxycarbonyl)amino]pentanoic acid (5.0 g, 18.77 mmol) in NMP (50 mL), was added BSA (11.6 mL, 46.93 mmol), and the mixture was stirred for 1 hour at room temperature. To the reaction mixture was added a mixture of 1-benzofuran-2-carboxylic acid (3.35 g, 20.65 mmol), PyBOP (10.74 g, 20.65 mmol) and DIEA (7.37 mL, 41.29 mmol) in NMP (40 mL). The mixture was stirred 24 hours at room temperature.

The resultant mixture was partitioned between 25% n-hexane in EtOAc and 10% aqueous KHSO₄ solution. The organic phase was separated, washed with brine, and dried over MgSO₄. Evaporation of the solvent gave a residue, which was purified by column chromatography on silica-gel (CHCl₃—MeOH 9:1) to give the target compound (4.1 g, 49.9%) as a foam.

MS ((−)ESI) m/z: 409 (M−H)⁻.

¹H-NMR (DMSO-d₆): δ 1.40-1.95 (4H, m), 2.95-3.10 (2H, m), 4.30-4.45 (1H, m), 5.01 (2H, s), 7.25-7.45 (7H, m), 7.44-7.53 (1H, m), 7.63-7.82 (3H, m), 8.85 (1H, d, J=7.9 Hz).

### EXAMPLE 243-2

### (2E)-3-{2-[((2S)-2-[(1-Benzofuran-2-ylcarbonyl)-amino]-5-{[(benzyloxy)carbonyl]amino}pentanoyl)-amino]phenyl}acrylic acid

In the 60 mL polypropylene tube with polyethylene flits, to a suspension of wang resin (2.5 g, 0.81 mmole/g), 2-nitrocinnamic acid (782.3 mg, 4.05 mmol), triphenylphpsphine (1.18 g, 4.05 mmol) in THF (20 mL) was added DEAD (637.8 μL, 4.05 mmol). The mixture was shaken for 4 hours at room temperature. After drained the solvent, the resin was washed well with THF and the carboxylic acid loading reaction was repeated. The solvent was drained, washed well subsequently with DMF, MeOH, DCM, Et₂O, and dried under reduced pressure.

To the above resin was added DCM (20 mL), pyridine (6.55 mL, 1.62 mmol) and Ac₂O (3.83 mL, 40.5 mmol). The mixture was shaken overnight at room temperature. After drained the solvent, the resin was washed well subsequently with DMF, MeOH, DCM, Et₂O, and dried under reduced pressure. The resulted resin was treated with 2M SnCl₂—H₂O in DMF (20 mL×2) for 2 hours for the reduction of nitro group. Then, the resin was filtered, washed well subsequently with DMF, MeOH, DCM, Et₂O, and dried under reduced pressure to give 2-aminocinnamic acid loaded wang resin. The obtained resin was divided 2 reaction vessels (2.02 mmol each).

To a suspension of the above 2-aminocinnamic acid loaded wang resin (2.02 mmol), (2S)-2-(1-benzofuran-2-ylcarbonyl) amino-5-[(benzyloxycarbonyl) amino]-pentanoic acid (3.03 mmol) obtained in Example 243-1 and PyBroP (1.42 g, 3.03 mmol) in NMP (15 mL), was added DIEA (1.08 mL, 6.06 mmol). The mixture was shaken for 3 days at room temperature. The solvent was drained, washed well subsequently with DMF, MeOH, DCM, Et₂O, and dried under reduced pressure. After treated with 50% TFA in DCM (20 mL) for 1 hour, the resin was filtered and washed with DCM (15 mL×2). The filtrates were combined, evaporated and purified by HPLC (reverse phase C₁₈, 5 μ, 30 mm×50 mm column, 254 nm, gradient 10-90% 0.05% TFA in CH₃CN/0.05% TFA in H₂O, 40 mL/min.). The fractions containing the target compound were combined, evaporated, and dried under reduced pressure to give the target compound.

MS ((−)ESI) m/z: 554 (M−H)⁻.

¹H-NMR (DMSO-d₆): δ 1.45-2.05 (4H, m), 3.00-3.15 (2H, m), 4.60-4.80 (1H, m), 5.00 (2H, s), 6.48 (1H, d, J=15.8 Hz), 7.2 0-7.55 (12H, m), 7.69 (2H, d, J=9.4 Hz), 7.75-7.85 (2H, m), 8.76 (1H, d, J=7.7 Hz), 10.03 (1H, S), 12.41 (1H, br-s).

### EXAMPLE 244-1

### (2S)-5-(Benzyloxycarbony)amino-2-1[(4-biphenylyl-amino)carbonyl]amino}pentanoic acid

To a solution of (2S)-2-amino-5-[(benzyloxycarbonyl)amino]pentanoic acid (5.0 g, 18.77 mmol) in THF (50 mL), was added BSA (11.6 mL, 46.93 mmol). The mixture was stirred for 1 hour at room temperature. To the reaction mixture was added 4-biphenylyl isocyanate (4.03 g, 20.65 mmol) and the mixture was stirred 24 hours at room temperature. The resultant mixture was partitioned between EtOAc and 10% aqueous KHSO₄ solution. The organic phase was separated, washed with brine, and dried over MgSO₄. Evaporation of the solvent gave a residue, which was purified by column chromatography on silica-gel (CHCl₃—MeOH=9:1) to give the target compound (6.74 g, 73.4%) as a foam.

MS ((−)ESI) m/z: 460 (M−H)⁻.

¹H-NMR (DMSO-d₆): δ 1.40-1.85 (4H, m), 2.95-3.10 (2H, m), 4.10-4.25 (1H, m), 5.01 (2H, s), 6.51 (1H, d, J=7.9 Hz), 7.25-7.65 (5H, m), 8.75 (1H, s), 12.76 (1H, br-s).

### EXAMPLE 244-2

### (2E)-3-{2-[((2S)-5-{[(Benzyloxy)carbonyl]amino}-2-{[(4-biphenylylamino) carbonyl]amino}pentanoyl)-amino]phenyl}acrylic acid

The target compound was obtained from (2S)-5-(benzyloxycarbony)amino-2-{[(4-biphenylylamino)carbonyl]amino}pentanoic acid obtained in Example 244-1 in a similar manner to that of Example 243-2.

MS ((−)ESI) m/z: 605 (M−H)⁻.

¹H-NMR (DMSO-d₆): δ 1.50-1.90 (4H, m), 3.00-3.15 (2H, m), 4.50-4.65 (1H, m), 5.00 (2H, s), 6.48 (1H, d, J=15.8 Hz), 6.56 (1H, d, J=8.2 Hz), 7.25-7.80 (20H, m), 8.81 (1H, s), 10.06 (1H, S), 12.43 (1H, s).

### EXAMPLE 245

### {3-[((2S)-2-[(1-Benzofuran-2-ylcarbonyl)amino]-5-{[(benzyloxy)carbonyl]amino}pentanoyl) amino]-phenyllacetic acid

The target compound was obtained in a similar manner to that of Example 243-1 and 243-2.

MS ((−)ESI) m/z: 542 (M−H)⁻.

¹H-NMR (DMSO-d₆): δ 1.40-1.95 (4H, m), 2.95-3.15 (2H, m), 3.50-3.65 (2H, m), 4.50-4.65 (1H, m), 5.00 (2H, s), 6.96 (1H, d, J=7.6 Hz), 7.20-7.55 (1H, m), 7.65-7.85 (3H, m), 8.75 (1H, d, J=7.7 Hz), 10.15 (1H, S).

### EXAMPLE 246

### {3-[((2S)-5-{[(Benzyloxy)carbonyl]amino}-2-{[(4-biphenylylamino)carbonyl]amino}pentanoyl)amino]-phenyl}acetic acid

The target compound was obtained in a similar manner to that of Example 243-1 and 243-2.

MS ((−)ESI) m/z: 593 (M−H)⁻.

¹H-NMR (DMSO-d6): δ 1.40-1.80 (4H, m), 2.95-3.15 (2H, m), 3.55-3.65 (2H, m), 4.35-4.50 (1H, m), 5.00 (2H, s), 6.54 (1H, d, J=8.2 Hz), 6.96 (1H, d, J=7.5 Hz), 7.20-7.70 (17H, m), 8.80 (1H, s), 10.16 (1H, S).

### EXAMPLE 247

### (2E)-3-{3-[((2S)-2-[(1-Benzofuran-2-ylcarbonyl)-amino]-5-{[(benzyloxy) carbonyl]amino}pentanoyl)-amino]phenyl}acrylic acid

The target compound was obtained in a similar manner to that of Example 243-1 and 243-2.

MS ((−)ESI) m/z: 554 (M−H)⁻.

¹H-NMR (DMSO-d₆): δ 1.40-2.05 (4H, m), 3.00-3.15 (2H, m), 4.50-4.70 (1H, m), 5.00 (2H, s), 6.43 (1H, d, J=15.9 Hz), 7.25-7.90 (16H, m), 7.69 (2H, d, J=9.4 Hz), 8.80 (1H, d, J=7.7 Hz), 10.26 (1H, S).

### EXAMPLE 248

### (2E)-3-{3-[((2S)-5-{[(Benzyloxy) carbonyl]amino}-2-{[(4-biphenylylamino)carbonyl]amino}-pentanoyl)-amino]phenyl}acrylic acid

The target compound was obtained in a similar manner to that of Example 243-1 and 243-2.

MS ((−)ESI) m/z: 605 (M−H)⁻.

¹H-NMR (DMSO-d₆): δ 1.40-1.90 (4H, m), 2.95-3.15 (2H, m), 4.35-4.50 (H, m), 5.00 (2H, s), 6.43 (1H, d, J=15.9 Hz), 6.57 (1H, d, J=8.2 Hz), 7.25-7.70 (19H, m), 7.88 (1H, s), 8.80 (1H, s), 10.28 (1H, S), 12.45 (1H, br-s).

### EXAMPLE 249-1

### 6-[((2S)-5-{[(Benzyloxy)carbonyl]amino}-2-amino}-pentanoyl)amino]hexanoic acid loaded wang resin

In the 60 mL polypropylene tube with polyethylene flits, a suspension of wang resin (3.5 g, 0.81 mmole/g) 6-(9-fluorenylmethoxycarbonylamino)hexanoic acid (3.7 g, 11.4 mmol), MSNT (3.38 g, 11.4 mmol) and NMI (3.62 mL, 45.4 mmol) in DCM (25 mL) was shaken for 2 days at room temperature. The solvent was drained, washed well subsequently with DMF, MeOH, DCM, Et₂O, and dried under reduced pressure. To the above resin was added DCM (25 mL), pyridine (9.19 mL, 113.6 mmol) and Ac₂O (5.37 mL, 56.8 mmol). The mixture was shaken overnight at room temperature. After drained the solvent, the resin was washed well subsequently with DMF, MeOH, DCM, Et₂O, and dried under reduced pressure.

The resulted resin was treated with 20% piperidine in DMF (25 mL×2) for 1 hour to remove Fmoc group. Then, the solvent was drained, washed well subsequently with DMF, MeOH, DCM, Et₂O, and dried under reduced pressure to give 6-aminohexanoic acid loaded wang resin (Theoretical loading, 0.74 mmol/g).

To a suspension of the above 6-aminohexanoic acid loaded wang resin (2.55 g, 1.89 mmol) and (2S)-5-(benzyloxycarbony)amino-2-(9-fluorenyl-methoxycarbonylamino)pentanoic acid (2.77 g, 5.67 mmol) in NMP (25 mL), was added HATU (2.15 g, 5.67 mmol) and DIEA (2.02 mL, 11.34 mmol). The mixture was shaken f or 24 hours at room temperature. The solvent was drained, washed well subsequently with DMF, MeOH, DCM, Et₂O, and dried under reduced pressure. The resulted resin was treated with 20% piperidine in DMF (25 mL×2) for 1 hour to remove Fmoc group. Then, the solvent was drained, washed well subsequently with DMF, MeOH, DCM, Et₂O, and dried under reduced pressure to the target compound.

### EXAMPLE 249-2

### 6-[((2S)-5-{[(Benzyloxy)carbonyl]amino}-2-{[(2-naphthyloxy)carbonyl]amino}pentanoyl)amino]-hexanoic acid

To a suspension of 6-[((2S)-5-{[(benzyloxy)-carbonyl]amino}-2-amino}pentanoyl) amino]hexanoic acid loaded wang resin (1.89 mmol) obtained in Example 249-1 and pyridine (917.2 μL, 11.34 mmol) in DCM (25 mL), was added 2-naphthyl chloroformate (1.17 g, 5.67 mmol). The mixture was shaken for 2 days at room temperature.

The solvent was drained, washed well subsequently with DMF, MeOH, DCM, Et₂O, and dried under reduced pressure. After treated with 50% TFA in DCM (20 mL) for 1 hour, the resin was filtered and washed with DCM (15 mL×2). The filtrates were combined, evaporated and purified by HPLC (reverse phase C₁₈, 5 μ, 30 mm ×50 mm column, 254 nm, gradient 10-90% 0.05% TFA in CH₃CN/0.05% TFA in H₂O, 40 mL/min.). The fractions containing the target compound were combined, evaporated, and dried under reduced pressure to give the target compound.

MS ((+)ESI) m/z: 572 (M+Na)⁺.

¹H-NMR (DMSO-d₆): δ 1.20-1.70 (10H, m), 2.19 (2H, t, J=7.3 Hz), 2.95-3.15 (4H, m), 3.90-4.05 (1H, m), 5.02 (2H, s), 7.25-7.65 (10H, m), 7.85-8.05 (5H, m), 12.02 (1H, s).

### EXAMPLE 250

### 6-({(2S)-5-{[(Benzyloxy)carbonyl]amino}-2-[(4-biphenylylsulfonyl)amino]pentanoyl}amino)hexanoic acid

To a suspension of 6-[((2S)-5-{[(benzyloxy)carbonyl]amino}-2-amino}pentanoyl)amino]hexanoic acid loaded wang resin (1.89 mmol) obtained in Example 249-1 and pyridine (917.2 μL, 11.34 mmol) in DCM (25 mL), was added 4-biphenylsulfonyl chloride (1.43 g, 5.67 mmol). The mixture was shaken for 2 days at room temperature.

The solvent was drained, washed well subsequently with DMF, MeOH, DCM, Et₂O, and dried under reduced pressure. After treated with 50% TFA in DCM (20 mL) for 1 hour, the resin was filterd and washed with DCM (15 mL×2). The filtrates were combined, evaporated, and purified by HPLC (reverse phase C₁₈, 5 μ, 30 mm×50 mm column, 254 nm, gradient 10-90% 0.05% TFA in CH₃CN/0.05% TFA in H₂O, 40 mL/min.). The fractions containing the target compound were combined, evaporated and dried under reduced pressure to give the target compound.

MS ((−)ESI) m/z: 594 (M−H)⁻.

¹H-NMR (DMSO-d₆): δ 1.10-1.50 (10H, m), 2.09 (2H, t, J=7.3 Hz), 2.70-2.85 (2H, m), 2.85-3.00 (2H, m), 3.55-3.75 (1H, m), 4.98 (2H, s), 7.20-7.55 (9H, m), 7.65-8.00 (8H, m), 11.99 (1H, br-s).

### EXAMPLE 251

### 6-((2S)-5-{(Benzyloxy)carbonyl]amino}-2-{(4′-hydroxy-4-biphenylyl) carbonyl]amino}pentanoyl)-amino]hexanoic acid

To a suspension of 6-[((2S)-5-{[(benzyloxy)-carbonyl]amino}-2-amino}pentanoyl)amino]hexanoic acid loaded wang resin (1.89 mmol) obtained in Example 249-1, 4-(4-hydroxyphenyl)benzoic acid (1.21 g, 5.67 mmol) and HATU (2.15 g, 5.67 mmol) in NMP (20 mL), was added DIEA (2-02 mL, 11.34 mmol). The mixture was shaken for 2 days at room temperature.

The solvent was drained, washed well subsequently with DMF, MeOH, DCM, Et₂O, and dried under reduced pressure. After treated with 50% TFA in DCM (20 mL) for 1 hour, the resin was filterd and washed with DCM (15 mL×2). The filtrates were combined, evaporated and purified by HPLC (reverse phase C₁₈, 5 μ, 30 mm×50 mm column, 254 nm, gradient 10-90% 0.05% TFA in CH₃CN/0.05% TFA in H₂O, 40 mL/min.). The fractions containing the target compound were combined, evaporated, and dried under reduced pressure to give the target compound.

MS ((−)ESI) m/z: 574 (M−H)⁻.

¹H-NMR (DMSO-d₆): δ 1.20-1.80 (10H, m), 2;18 (2H, t, J=7.3 Hz), 2.95-3-15 (4H, m), 4.30-4.45 (1H, m), 5.00 (2H, s), 6.87 (2H, d, J=8.6 Hz), 7.20-7.35 (6H, m), 7.57 (2H, d, J=8.6 Hz), 7.67 (2H, d, J=8.3 Hz), 7.94 (2H, d, J=8.3 Hz), 8.37 (1H, d, J=8.0 Hz),9.66 (1H, s), 12.00 (1H, br-s).

### EXAMPLE 252-1

### 3-{2-[((2S)-2-Amino]-5-{[(4-methylphenyl)diphenyl-methyl]amino}pentanoyl)amino]phenyl}propanoic acid loaded resin

To a suspension of 4-(4-formyl-3-methoxyphenoxy)-butylyl AM resin (18 g, 0.51 mmole/g) in a mixture of THF (200 mL) and MeOH (5 mL), was added NaBH₄ (695 mg, 18.37 mmol). The mixture was shaken for 24 hours at room temperature. The resin was collected by filtration, washed well subsequently with DMF, MeOH, DCM, Et₂O, and dried under reduced pressure.

To the suspension of the above resin, 2-nitrocynnamic acid (2.66 g, 13.77 mmol) and triphenylphpsphine (3.61 g, 13.77 mmol) in THF (200 mL) was added DEAD (2.17 mL, 13.77 mmol). The mixture was shaken for 24 hours at room temperature. After drained the solvent, the resin was washed well with THF, and the carboxylic acid loading reaction was repeated. The resin was collected by filtration, washed well subsequently with DMF, MeOH, DCM, Et₂O, and dried under reduced pressure.

After treatment with a mixture of Ac₂O (17.36 mL, 18.36 mmol) and pyridine (29.7 mL, 36.72 mmol) in DCM (20 mL) for 24 hours at room temperature, to the resulted resin was added 2M SnCl₂—H₂O in DMF (150 mL×2) for 2 hours. Then, the resin was collected by filtration, washed well subsequently with DMF, MeOH, DCM, Et₂O, and dried under reduced pressure to give 2-aminocinnamic acid loaded resin.

To a suspension of the above 2-aminocinnamic acid loaded resin (9.18 mmol) and (2S)-2-(9-fluorenylmethoxycarbonyl)amino-5-{(4-methylphenyl)diphenylmethyl]amino}pentanoic acid (16.8 g, 27.54 mmol) and PyBroP (12.84 g, 27.54 mmol) in DMF (20 mL), was added DIEA (9.83 mL, 55.08 mmol). The mixture was shaken for 2 days at room temperature. The resin was collected by filtration, washed well subsequently with DMF, MeOH, DCM, Et₂O, and dried under reduced pressure. After the removal of Fmoc group with 20% piperidine in DMF (150 mL×2) for 1 hour, the resin was collected by filtration, washed well subsequently with DMF, MeOH, DCM, Et₂O, and dried under reduced pressure to give the target compound.

### EXAMPLE 252-2

### 3-{2-[((2S)-2-[(1-Benzofuran-2-ylcarbonyl)amino]-5-{[(benzyloxy)carbonyl]amino}pentanoyl)amino]-phenyl}propanoic acid

To a suspension of 3-{2-[((2S)-2-amino]-5-{[(4-methylphenyl)diphenylmethyl]amino}pentanoyl)-amino]phenyl}propanoic acid loaded resin (4.59 mmol) obtained in Example 252-1, 1-benzofuran-2-carboxylic acid (2.24 g, 13.77 mmol) and HATU (5.24 g, 13.77 mmol) in NMP (100 mL), was added DIEA (4.92 mL, 27.54 mmol). The mixture was shaken for 4 days at room temperature. The resin was collected by filtration, washed well subsequently with DMF, MeOH, DCM, Et₂O, and dried under reduced pressure. After treated with 5% TFA in DCM (100 mL) for 1 hour, the resin was filterd and washed with DCM (50 mL×2). The filtrates were combined, evaporated and purified by HPLC (reverse phase C₁₈, 5 μ, 30 mm×50 mm column, 254 nm, gradient 10-90% 0.1% TFA in CH₃CN/0.1% TFA in H₂O, 40 mL/min.). The fractions containing the target compound were combined, evaporated, and dried under reduced pressure to give 3-{2-[((2S)-2-[(1-Benzofuran-2-ylcarbonyl)amino]-5-aminopentanoyl)amino]phenyl}propanoic acid (200 mg).

A mixture of the above 3-{2-[((2S)-2-[(1-Benzofuran-2-ylcarbonyl)amino]-5-aminopentanoyl)amino]phenyl}propanoicacid (190 mg, 0.45 mmol) and 10% palladium on carbon (50% wet, 20 mg) in MeOH (5 mL) was hydrogenated at atmospheric pressure of hydrogen at room temperature After 4 hours, the catalyst was removed by filtration and evaporated to give residue, which was dissolved in DCM (30 mL). To the resulting mixture was added 1-(benzyloxycarbonyloxy)benzotriazole-6-carboxamid omethyl polystyrene (2.42 g, 0.93 mmole/g) and shaken for 1 week at room temperature. The resin was removed by filtration and evaporation of the solvent gave a residue, which was purified by HPLC (reverse phase C₁₈, 5 μ, 30 mm×50 mm column, 254 nm, gradient 10-90% 0.1% TFA in CH₃CN/0.1% TFA in H₂O, 40 mL/min.). The fractions containing the target compound were combined, evaporated, and dried under reduced pressure to give the target compound (63.2 mg)

MS ((−)ESI) m/z: 556 (M−H)⁻.

¹H-NMR (DMSO-d₆): δ 1.45-2.05 (4H, m), 2.40-2.55 (2H, m), 2.81 (2H, t, J=7.5 Hz), 3.00-3.15 (2H, m), 4.60-4.75 (1H, m), 5.00 (2H, s), 7.15-7.55 (12H, m), 7.65-7.85 (3H, m), 8.7 5 (1H, d, J=7.7 Hz), 9.60 (1H, S), 12.15 (1H, br-s).

### EXAMPLE 253

### 3-{2-((2S)-5-{(Benzyloxy)carbonyl]amino}-2-{[(4-biphenylylamino)carbonyl]amino}pentanoyl) amino]-phenyl}propanoic acid

A suspension of 3-{2-[((2S)-2-amino]-5-{[(4-methylphenyl)diphenylmethyl]amino}pentanoyl)-amino]phenyl}propanoic acid loaded resin (4.59 mmol) obtained in Example 252-1 and 4-biphenylyl isocyanate (2.69 g, 13.77 mmol) in DCM (100 mL) was shaken for 4 days at room temperature. The resin was collected by filtration, washed well subsequently with DMF, MeOH, DCM, Et₂O, and dried under reduced pressure. After treated with 5% TFA in DCM (100 mL) for 1 hour, the resin was filtered, and washed with DCM (50 mL×2) The filtrates were combined, evaporated, and purified by HPLC (reverse phase C₁₈, 5 μ, 30 mm×50 mm column, 254 nm, gradient 10-90% 0.1% TFA in CH₃CN/0.1% TFA in H₂O, 40 mL/min.). The fractions containing the desired compound were combined, evaporated, and dried under reduced pressure to give 3-{2-[((2S)-5-amino-2-{[(4-biphenylylamino)carbonyl]amino}pentanoyl)-amino]phenyl}acrylic acid (105 mg).

A mixture of the above 3-12-[((2S)-5-amino-2-{[(4-biphenylylamino)carbonyl]amino}pentanoyl)-amino]phenyl}acrylic acid (95 mg, 0.20 mmol) and 10% palladium on carbon (50% wet, 10 mg) in MeOH (5 mL) was hydrogenated at atmospheric pressure of hydrogen at room temperature. After 4 hours, the catalyst was removed by filtration and evaporated to give residue, which was dissolved in DCM (20 mL). To the resulting mixture was added 1-(benzyloxycarbonyloxy)-benzotriazole-6-carboxamidomethyl polystyrene (1.08 g, 0.93 mmole/g), and the mixture was shaken for 1 week at room temperature. The resin was removed by filtration and evaporation of the solvent gave a residue, which was purified by HPLC (reverse phase C₁₈ , 5 μ, 30 mm×50 mm column, 254 nm, gradient 10-90% 0.1% TFA in CH₃CN/0.1% TFA in H₂O, 40 mL/min.). The fractions containing the target compound were combined, evaporated, and dried under reduced pressure to give the target compound (12.4 mg).

MS ((−) ESI) m/z: 607 (M−H)⁻.

¹H-NMR (DMSO-d₆): δ 1.45-2.05 (4H, m), 2.40-2.55 (2H, m), 2.81 (2H, t, J=7.5 Hz), 3.00-3.15 (2H, m), 4.40-4.60 (1H, m), 5.00 (2H, s), 6.55 (1H, d, J=7.6 Hz), 7.10-7.65 (19H, m), 8.81 (1H, s), 9.63 (1H, s), 12.17 (1H, br-s).

In order to illustrate the usefulness of the object Compound (I), the pharmacological test is carried out as shown in the following.

### TESTS EXAMPLE

Binding assay using membrane preparation with the 10 expression of prostanoid receptor subtype

[I] Test Compound:

Sodium 6-{(2S)-2-[(1-benzofuran-2-yl-carbonyl)-amino]-5-[benzyloxycarbonylamino]pentanoylamino}-hexanoate (Example 23)

[II] Test Method:

The membrane fraction was prepared using COS-7 cells transfected prostanoid receptor subtype (human EP4)

The standard assay mixture contained membrane fraction, [³H]-PGE₂ in final volume of 0.25 mL was incubated for 1 hour at 3° C. The reaction was terminated by that the mixture was rapidly filtered through a glass filter (GF/B). Then the filter was washed with 4 mL of ice-cooled buffer two times. The radioactivity associated with the filter was measured by liquid scintillation counting.

In the experiment for competition of specific [³H]-PGE₂ was added at a concentration of 10 nM. The following buffer was used in all reactions.

Buffer: 20 mM Mes (pH 6.0), 1 mM EDTA, 10 mM MgCl₂

The inhibition (%) of the compound at a concentration of 10 nM was shown below.

[III] Test Result:

The test compound (1.0×10⁻⁸ M) showed the inhibition of 80% or more.

It appeared, from the above-mentioned inhibition test, that Compound (I) or pharmaceutically acceptable salt thereof of the present invention binds to PGE₂ receptor subtype, especially EP4, preferentially more than PGE₂. Therefore, Compound (I) of the present invention has an activating or inhibiting activity of PGE₂ receptor subtype.

In consequence, Compound (I) or pharmaceutically acceptable salt thereof is useful for treating or preventing diseases mediated by PGE₂2 more particularly useful for treating or preventing kidney dysfunction (e.g., acute nephritic syndrome, recurrent or persistent hematuria, chronic nephritic syndrome, nephritic syndrome, rapidly progressive nephritic syndrome, acute renal failure, chronic renal failure), inflammation and pain in joint and muscle (e.g., rheumatoid arthritis, rheumatoid spondylitis, osteoarthritis, gouty arthritis, juvenile arthritis), inflammatory skin condition (e.g., sunburn, burns, eczema, dermatitis), inflammatory eye condition (e.g., conjunctivitis), lung disorder in which inflammation is involved (e.g., asthma, bronchitis, pigeon fancier's disease, farmer's lung), condition of the gastrointestinal tract associated with inflammation (e.g., aphthous ulcer, Chrohn's disease, atrophic gastritis, gastritis varialoforme, ulcerative colitis, coeliac disease, regional ileitis, irritable bowel syndrome), gingivitis, inflammation, nephrithis, pain and tumescence after operation or injury, pyrexia, pain and other conditions associated with inflammation, allergic disease, systemic lupus erythematosus, scleroderma, polymyositis, tendinitis, bursitis, periarteritis nodose, rheumatic fever, Sjgren's syndrome, Behcet disease, thyroiditis, type I diabetes, diabetic complication (e.g., diabetic microangiopathy, diabetic retinopathy, diabetic nephropathy), nephrotic syndrome, aplastic anemia, myasthenia gravis, uveitis, contact dermatitis, psoriasis, Kawasaki disease, sarcoidosis, Hodgkin's disease, Alzheimers disease, migraine, liver dysfunction (e.g., hepatitis, cirrhosis), gastrointestinal dysfunction (e.g., diarrhea, inflammatory bowel diseases), shock, bone disease characterized by abnormal bone metabolism such as osteoporosis (especially, postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia cancer, cancer cachexia, breast cancer, calculosis, lithiasis (especially, urolithiasis), solid caricinoma, neurodegenerative disorder, sleeping disorder, hyperaldosteronism sexual dysfunction, or the like in human being or animal.

The Compound (I) of the present invention or its salts is also useful for the preparation of medicament having diuretic activity, which are useful for the preparation of drugs indicated treating or preventing various edema (e.g. cardiac edema, cerebral edema), hypertension such as malignant hypertension or the like, premenstrual tension, urinary calculus, oliguria such as the one caused by acute or chronic failure, hyperphosphaturia, or the like.

1. A compound of the formula (I): 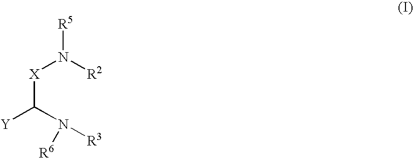
wherein
X is —CO— or —(CH₂)ₖ— (wherein k is 1, 2 or 3);
Y is
(1) lower alkyl, or
(2) Z-(CH₂)ₙ—,
{wherein
Z is
(1) aryl, or
(2) R¹—CO—NR⁴—
(wherein
R¹ is (1) aryl, heterocyclyl, aryl-(lower alkyl), aryl-(lower alkoxy), or heterocyclyl-(lower alkoxy), each of which may be substituted with one or more substituent(s) selected from the group consisting of
(a) lower alkyl,
(b) halogen and
(c) hydroxy; or
(2) lower alkoxy; and
R⁴ is hydrogen, or lower alkyl); and
n is 1, 2, 3, 4, 5 or 6};
R² is (1) lower alkyl, aryl-(lower alkyl) or (lower alkyl)thio-(lower alkyl), each of which may be substituted with one or more substituent(s) selected from the group consisting of
(a) heterocyclyl,
(b) carboxy,
(c) carboxy-(lower alkyl),
(d) amidated carboxy,
(e) (lower alkoxy)carbonyl which may be substituted with cycloalkyl, heterocyclyl or (lower alkanoyl)oxy; and
(f) cyano; or
(2) aryl which may be substituted with lower alkyl, lower alkenyl, aryl, lower alkoxy, (lower alkyl)amino, (lower alkyl)thio, carboxy, (lower alkoxy)carbonyl, (lower alkoxy)-(lower alkyl), (lower alkyl)amino-(lower alkyl), or (lower alkyl)thio-(lower alkyl), each of which may be further substituted with one or more substituent(s) selected from the group consisting of
(a) heterocyclyl,
(b) (lower alkoxy)carbonyl,
(c) carboxy and (d) amidated carboxy;
R¹ is (1)-Q-R⁷,
[wherein
Q is —CO— or —SO₂—,
R⁷ is (a) lower alkyl which may be substituted with one or more substituent(s) selected from the group consisting of cycloalkyl, aryl which may be further substituted with aryl(s), and heterocyclyl,
(b) lower alkenyl which may be substituted with one or more substituent(s) selected from the group consisting of aryl and heterocyclyl,
(c) cycloalkyl,
(d) aryl which may be substituted with one or more substituent(s) selected from the group consisting of lower alkyl, aryl which may be further substituted with hydroxy(s), lower alkoxy, aryloxy, hydroxy, and halogen,
(e) heterocyclyl which may be substituted with one or more substituent(s) selected from the group consisting of lower alkyl, aryl which may be further substituted with halogen(s), and halogen,
(f) aryloxy, or
(g) amino which may be substituted with aryl(s) which may be further substituted with one or more substituent(s) selected from the group consisting of aryl and heterocyclyl]; or
(2) lower alkyl which may be substituted with aryl(s) or heterocyclyl(s), each of which may be further substituted with aryl(s); and
R⁵ and R⁶ are independently hydrogen or lower alkyl; or
R⁶ and Y may be linked together to form —(CH₂)ₘ—(wherein m is 2, 3, 4 or 5); or a pharmaceutically acceptable salt thereof.

2. A compound of claim 1 having the formula (Ia): 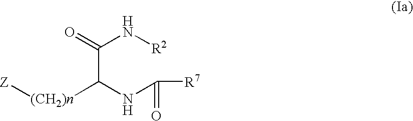
wherein Z, R², R⁷ and n are as defined above.

3. A compound of claim 1 having the formula (Ib): 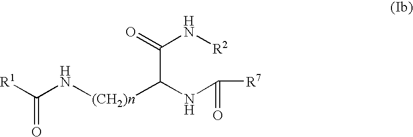
wherein R¹, R², R⁷ and n are as defined above.

4. A compound of claim 3,
wherein
R¹ is aryl-(lower alkoxy);
R² is lower alky, or
aryl which may be substituted with carboxy-(lower alkyl);
R⁷ is heterocyclyl which may be substituted with substituted with lower alkyl; and
n is 1, 2, 3, 4 or 5.

5. A compound selected from:
sodium 6-{(2S)-2-[(1-benzofuran-2-yl-carbonyl)amino]-5-[benzyloxycarbonylamino]pentanoylamino}-hexanoate,
(2E)-3-{2-[(2S)-2-[(1H-indol-2-ylcarbonyl)amino]-5-[benzyloxycarbonylamino]pentanoylamino]phenyl}-acrylic acid,
(2E)-3-{2-[(2S)-2-[(1-methyl-1H-indol-2-yl-carbonyl)amino]-5-[benzyloxycarbonylamino]-pentanoylamino]phenyl}acrylic acid,
3-{2-[(2S)-2-[(1-methyl-1H-indol-2-ylcarbonyl)-amino]-5-[benzyloxycarbonylamino]pentanoylamino]- phenyl}propanoic acid,
sodium 3-{2-[(2S)-2-[(2-quinolinylcarbonyl)amino]-5-[benzyloxycarbonylamino]pentanoylamino]phenyl}-propanoate,
6-[((2S)-2-[(1-benzofuran-2-ylcarbonyl)amino]-5-{[(benzyloxy)carbonyl]amino}pentanoyl)amino]-2-naphthoic acid,
3-{2-[((2S)-5-{[(benzyloxy)carbonyl]amino}-2-{[(8-methylimidazo[1,2-a]pyridin-2-yl)carbonyl]amino}-pentanoyl)amino]phenyl}propanoic acid,
3-[2-({(2S)-5-{[(benzyloxy)carbonyl]amino}-2-[(2-quinolinylmethyl)amino]pentanoyl}amino)-phenyl]propanoic acid, and
3-[2-({(2S)-5-{[(benzyloxy)carbonyl]amino}-2-[(1H-indol-2-ylcarbonyl)amino]pentanoyl}amino)phenyl]-propanoic acid.

6. A process for preparing the compound of the formula (Ia-1): 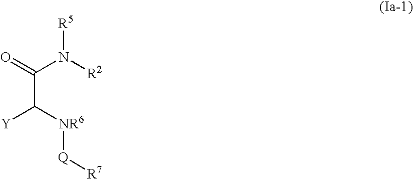
wherein
Y is
(1) lower alkyl, or
(2) Z-(CH₂)ₙ—,
{wherein
Z is
(1) aryl, or
(2) R¹—CO—NR⁴—
(wherein
R¹ is (1) aryl, heterocyclyl, aryl-(lower alkyl), aryl-(lower alkoxy), or heterocyclyl-(lower alkoxy), each of which may be substituted with one or more substituent(s) selected from the group consisting of
(a) lower alkyl,
(b) halogen and
(c) hydroxy; or
(2) lower alkoxy; and
R⁴ is hydrogen, or lower alkyl); and
n is 1, 2, 3, 4, 5 or 6};
Q is —CO— or —SO₂—;
R² is (1) lower alkyl, aryl-(lower alkyl) or (lower alkyl)thio-(lower alkyl), each of which may be substituted with one or more substituent(s) selected from the group consisting of
(a) heterocyclyl,
(b) carboxy,
(c) carboxy-(lower alkyl),
(d) amidated carboxy,
(e) (lower alkoxy)carbonyl which may be substituted with cycloalkyl, heterocyclyl or (lower alkanoyl)oxy; and
(f) cyano; or
(2) aryl which may be substituted with lower alkyl, lower alkenyl, aryl, lower alkoxy, (lower alkyl)amino, (lower alkyl)thio, carboxy, (lower alkoxy)carbonyl, (lower alkoxy)-(lower alkyl), (lower alkyl)amino-(lower alkyl), or (lower alkyl)thio-(lower alkyl), each of which may be further substituted with one or more substituent(s) selected from the group consisting of
(a) heterocyclyl,
(b) (lower alkoxy)carbonyl,
(c) carboxy and
(d) amidated carboxy;
R⁵ and R⁶ are independently hydrogen or lower alkyl; or
R⁶ and Y may be linked together to form —(CH₂)ₘ—(wherein m is 2, 3, 4 or 5); and
R⁷ is (a) lower alkyl which may be substituted with one or more substituent(s) selected from the group consisting of
cycloalkyl,
aryl which may be further substituted with aryl(s), and
heterocyclyl,
(b) lower alkenyl which may be substituted with one or more substituent(s) selected from the group consisting of aryl and heterocyclyl,
(c) cycloalkyl,
(d) aryl which may be substituted with one or more substituent(s) selected from the group consisting of
lower alkyl,
aryl which may be further substituted with hydroxy(s),
lower alkoxy,
aryloxy,
hydroxy, and
halogen,
(e) heterocyclyl which may be substituted with one or more substituent(s) selected from the group consisting of
lower alkyl,
aryl which may be further substituted with halogen(s), and
halogen,
(f) aryloxy, or
(g) amino which may be substituted with aryl(s) which may be substituted with one or more substituent(s) selected from the group consisting of aryl and heterocyclyl];
or a pharmaceutically acceptable salt thereof,
comprising, reacting a compound (IIa): 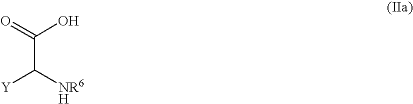
(wherein Y and R⁶ are each as defined above), or its reactive derivative at the carboxy group or the salt thereof, with a compound (IIIa): 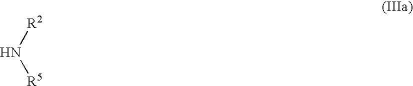
(wherein R² and R⁵ are each as defined above), or its reactive derivative at the amino group or the salt thereof to give a compound (IVa): 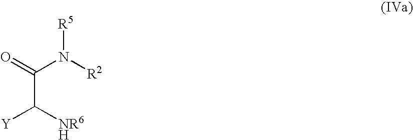
(wherein Y, R², R⁵ and R⁶ are each as defined above), or its salt; and
reacting the compound (IVa): 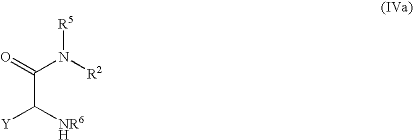
(wherein Y, R², R⁵ and R⁶ are each as defined above), or its salt, with a compound (V): 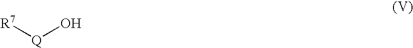
(wherein Q and R⁷ are each as defined above), or its reactive derivative at the carboxy group (in case of Q is —CO—)/the sulfo group (in case of Q is —SO₂—), or the salt thereof.

7. A process for preparing the compound of the formula (Ib-1): 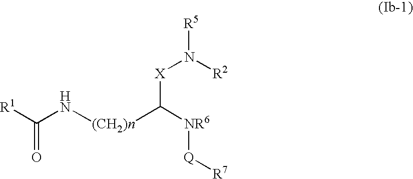
wherein
X is —CO—, or —(CH₂)ₖ— (wherein k is 1, 2 or 3);
Q is —CO— or —SO₂—;
R¹ is (1) aryl, heterocyclyl, aryl-(lower alkyl), aryl-(lower alkoxy), or heterocyclyl-(lower alkoxy), each of which may be substituted with one or more substituent(s) selected from the group consisting of
(a) lower alkyl,
(b) halogen and
(c) hydroxy; or
(2) lower alkoxy; and
R² is (1) lower alkyl, aryl-(lower alkyl) or (lower alkyl)thio-(lower alkyl), each of which may be substituted with one or more substituent(s) selected from the group consisting of
(a) heterocyclyl,
(b) carboxy,
(c) carboxy-(lower alkyl),
(d) amidated carboxy,
(e) (lower alkoxy)carbonyl which may be substituted with cycloalkyl, heterocyclyl or (lower alkanoyl)oxy; and
(f) cyano; or
(2) aryl which may be substituted with lower alkyl, lower alkenyl, aryl, lower alkoxy, (lower alkyl)amino, (lower alkyl)thio, carboxy, (lower alkoxy)carbonyl, (lower alkoxy)-(lower alkyl), (lower alkyl)amino-(lower alkyl), or (lower alkyl)thio-(lower alkyl), each of which may be further substituted with one or more substituent(s) selected from the group consisting of
(a) heterocyclyl,
(b) (lower alkoxy)carbonyl,
(c) carboxy and
(d) amidated carboxy;
R⁵ and R⁶ are independently hydrogen or lower alkyl; or
R⁶ and Y may be linked together to form —(CH₂)ₘ— (wherein m is 2, 3, 4 or 5);
R⁷ is (a) lower alkyl which may be substituted with one or more substituent(s) selected from the group consisting of
cycloalkyl,
aryl which may be further substituted with aryl(s), and
heterocyclyl,
(b) lower alkenyl which may be substituted with one or more substituent(s) selected from the group consisting of aryl and heterocyclyl,
(c) cycloalkyl,
(d) aryl which may be substituted with one or more substituent(s) selected from the group consisting of
lower alkyl,
aryl which may be further substituted with hydroxy(s),
lower alkoxy,
aryloxy,
hydroxy, and
halogen,
(e) heterocyclyl which may be substituted with one or more substituent(s) selected from the group consisting of
lower alkyl,
aryl which may be further substituted with halogen(s), and
halogen,
(f) aryloxy, or
(g) amino which may be substituted with aryl(s) which may be substituted with one or more substituent(s) selected from the group consisting of aryl and heterocyclyl]; and
n is 1, 2, 3, 4, 5 or 6;
or a pharmaceutically acceptable salt thereof, comprising, reacting a compound (IIb): 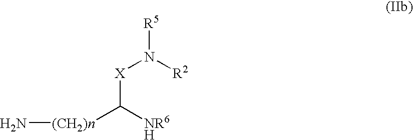
(wherein X, R², R⁵, R⁶ and n are each as defined above), or its reactive derivative at the amino group or the salt thereof, with a compound (IIIb): 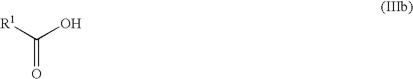
(wherein R¹ is as defined above), or its reactive derivative at the carboxy group or the salt thereof to give a compound (IVb): 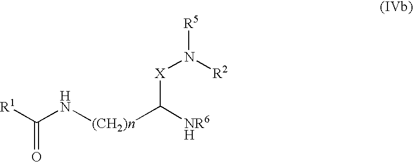
(wherein X, R¹, R², R⁵, R⁶, n and are as defined above), or its salt; and reacting the compound (IVb): 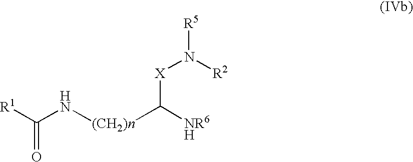
(wherein X, R¹, R², R⁵, R⁶ and n are as defined above), or its salt, with a compound (V): 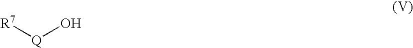
(wherein Q and R⁷ are as defined above), or its reactive derivative at the carboxy group (in case of Q is —CO—)/the sulfo group (in case of Q is —SO₂—), or the salt thereof.

8. A process for preparing the compound of the formula (Ia-2): 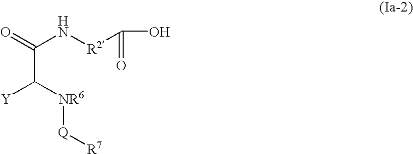
wherein
Y is
(1) lower alkyl, or
(2) Z-(CH₂)ₙ—,
{wherein
Z is
(1) aryl, or
(2) R¹—CO—NR⁴—
(wherein
R¹ is (1) aryl, heterocyclyl, aryl-(lower alkyl), aryl-(lower alkoxy), or heterocyclyl-(lower alkoxy), each of which may be substituted with one or more substituent(s) selected from the group consisting of
(a) lower alkyl,
(b) halogen and
(c) hydroxy; or
(2) lower alkoxy; and
R⁴ is hydrogen, or lower alkyl); and
n is 1, 2, 3, 4, 5 or 6};
Q is —CO— or —SO₂—;
R^{2′} is (1) lower alkyl, (lower alkyl)thio-(lower alkyl) or aryl-(lower alkyl); or
(2) aryl which may be substituted with lower alkyl, lower alkenyl, aryl, lower alkoxy, (lower alkyl)amino, (lower alkyl)thio, (lower alkoxy)-(lower alkyl), (lower alkyl)amino-(lower alkyl), or [(lower alkyl)thio]-(lower alkyl);
R⁶ is hydrogen or lower alkyl; or
R⁶ and Y may be linked together to form —(CH₂)ₘ—(m is 2, 3, 4 or 5);
R⁷ is (a) lower alkyl which may be substituted with one or more substituent(s) selected from the group consisting of
cycloalkyl,
aryl which may be further substituted with aryl(s), and heterocyclyl,
(b) lower alkenyl which may be substituted with one or more substituent(s) selected from the group consisting of aryl and heterocyclyl,
(c) cycloalkyl,
(d) aryl which may be substituted with one or more substituent(s) selected from the group consisting of
lower alkyl,
aryl which may be further substituted with hydroxy(s),
lower alkoxy,
aryloxy,
hydroxy, and
halogen,
(e) heterocyclyl which may be substituted with one or more substituent(s) selected from the group consisting of
lower alkyl,
aryl which may be further substituted with halogen(s), and
halogen,
(f) aryloxy, or
(g) amino which may be substituted with aryl(s) which may be substituted with one or more substituent(s) selected from the group consisting of aryl and heterocyclyl];
or a pharmaceutically acceptable salt thereof,
comprising, reacting a compound (IIa): 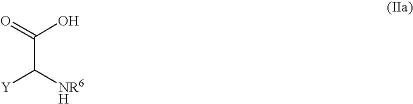
(wherein Y and R⁶ are each as defined above), or its reactive derivative at the carboxy group or the salt thereof, with a resin-bound compound (IIIc): 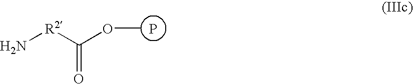
(wherein R^{2′} is as defined above, and \textcircled{P} is polymer), or its reactive derivative at the amino group or the salt thereof to give a compound (IVc): 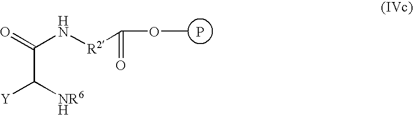
(wherein Y, \textcircled{P} R^{2′} and R⁶ are as defined above), or its salt;
reacting the compound (IVc): 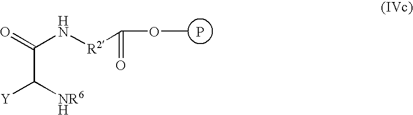
(wherein Y, \textcircled{P} R^{2′} and R⁶ are as defined above), or its salt, with a compound (V): 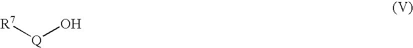
(wherein Q and R⁷ are as defined above), or its reactive derivative at the carboxy group (in case of Q is —CO—)/the sulfo group (in case of Q is —SO₂—), or the salt thereof to give a compound (Ia-2′): 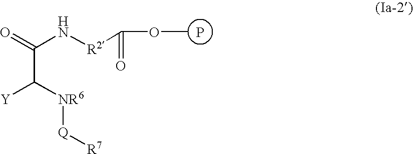
(wherein Q, Y, \textcircled{P}, R^{2′}, R⁶, and R⁷ are as defined above), or its salt; and
subjecting the compound (Ia-2′): 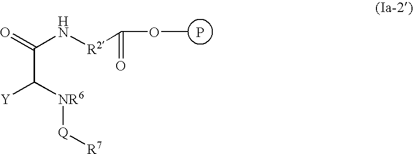
(wherein Q, Y, \textcircled{P}, R^{2′}, R⁶, and R⁷ are as defined above), or its salt to a cleavage reaction of the resin.

9. A compound of claim 1 for use as a medicament.

10. The compound of claim 9 for use in the treatment and/or prevention of PGE₂ mediated diseases in human beings or animals.

11. A medicament comprising a compound of claim 1 as an active ingredient.

12. A pharmaceutical composition comprising a compound of claim 1 as an active ingredient, in association with a pharmaceutically acceptable carrier or excipient.

13. An agonist or antagonist of PGE2 consisting of a compound of claim 1.

14. A method for treatment and/or prevention of PGE₂ mediated diseases which comprises administering an effective amount of the compound of claim 1 to human beings or animals.

15. A method for treating or preventing kidney dysfunction, inflammatory conditions, various pains, collagen diseases, autoimmune diseases, various immunity diseases, analgesic, thrombosis, allergic disease, cancer or neurodegenerative diseases which comprises administering an effective amount of a compound of claim 1 to human beings or animals.

16. The method of using a compound of claim 1 as a medicament.

17. The method of using a compound of claim 1 as an agonist or an antagonist of PGE₂-sensitive receptor.

18. Use of The method of using the compound of claim 1 for treatment and/or prevention of PGE₂ mediated diseases in human beings or animals.

19. A commercial package comprising the pharmaceutical composition containing the compound identified in claim 1 and a written matter associated therewith, wherein the written matter states that the compound (I) can or should be used for preventing or treating PGE₂ mediated diseases.
